(19) United States
(12) Patent Application Publication
(10) Pub. No.: US 2025/0296901 A1
(43) Pub. Date: Sep. 25, 2025

(54) REACTOR SYSTEMS FOR OXIDATIVE DEHYDROGENATION (ODH) OF ETHANE

(71) Applicant: NOVA CHEMICALS (INTERNATIONAL) S.A., Fribourg (CH)

(72) Inventors: Mohammad KESHTKAR, SW Calgary (CA); Bolaji OLAYIWOLA, SW Calgary (CA); David GENT, Red Deer (CA); Vasily SIMANZHENKOV, NW Calgary (CA)

(73) Assignee: NOVA CHEMICALS (INTERNATIONAL) S.A., Fribourg (CH)

(21) Appl. No.: 18/862,788

(22) PCT Filed: Apr. 6, 2023
(86) PCT No.: PCT/IB2023/053540

§ 371 Date: Nov. 4, 2024

Related U.S. Application Data
(60) Provisional application No. 63/338,367, filed on May 4, 2022.

(51) Int. Cl. C07C 5/48; C07C 51/215

(52) U.S. Cl. CPC C07C 5/48; C07C 51/215

(57) ABSTRACT

An oxidative dehydrogenation (ODH) reactor system and a method of operating the ODH reactor system, including providing feed having ethane, oxygen, and diluent to give a reaction mixture flowing through the tube side of the ODH reactor, and converting ethane into ethylene with ODH catalyst on the tube side. Coolant is routed through the shell side of the ODH reactor to maintain the tube side at a first temperature in a first cooling section and at a second temperature in a second cooling section, wherein the first temperature is lower than the second temperature. The ODH reactor system may include more than one ODH reactor. For ODH reactor systems having more than one ODH reactor is series, oxygen gas may be injected between ODH reactors.

### TECHNICAL FIELD

This disclosure relates to oxidative dehydrogenation (ODH) reactor systems to improve performance of the ODH plant for ethylene production.

### BACKGROUND ART

Catalytic oxidative dehydrogenation of alkanes into corresponding alkenes is an alternative to steam cracking. In contrast to steam cracking, oxidative dehydrogenation (ODH) may operate at lower temperature and generally does not produce coke. For ethylene production, ODH can provide a greater yield for ethylene than does steam cracking. The ODH may be performed in a reactor vessel having catalyst for the conversion of an alkane to a corresponding alkene.

Acetic acid as a byproduct may be generated in the conversion of the lower alkanes (e.g., ethane) into the corresponding alkenes (e.g., ethylene). The product alkene and byproduct acetic acid may each be recovered from the ODH reactor effluent.

Carbon dioxide is the primary greenhouse gas emitted through human activities. Carbon dioxide (CO₂) may be generated in various industrial and chemical plant facilities. At such facilities, utilization of energy more efficiently may reduce CO₂ emissions at the facility and therefore decrease the CO₂ footprint of the facility.

### SUMMARY OF INVENTION

An oxidative dehydrogenation (ODH) reactor system and a method of operating the ODH reactor system. In operation, feed having ethane, oxygen, and diluent is provided to give a reaction mixture flowing through the tube side of the ODH reactor. Ethane is converted into ethylene via ODH catalyst on the tube side. Coolant is routed through the shell side of the ODH reactor to maintain the tube side at a first temperature in a first cooling section and at a second temperature in a second cooling section, wherein the first temperature is lower than the second temperature.

The ODH reactor system may include more than one ODH reactor. For ODH reactor systems having more than one ODH reactor in series, oxygen gas may be injected between ODH reactors. The oxygen gas may be injected into the product effluent of the upstream ODH reactor that is fed to the downstream ODH reactor. In implementations, the inter-stage product effluent may be cooled to accommodate the oxygen injection.

The details of one or more implementations are set forth in the accompanying drawings and the description below. Other features and advantages will be apparent from the description and drawings, and from the claims.

### BRIEF DESCRIPTION OF DRAWINGS

FIG. 1 is a diagram of an ODH plant having an ODH reactor system that dehydrogenates ethane into ethylene.

FIGS. 2 and 2A are diagrams of a representation of an example multi-tubular fixed bed reactor.

FIGS. 3, 4, 4A, 5, 6, 7, 8, 9, 10, 11, and 12 are diagrams of ODH reactor systems that may be the ODH reactor system of FIG. 1.

FIG. 13 is a ternary plot of an example of a flammability diagram for mixtures of ethane, oxygen, and steam at 300° C. and 500 kilopascals (kPa) absolute.

FIGS. 14 and 15 are each a block flow diagram of a respective method of operating an ODH reactor system having at least one ODH reactor.

### DESCRIPTION OF EMBODIMENTS

Embodiments of the present techniques relate to oxidative dehydrogenation (ODH) reactor systems that dehydrogenate an alkane (e.g., ethane) into the corresponding alkene (e.g., ethylene). Aspects are directed to configurations of ODH reactor systems that reduce energy consumption at the ODH plant (facility).

For example, some aspects relate to configurations that reduce the amount of dilution steam in the alkane feed, thereby lowering energy consumption by the ODH plant.

The ODH reactor may dehydrogenate ethane to ethylene via an ODH catalyst in presence of oxygen. Ethylene may be separated from the ODH reactor effluent to give the product ethylene.

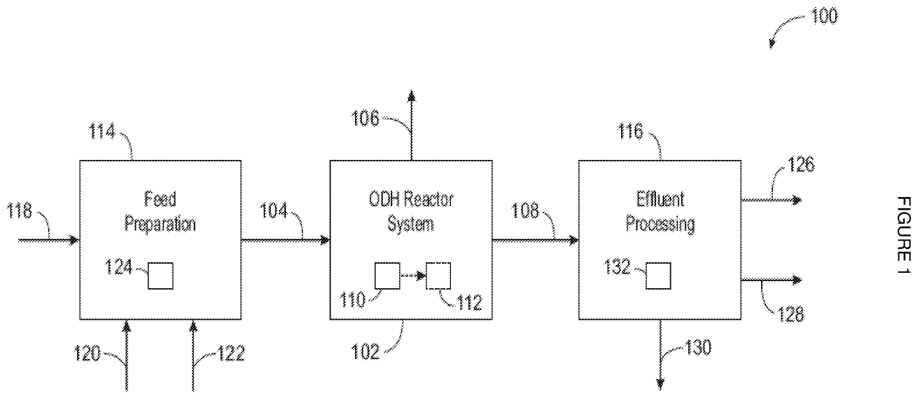

FIG. 1 is an ODH plant 100 having an ODH reactor system 102 that dehydrogenates ethane into ethylene. The dehydrogenation of ethane to ethylene may be a reaction of ethane with oxygen via an ODH catalyst to give the ethylene. The ODH reactor system 102 may be a single-reactor system in having a sole ODH reactor (e.g., Configurations 1-7 discussed below) or a multi-reactor system (e.g., Configurations 8-13 discussed below) in having more than one ODH reactor disposed operationally in series. An additional ODH reactor(s) may be included in parallel in Configurations 1-13, such to achieve a desired amount of ethylene production capacity at the ODH plant. The ODH reactor(s) may be a multi-tubular fixed bed reactor having the ODH catalyst on the tube side, and in which coolant (e.g., molten salt) flows on the shell side to remove the heat of reaction from the tube side.

In addition to configuring the ODH reactor system 102 in an effort to reduce the amount of dilution steam implemented in the feed 104 to reduce energy consumption of the ODH plant, considerations may also include increasing energy efficiency by recovering heat from the ODH reactor for generating steam 106 for the ODH plant 100. For instance, heat from the coolant discharged from the reactor (as heated in the shell side) and/or heat from the process effluent discharged from the ODH reactor (from the tube side) may be utilized to heat and vaporize boiler feedwater into steam.

The ODH reactor system 102 may have a sole ODH reactor 110 or can have additional ODH reactors 112 operationally in series. Moreover, additional similarly configured ODH reactors may be included operationally in parallel for increased ethylene production capacity. For a multi-reactor system, the first ODH reactor 110 in the series may discharge effluent as feed to a second ODH reactor 112. In certain implementations with the ODH reactor system 102 as a multi-reactor system, the effluent 108 may discharge from the final ODH reactor 112 (e.g., the second ODH reactor, the third ODH reactor, etc.) in the series. Diluent in the feed 104 reactor can include, for example, nitrogen gas (N₂), carbon dioxide gas (CO₂), or water (steam), or any combinations thereof. If the diluent in the feed 104 includes water, the water in the effluent 108 may include both unreacted diluent water and water generated in the ODH reaction in the ODH reactor(s).

In implementations, the feed 104 may include ethane and oxygen and is provided (e.g., conveyed in a conduit) to the ODH reactor 110. The feed 104 may include diluent (e.g., steam) to place the feed 104 outside of flammability limits. Steam as the diluent may be labeled as dilution steam. In preparation of the feed 104, energy (heating capacity) may be applied to heat or vaporize water to incorporate water vapor or steam as dilution steam into the feed 104. The feed 104 can be characterized as mixed feed including ethane, oxygen, and dilution steam.

The dilution steam that enters the ODH reactor system 102 in the feed 104 may discharge in the effluent 108 from the ODH reactor system 102. In the processing of the effluent 108 downstream of the reactor system 102, energy (cooling capacity) may be applied to condense the dilution steam in the effluent 108.

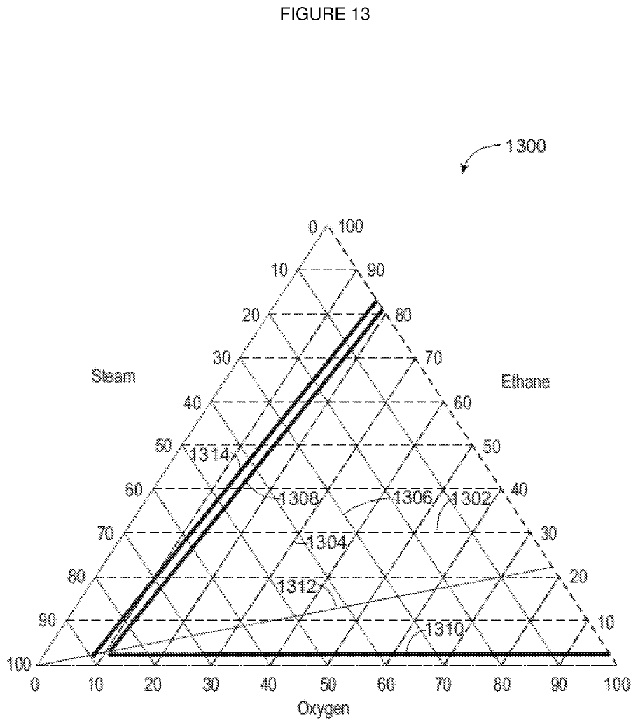

Significant demands of energy in the ODH plant 100 can be [1] heating water (e.g., in the feed preparation system 114) to provide the dilution steam in the feed 104, [2] condensing the dilution steam in the ODH reactor effluent 108 in the downstream effluent processing 116, and [3] separating condensed acetic acid from the condensed water. Therefore, reducing the amount of dilution steam in the feed 104 can decrease energy consumption at the ODH plant 100. Techniques to reduce the amount of dilution steam in the feed 104 include reducing the amount of oxygen gas in the feed 104. Advantageously, for feed 104 with less oxygen, less dilution steam places the feed 104 outside of flammability limits. See, e.g., FIG. 13.

Techniques reducing the amount of oxygen in the feed 104 can include increasing ethylene selectivity in favoring in the ODH reactor the formation of ethylene over formation of carbon monoxide (consumes more stoichiometric amount of oxygen than does ethylene formation) and over formation of carbon dioxide (consumes more stoichiometric amount of oxygen than does ethylene formation). Techniques favoring ethylene formation over formation of carbon monoxide and carbon dioxide can include operating one or more cooling sections of the ODH reactor at lower temperature where feasible and/or beneficial. This is so because higher temperature (of the reaction mixture and the ODH catalyst on the tube side) can more readily allow for the undesirable formation of carbon monoxide and carbon dioxide.

Techniques disfavoring formation of carbon monoxide and carbon dioxide can include increasing the heat transfer (and/or increasing efficiency of heat transfer, e.g., via an increased heat transfer coefficient) in the ODH reactor from the tube side to the shell side. This may avoid or reduce occurrence of temperature spikes on the tube side that can favor or cause (result in) the formation (unwanted) of carbon monoxide and carbon dioxide. To improve heat transfer can include limiting the temperature increase of coolant (e.g., molten salt) through the shell side of the ODH reactor so to have increased flow rate of the coolant through the shell side to increase the value of a heat transfer coefficient. To improve heat transfer can include limiting the diameter (e.g., nominal diameter, outside diameter, or inside diameter) of the tubes in the tube bundle in the ODH reactor to increase velocity of the reaction mixture flowing through the tubes. Such may give, for example, an increased Reynolds number (Re) that can increase the heat transfer coefficient. Again, an increased heat transfer coefficient may reduce occurrence of temperature spikes on the tube side. Temperature spikes on the tube side can undesirably result in more formation of carbon monoxide and carbon dioxide (and thus more consumption of oxygen).

The formation of carbon monoxide and carbon dioxide consume more stoichiometric amount of oxygen than does the formation of ethylene. Therefore, increased formation of carbon monoxide and carbon dioxide can lead to more oxygen in the feed 104, which undesirably leads to more dilution steam in the feed 104. More dilution steam in the feed 104 can mean more energy consumption at the ODH plant 100. The formation of carbon monoxide and carbon dioxide are unwanted reactions also because ethylene is a more valuable product. Carbon monoxide and carbon dioxide are generally undesirable products. The generation of carbon dioxide can increase the CO₂ footprint of the ODH plant (facility) 100.

The ODH plant 100 includes the feed preparation system(s) 114 that provide feed 104 to the sole or first ODH reactor 110 in the ODH reactor system 102. Again, the feed 104 may include ethane and oxygen. As mentioned, the feed 104 may include diluent that places the feed 104 outside of the flammability limits. See, for example, FIG. 13 that depicts a flammability diagram for mixtures of ethane, oxygen, and diluent as water (steam).

Examples of diluent that may be utilized to place the feed 104 stream outside of flammability limits may include water, nitrogen, carbon dioxide, helium, argon, methane, etc. In embodiments, water is the diluent. As indicated, the water as diluent may generally be in the form of steam. Steam or vaporized water can be an attractive diluent, for example, due to the relative simplicity of the separation of the water from the ODH reactor system 102 product stream (effluent) in implementations.

The feed preparation system 114 may incorporate the diluent with the ethane (and oxygen) to give the feed 104 conveyed to the ODH reactor system 102. For instances with the diluent as water, the feed preparation system 114 may heat or vaporize the water for incorporation into (addition to) the ethane (and oxygen) as dilution steam.

In implementations, ethane 118 (gas) and water 120 may be provided to the feed preparation system 114, and the water 120 incorporated as steam or water vapor into the ethane 118 to give the feed 104. Such water 120 as incorporated can be characterized or labeled as dilution steam. Oxygen 122 (gas) may be provided to the feed preparation system 114 for addition of the oxygen 122 to the ethane 118 to give the feed 104. In certain implementations, some or all of the oxygen 122 may be added to the conduit conveying the feed 104 to the ODH reactor system 102.

The feed preparation equipment 124 can include a heat exchanger to heat the water 118 as liquid (and vaporize the liquid water in some instances), an ethane saturator column to saturate ethane 118 with the water 120, a steam drum vessel to add steam (including water 120) to ethane 118, and so on. For feed dilution embodiments (e.g., in feed preparation 114) in heating the water 118, medium pressure (MP) steam may be employed which is a higher value (more expensive) steam than low pressure (LP) steam. Sources of LP steam and MP steam in the ODH plant 100 can include, for example, extraction turbines or a depressurizing valve for HP or VHP steam.

For addition of water vapor to the feed 104 to the ODH reactor, embodiments may employ, for example, a dilution steam drum or a saturator tower. A dilution steam drum may be more straightforward in providing dilution steam but can unfortunately rely on a higher value heat source such as medium pressure steam. In implementations, medium pressure steam can instead be better utilized, for example, to drive steam turbines. A saturator tower may saturate hydrocarbon (e.g., ethane) gas and/or oxygen gas with water vapor and utilize a relatively high circulation of water. For examples of feed dilution, see WO Published Patent Application No. WO 2022/229848 entitled “Integration for Feed Dilution in Oxidative Dehydrogenation (ODH) Reactor System”, which is incorporated by reference herein in its entirety.

Components in addition to ethylene that form in the ODH reactor may include acetic acid, carbon dioxide, carbon monoxide, and water. Thus, the ODH plant 100 may produce ethylene 126 and acetic acid 128. Carbon monoxide and carbon dioxide may undesirably form in the ODH reactor and discharge in the effluent 108 with the ethylene and acetic acid.

The ODH reactor (e.g., 110, 112) may generate ethylene (C₂H₄) as the main product and acetic acid (CH₃COOH) as a value-added coproduct. The ODH reactor (e.g., 110, 112) may generate carbon monoxide (CO), carbon dioxide (CO₂), and water (H₂O). The reaction mechanisms in the ODH reactor characterized as reactions of ethane (C₂H₆) and oxygen (O₂) can be explained as follows:

C₂H₆+0.5 O₂→C₂H₄+H₂O

C₂H₆+1.5 O₂→CH₃COOH+H₂O

C₂H₆+2.5 O₂→2 CO+3 H₂O

C₂H₆+3.5 O₂→2 CO₂+3 H₂O

As can be seen in the equations immediately above, the reactions forming CO and CO₂ generally utilize (consume) a greater stoichiometric amount of O₂ than does the listed reaction generating ethylene. Therefore, configuring the ODH reactor system 102 to increase ethylene selectivity in favoring ethylene formation over formation of CO and/or CO₂ may decrease the amount of O₂ implemented in the feed 104. A decrease in amount of O₂ in the feed 104 can mean less dilution steam implemented in the feed 104 to place the feed 104 outside of flammability limits (outside of the flammability envelope) (see, e.g., FIG. 13). As discussed, less dilution steam in the feed 104 can mean that energy consumption in the ODH plant is reduced.

The product effluent 108 from the ODH reactor system 102 may include ethylene, acetic acid, carbon dioxide, carbon monoxide, water, and unreacted ethane. In addition to dilution steam as water in the effluent 108, the effluent 108 may also include water (as water vapor or steam) formed in the ODH reactor.

The processing 116 of the effluent 108 may include condensing the water and acetic acid to remove the acetic acid and water from the effluent 108. The water and acetic acid may be condensed, for example, via a heat exchanger utilizing a cooling medium (e.g., cooling water, air, etc.). In some implementations, a flash drum vessel or quench tower vessel may facilitate separation of the liquid as condensed from the effluent 108.

The condensed water and the condensed acetic acid as a mixture may be processed in an acetic acid unit (e.g., having an extractor column vessel, solvent recovery column vessel, and water stripper column vessel) to separate the co-product acetic acid 128 and liquid water 130. In implementations, the water 130 may be recycled for use in the ODH plant 100. For instance, the water 130 discharging from the acetic acid unit may be recycled (as recycle water) for scrubbing process gas in the effluent processing 116 and/or as water (e.g., 120) for the dilution steam in the feed preparation 114), and so on.

After the condensed water and condensed acetic acid are removed from the effluent 108 to the acetic acid unit, the remaining portion of the effluent 108 (minus the condensed liquid) as gas can include ethylene, CO, CO₂, and ethane. The gas may be scrubbed (e.g., with liquid water such as water 130) in a scrubber column vessel to remove residual acetic acid vapor and residual water vapor from the gas. The gas may be subjected to separations to remove CO and CO₂. The gas may sent through a C2 splitter (ethane/ethylene splitter) (a distillation column vessel having distillation trays) to separate the ethane from the gas to give the product ethylene 126.

The equipment 132 in the effluent processing 116 can include, for example, a the heat exchanger for condensing water and acetic acid from the effluent, a flash drum vessel for separating the condensed water and the condensed acetic acid as a mixture from the effluent 106, and an acetic acid unit (e.g., having an extractor column vessel) to process the mixture to separate the co-product acetic acid 128 and liquid water 130. The equipment 132 can include the aforementioned scrubber vessel and a process gas compressor (a mechanical compressor) to increase pressure of the gas having the ethylene from the effluent 108. Other configurations and alternate equipment are applicable.

In some implementations, the equipment 132 can include vessels for separating CO and CO₂ (and other light components) from the gas, as well as the C2 splitter. In other implementations, the equipment 132 does not include such equipment, and the product stream 126 is an intermediate product stream having the ethylene and ethane sent for further processing. Again, other configurations are application. For examples of processing effluent, see WO Published Patent Application No. WO 2022/229847 entitled “Integration for Processing Effluent of Oxidative Dehydrogenation (ODH) Reactor”, which is incorporated by reference herein in its entirety.

The acetic acid unit can be a significant consumer of energy in the ODH plant affected by the amount of dilution steam. The presence of more dilution steam in the effluent 108 (and that is condensed with the acetic acid) can mean more heating (e.g., at steam reboiler on a column) and more cooling (e.g., at an overhead condenser on a column) in the acetic acid unit.

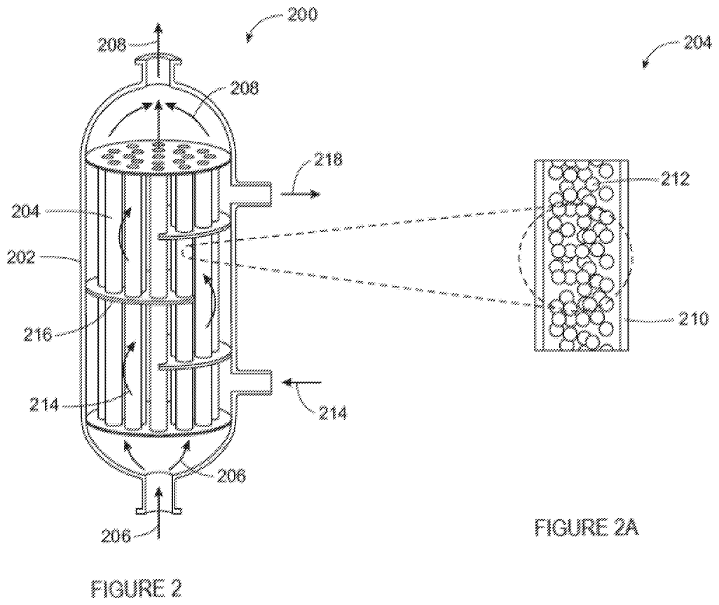

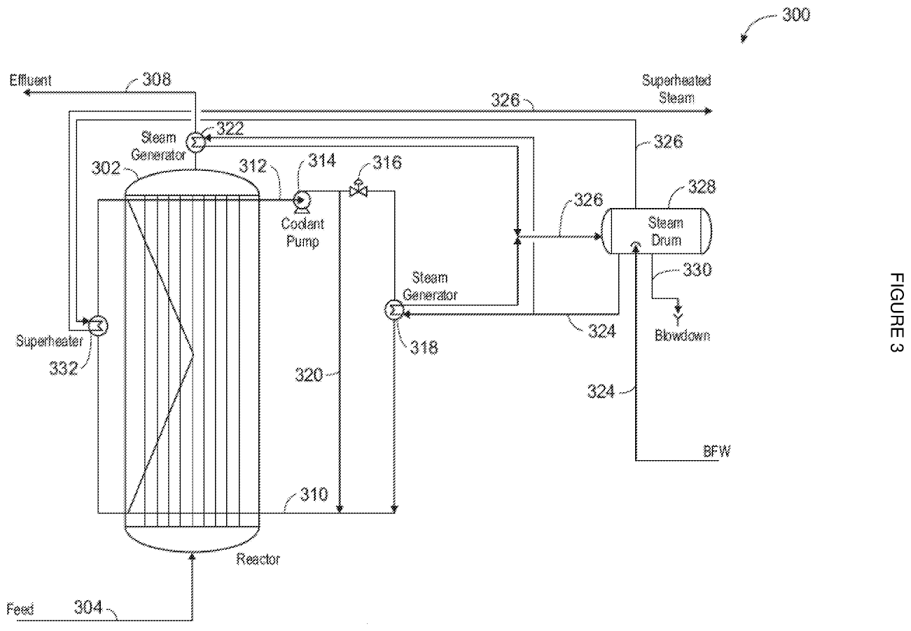

FIG. 2 is a representation of an example multi-tubular fixed bed reactor 200 that is a vessel. The vessel is typically a cylindrical vessel having elliptical or semi-elliptical heads. The vessel may have a vertical orientation (as depicted) or a horizontal orientation. The vessel may be a pressure vessel designed and configured (e.g., with adequate wall thickness) to be subjected to an internal pressure up to a specified pressure (design pressure) greater than ambient pressure (atmospheric pressure). A pressure vessel may be rated to hold a fluid up to the design pressure. In operation, the operating pressure in a pressure vessel may generally be maintained less than the design pressure. A pressure vessel may be constructed per a formal standard or code, such as the American Society of Mechanical Engineers (ASME) Boiler & Pressure Vessel Code (BPVC) or the European Union (EU) Pressure Equipment Directive (PED). For an ODH reactor as a multi-tubular fixed bed reactor, the reactor vessel and the tubes may be metal, such as steel (e.g., stainless steel).

The multi-tubular fixed bed reactor 200 includes a tube side and a shell side. The tube side may be the process side (for conversion in a flowing reaction mixture of process feed into product). The shell side may be the utility side (for flow of a heat transfer fluid). The shell side can be similar in effect to a vessel jacket, but may be similar or analogous to a shell side of a shell-and-tube heat exchanger.

The multi-tubular fixed bed reactor 200 has a shell 202 and a tube bundle, which may be similar to a shell-and-tube heat exchanger, except that the tubes 204 have a fixed bed of catalyst. The tube bundle is multiple tubes 204 generally aligned in parallel. In the tube bundle, the tubes 204 may be supported and held in place as parallel by a tube sheet (e.g., a plate). The tube sheet may be a circular plate that is perforated so that the tubes fit through the perforated openings.

The shell 202 wall may be the vessel wall. The volume of the shell 202 around the longitudinal length of the tubes 204 may be characterized as the shell side of the reactor 202. The shell side may be for flow of heat transfer fluid around the tubes 204. The volume inside the tubes 204 may be characterized as the tube side of the reactor 202. FIG. 2A is an exploded view of a tube 204 having a tube wall 210 and catalyst 212 disposed in the tube 204. Additionally, in FIG. 2, the longitudinal end portions of the vessel may be considered as the tube side. In particular, the longitudinal end portions of the vessel in which the process feed 206 is introduced to the tubes 204 and the process product 208 is discharged from the tubes 204, respectively, may be characterized as the tube side of the reactor 202.

In FIG. 2, the tubes 204 can be filled (at least partially) with catalyst 212. The catalyst 212 may be a fixed bed of catalyst in the tubes 204. The catalyst 212 may facilitate conversion of the feed 206 into the product 208 discharged from the reactor. In operation, the provision (supply) of the feed 206 to the tube side may give a reaction mixture flowing through the tubes 204 and in which reaction(s) in the reaction mixture are promoted (enabled, advanced) by the catalyst 212. The process feed 206 may enter the vessel through a vessel inlet (e.g., inlet nozzle) to the tube side. The process product 208 may discharge from the vessel through a vessel outlet (e.g., outlet nozzle) from the tube side.

The process feed 206 enters a bottom portion of the vessel. The process product 208 discharges from a top portion of the vessel. However, the reactor 200 can be configured for the process feed 206 to enter the top portion of the vessel and the process product 208 to discharge from the bottom portion of the vessel.

A heat transfer fluid 214 may enter the vessel (to the shell side) through a vessel inlet (e.g., inlet nozzle) to flow through the shell side around the tubes 204. For instances of the reaction on the tube side as endothermic, the heat transfer fluid 214 may be a heating medium to provide heat for the endothermic reaction. For implementations of the reaction on the tube side as exothermic, the heat transfer fluid 214 may be a cooling medium (coolant) and in which the heat transfer fluid 214 removes the heat of reaction.

The heat transfer fluid 214 provided to the reactor 200 and that enters the shell side may be labeled as heat-transfer fluid supply. The heat transfer fluid 214 may flow around the tubes 204. Therefore, the heat transfer fluid 214 may be subjected to heat exchange with the process reaction mixture and catalyst 212 on the tube side. The shell side may include baffle(s) 216 to generate more turbulent flow of the heat transfer fluid 214 to improve (increase) heat transfer between the heat transfer fluid 214 and the tube side.

The heat transfer fluid 218 may discharge from the reactor 200 from the shell side, such as through a vessel outlet (e.g., outlet nozzle). The heat transfer fluid 218 that discharges from the reactor 200 from the shell side may be labeled as heat-transfer fluid return sent (as return) to a heat-transfer fluid supply system. The composition of the heat transfer fluid 218 that discharges from the reactor 200 may be the same as the composition of the heat transfer fluid 214 supplied to the reactor 200. The discharged heat-transfer fluid 218 may have the same composition but a different temperature than the supplied heat-transfer fluid 214 due to the heat exchange with the reaction mixture (and catalyst 212) on the tube side.

The flow of the heat transfer fluid 214 through the shell side is in a co-current flow direction with the flow of the reaction mixture on the tube side. However, the flow of the heat transfer fluid 214 through the shell side can instead be in a counter current flow direction with the flow of the reaction mixture on the tube side. For instance, in the illustrated implementation, the reactor 200 can be configured with the inlet for the heat transfer fluid 214 supply on the upper portion of the vessel and the outlet for the heat transfer fluid 218 return on the lower portion of the vessel.

For an ODH reactor as a multi-tubular fixed bed reactor, the heat transfer fluid 214 may typically be a coolant because the ODH reaction is exothermic. Further, the ODH reactor may have more than one cooling section. For instance, a baffle 216 or plate may extend fully across the shell side dividing the shell side into more than one section. Each shell-side section may have an inlet for heat transfer fluid 214 supply and an outlet for heat transfer fluid 218 return. Each shell-side section may be labeled as a cooling section that maintains the reaction mixture and catalyst 212 on the tube side at a respective specified temperature (a respective isotherm temperature).

Implementations of the ODH reactor 110, 112 of FIG. 1 can be analogous to the reactor 200. For an ODH reactor, the number of tubes 204 in the tube bundle can range in the thousands (e.g., 30,000 tubes 204). For an ODH reactor, the process feed 206 can be the feed 104 (FIG. 1). The process feed 206 can be the effluent discharged from an upstream ODH reactor if more than one ODH reactor is employed. The process product 208 can be the effluent 108 (FIG. 1). The process product 208 can be effluent discharged as feed to a downstream ODH reactor if more than one ODH reactor is employed.

As mentioned, for an ODH reactor, the heat transfer fluid 214 may typically be a coolant because the reactions on the tube side are generally exothermic. Heat transfer occurs from the reaction mixture and catalyst 212 on the tube side through the tube wall 210 to the heat transfer fluid 214 flowing through the shell side. The heat transfer fluid 214 as coolant for an ODH reactor can be, for example, steam, water (including pressurized or supercritical water), oil, or molten salt, and so forth. Molten salt may be selected as the coolant due to relatively high stability of the molten salt and due to relatively high heat-transfer coefficient values provided by molten salt at the temperature (e.g., 300-500° C.) of operation of the ODH reactor. Water (critical point is 374° C. and 22,064 kPa) or very high pressure steam employed as the heat transfer fluid 214 would give relatively high pressure on the shell side leading to thicker walls of the shell (vessel).

Embodiments of the present techniques may include specifying the diameter (e.g., nominal diameter, outside diameter, or inside diameter) of the tubes 204 for an ODH reactor to give adequate velocity of the reaction mixture through the tubes 204 to advance a sufficient Reynolds number (Re) to give a satisfactory heat transfer coefficient. The flow hydraulics of the reaction mixture in the tubes 204 can affect heat transfer from the reaction mixture and ODH catalyst 212 to the coolant on the shell side. As discussed, improved heat transfer can disfavor formation of carbon monoxide and carbon dioxide in the reaction mixture flowing through the tubes 204. Embodiments can include configuring the ODH reactor to have the tubes 204 at or less than a specified diameter in response to specifying increasing ethylene selectivity (and thereby increasing the ethylene selectivity).

For typical operating conditions and capacities of an ODH reactor, the tube 204 diameter [e.g., nominal diameter, outside diameter (OD), or inside diameter (ID)] for an ODH reactor may be specified, for example, in the ranges of 0.75 inch to 2.0 inch, 0.75 inch to 1.5 inch, or 0.75 inch to 1.25 inch. Diameters less than 0.75 inch can give difficulty in loading catalyst 212 to the tubes 204 (filling or placing the catalyst 212 into the tubes 204) in implementations. However, tube 204 diameters (for an ODH reactor) less than 0.75 inch can be accommodated in certain implementations. In examples, tube 204 diameters (for an ODH reactor) greater than 1.25 inch or greater than 1.5 inch can give inadequate velocity of the reaction mixture through the tubes 204 leading to poor heat transfer and thus increased formation of carbon dioxide and carbon monoxide. However, tube 204 diameters (for an ODH reactor) greater than 1.5 inch can be accommodated in certain implementations. The Examples below and associated simulations considered a 1-inch tube as OD is 1 inch, tube wall thickness is 0.083 inch, and ID is 0.834 inch; a 1.25-inch tube as OD is 1.25 inch, tube wall thickness is 0.120 inch, and ID is 1.01 inch; and a 1.5-inch tube as OD is 1.5 inch, tube wall thickness is 0.134 inch, and ID=1.232 inch. For a hypothetical example, a 2-inch tube may be, for instance, OD is 2 inch, tube wall thickness is 0.188 inch, and ID is 1.624 inch.

In some implementations, the ODH reactor may have a supplemental tube bundle for superheating steam. In operation, the supplemental tube bundle receives steam (e.g., saturated steam). The steam flows through the tubes of the supplemental tube bundle and is heated by the heat transfer fluid 214. The steam discharges as superheated from the reactor 200 from the supplemental tube bundle. The supplemental tube bundle may be in the reactor vessel adjacent the primary tube bundle. On the other hand, the supplemental tube bundle may have a dedicated shell adjacent to or mounted on the reactor 200 vessel and the dedicated shell coupled to the shell side of the reactor 200 for flow of the heat transfer fluid 214. The supplemental dedicate shell may receive a slipstream of coolant from the main shell.

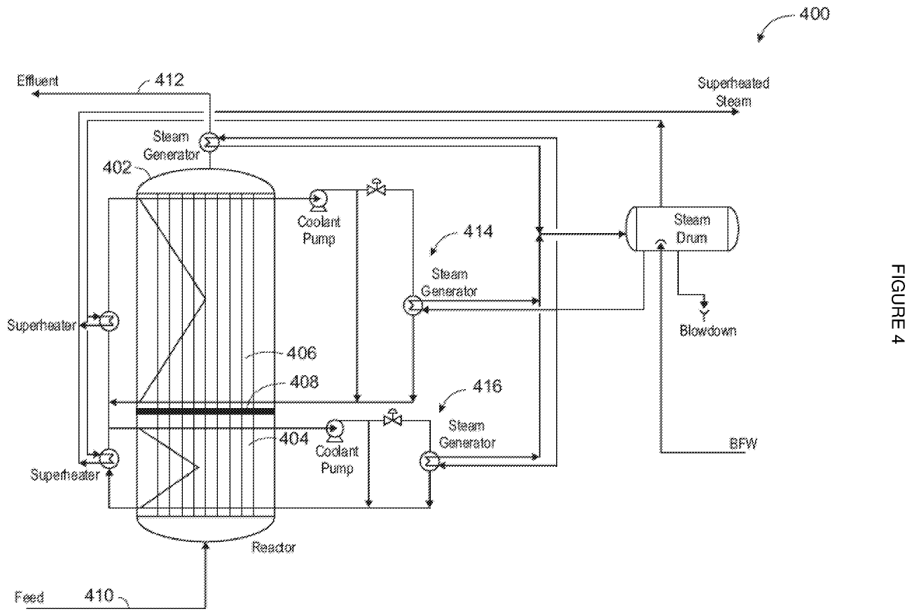

Options of ODH reactor systems are given. The example of Configuration 2 presented below may be a base case. Configurations 1 and 3-13 presented may be generally compared to Configuration 2 as a baseline case. However, the present techniques are not limited to the various options and configurations as tabulated or characterized. Instead, the various configurations including Configurations 1-13 are given as examples. FIGS. 3-12 may be presented with respect to each other and some include incremental differences with respect to each other. For a description of equipment and operation in a given figure of FIGS. 3-12, see also the discussion of the other figures of FIGS. 3-12.

FIG. 3 is an ODH reactor system 300 that may be the ODH reactor system 102 of FIG. 1. For comparison of examples of ODH reactor systems disclosed herein, an implementation of the ODH reactor system 300 is labeled as Configuration 1.

The ODH reactor system 300 includes an ODH reactor 302 that is a multi-tubular fixed bed reactor. The reactor 302 is depicted with a symbol of multiple vertical lines representing collectively a shell side and tube side in a reactor vessel. The two diagonal lines are a symbol for flow of coolant through the shell side for heat exchange between the shell side and the tube side, and indicate a cooling section. The reactor 302 has one cooling section. A coolant system external to the reactor 302 is associated with the cooling section. A cooling section may remove the heat of reaction of the exothermic reaction on the tube side to give a reactor isotherm temperature in which the process reaction mixture and ODH catalyst on the tube side are maintained at a constant temperature (e.g., in a range of 300° C. to 500° C.).

The ODH reactor 302 as a multi-tubular fixed bed reactor includes a tube bundle having multiple tubes. In one implementation for FIG. 3, the diameter (e.g., nominal diameter, outside diameter, or inside diameter) of each tube is 1 inch. The inside (interior volume) of the tubes may be the tube side. The shell side may be the volume in the reactor vessel along the tube bundle around the tubes. In implementations, the vessel wall along the straight side of the vessel may be the shell wall. In operation, heat transfer may occur from the tube side (inside the tubes) through the tube wall of each tube to the shell side. In particular, heat transfer may occur from the reaction mixture (flowing in the tubes) and ODH catalyst in the tubes through the tube wall to coolant flowing through the shell side. In implementations, the coolant is molten salt.

ODH catalyst (e.g., as a fixed bed) may be disposed inside the tubes. The term “ODH catalyst” as used herein may be catalyst known for ODH of ethane. The ODH catalyst may give an ODH reaction that dehydrogenates ethane to ethylene and in which acetic acid as a byproduct may be formed. A low-temperature ODH catalyst may be beneficial. One non-limiting example of an ODH catalyst that may be utilized in an ODH reactor is a low-temperature ODH catalyst that includes molybdenum (Mo), vanadium, tellurium (Te), niobium, and oxygen (O), wherein the molar ratio of molybdenum to vanadium is from 1:0.12 to 1:0.49, the molar ratio of molybdenum to tellurium is from 1:0.01 to 1:0.30, the molar ratio of molybdenum to niobium is from 1:0.01 to 1:0.30, and oxygen is present at least in an amount to satisfy the valency of any present metal elements. The molar ratios of molybdenum, vanadium, tellurium, niobium can be determined by inductively coupled plasma mass spectrometry (ICP-MS). The catalyst may be labeled as low temperature ODH catalyst in providing for the ODH reaction at less than 450° C., less than 425° C., less than 400° C., or less than 375° C., or in a range, for example, of 300° C. to 500° C.

The reactor 302 vessel includes a feed inlet to receive feed 304 (e.g., analogous to feed 104 of FIG. 1 or feed 206 of FIG. 2) into the reactor 302 to the tube side to give a reaction mixture flowing through the tube side. In the illustrated embodiment, the reaction mixture flows upward through the tubes. The feed inlet may be a vessel inlet nozzle coupled to a feed conduit that conveys the feed 304 to the reactor 302. The feed 304 may include ethane, oxygen, and diluent. The diluent may be steam as dilution steam.

As discussed, associated with the ODH reaction that dehydrogenates the ethane to the ethylene, a byproduct formed in the reaction mixture flowing through the tube side may be acetic acid. As further mentioned, byproducts formed in the reaction mixture flowing through the tube side may also include water, CO₂, and CO. Thus, the effluent 308 (e.g., which may be analogous to effluent 108 of FIG. 1 or process product 208 of FIG. 2) discharged from the ODH reactor 302 vessel may include ethylene, acetic acid, water, CO₂, CO, unreacted ethane, and unreacted diluent (which may be water in embodiments).

To remove the heat of reaction from the reaction mixture flowing through the tube side, the reactor system 300 may include a coolant supply system that supplies coolant 310 to the shell side of the reactor 302. The coolant 310 as introduced through an inlet nozzle on the reactor 302 vessel to the shell side may be labeled as coolant supply or cold coolant (e.g., cold salt for the coolant as molten salt). Heat transfer may occur from the reaction mixture and ODH catalyst on the tube side to the coolant 310 flowing through the shell side. The heated coolant 312 may discharge from the reactor 302 (e.g., through a reactor vessel outlet nozzle) from the shell side as coolant return (hot coolant or hot salt for the coolant as molten salt) to the coolant supply system. The coolant supply system may maintain the reaction mixture and ODH catalyst at a specified temperature. The coolant 310 and the heated coolant 312 may generally have the same composition.

The heated coolant 312 typically has a greater temperature than the coolant 310 as supplied. In other words, the coolant experiences a temperature increase (ΔT) across the shell side. The amount of energy (Q) as heat received by the coolant from the tube side may be Q=mcₚΔT, where Q is energy per time [e.g., kilojoules per second (kJ/s) or kilowatts (kW)], m is flow rate in mass per time [e.g., kilograms per second (kg/s)] of the coolant, cₚ is the specific heat capacity [e.g., units of (kJ/kg) (1/° C.)) of the coolant, and ΔT is the temperature increase (e.g., ° C.) of the coolant. Q is the amount of heat received by the coolant to maintain the tube side (reaction mixture, ODH catalyst) at a specified temperature.

As can be seen in the equation Q=mcₚΔT, for a given Q to maintain the tube side at specified temperature, the temperature increase (ΔT) of the coolant and the flow rate (m) of the coolant are inversely proportional. Therefore, specifying a lower temperature increase (ΔT) of the coolant gives a greater flow rate (m) of the coolant through the shell side. Conversely, specifying a higher temperature increase (ΔT) of the coolant gives a lower flow rate (m) of the coolant through the shell side. The temperature increase (ΔT) of the coolant can be specified to give adequate flow rate (m) of the coolant (e.g., to give a sufficient Reynolds number (Re)) to give a satisfactory heat transfer coefficient. The flow hydraulics of the coolant flowing on the shell side along the tubes can affect heat transfer to the coolant from the reaction mixture on the tube side.

The coolant supply system may include a coolant pump 314 (a circulation pump), a control valve 316, and a coolant heat exchanger 318. The coolant pump 314 (e.g., a centrifugal pump) provides motive force of circulation of coolant through the shell side of the reactor 302. The suction (inlet) of the pump 314 receives the heated coolant 312 (the conduit return) as a suction fluid. The pump 314 discharges the heated coolant 312 through the control valve 316 and the coolant heat exchanger 318.

The coolant heat exchanger 318 (e.g., a shell-and-tube heat exchanger) removes heat from the heated coolant 312 to give the coolant 312 (coolant supply) at a desired (specified) temperature (the inlet temperature of the coolant 310 to the reactor 302). The control valve 316 regulates (modulates, adjusts, maintains, alters) the amount of heated coolant 312 flowed through the heat exchanger 318 to give the coolant 310 supply at the desired temperature. A bypass portion 320 of the heated coolant 312 flows through a bypass conduit bypassing the coolant heat exchanger 318. The total flow rate of the coolant in the circulation through the coolant supply system and the shell side of the reactor 302 may be generally be substantially constant. This flowrate of coolant provided by the pump 314 through the shell side will be lower at a specified higher increase of temperature (ΔT) of the coolant through the shell side. This flowrate of coolant provided by the pump 314 through the shell side will be greater at a specified lower increase of temperature (ΔT) of the coolant through the shell side.

The ODH reactor 302 vessel may have an effluent outlet for discharge of the effluent 308 from the ODH reactor 302. The effluent outlet may be a vessel outlet nozzle that is coupled to an effluent discharge conduit for flow of the discharged effluent 308 from the reactor 302. The temperature of the effluent 308 as discharged may be, for example, in the range of 300° C. to 500° C. commensurate with the operating temperature (e.g., 300° C. to 500° C.) of the ODH reactor 302 vessel.

An effluent heat exchanger 322 is disposed along the effluent discharge conduit. The heat exchanger 322 cools the effluent 308 with water 324 as a cooling medium. For example, the effluent exchanger 322 may cool the effluent 308 to a temperature in the range of 150° C. to 350° C. (or in the range of 200° C. to 300° C.). The heat exchanger 322 may be labeled as a transfer line exchanger (TLE). In the depicted implementation, the heat exchanger 322 is a steam generation heat exchanger to heat (vaporize) water 324 (e.g., liquid water) with the effluent 308 as a heating medium to generate steam 326 from the water 312. Thus, the effluent heat exchanger 322 may characterized as a steam-generation heat exchanger. The water 324 may be heated in the heat exchanger 322 with heat from the effluent 308 to flash (vaporize) the water 324 into the steam 326. In alternate embodiments, the effluent heat exchanger 322 may heat (pre-heat) the water 312 for downstream vaporization of the water into the steam 326. In those embodiments, the effluent heat exchanger 322 may be characterized as a preheater (e.g., BFW preheater). The effluent heat exchanger 322 (steam generation heat exchanger) may be, for example, a shell-and-tube heat exchanger or a fin-type heat exchanger (e.g., with a finned-tube bundle), and so on. The water 324 may be, for example, demineralized water, steam condensate, or boiler feedwater (BFW), and the like. Boiler feedwater may be treated demineralized water. The effluent 308 may be cooled, for example, by an amount in a range of 50° C. to 350° C. across the heat exchanger 322.

The coolant heat exchanger 318 may also be utilized to generate steam 326 from the water 324. Thus, the coolant heat exchanger 318 may be labeled as a steam-generation heat exchanger. As with the effluent heat exchanger 322, the coolant heat exchanger 318 may be, for example, a shell-and-tube heat exchanger or a fin-type heat exchanger (e.g., with a finned-tube bundle), and the like.

In embodiments, one or both of the heat exchangers 318, 322 may heat (pre-heat, not vaporize) the water 324 for vaporization of the water 324 into steam 326 in the steam drum 328. In those embodiments, one or both of the heat exchangers 318, 322 may receive water 324 from the steam drum 328 or from the boiler feedwater (BFW) pump, and discharge heated (pre-heated) water 324 (generally not steam 326) to the steam drum 328.

The reactor system 300 may include a steam drum 328 vessel (e.g., a VHP steam drum) to provide the water 324 to the heat exchangers 318, 322. The steam drum 328 vessel can have a horizontal orientation (as depicted) or a vertical orientation. The steam drum 328 vessel may receive water 324 via a water feed conduit from a pump (not shown) (e.g., a boiler feedwater pump). The steam drum 328 vessel may have a vessel inlet nozzle to receive the water 324. The steam drum 328 may discharge the water 324 through a vessel outlet nozzle to the heat exchangers 318, 322. The motive force for flow of the water 324 from the steam drum 328 to the heat exchangers 318, 322 (and for flow of steam 326 from the heat exchangers 318, 322 to the steam drum 328) may be, for example, by thermosiphon.

In operation, a liquid level of water 324 is maintained in the steam drum 328 vessel, such as via liquid level controls. The liquid level controls may include a level control valve on a discharge conduit from a bottom portion of the steam drum 328 vessel (form the vessel outlet nozzle for flow of the water 324 to the heat exchangers 318, 322), a level sensor at the steam drum 328 vessel, an instrument transmitter to indicate the liquid level as measured by the level sensor to a control system, and control logic in the control system to control the level control valve to maintain the liquid level at a set point.

In some implementations, a small portion of the water 324 (or a slipstream of water 324) as discharged from the steam drum 328 vessel may be sent to blowdown (e.g., sewer), as indicated by reference numeral 330, to prevent or reduce accumulation of impurities (e.g., solids) in the steam drum 328 vessel. The blowdown may be intermittent.

The steam 326 generated by the heat exchangers 318, 322 may be routed to the steam drum 328. The steam drum 328 may have a vessel inlet nozzle to receive the steam 326. The steam 326 generated by the heat exchangers 318, 322 can be low pressure (LP) steam (e.g., 150 pounds per square inch gauge [psig] or less), medium pressure (MP) steam (e.g., in the range of 150 psig to 500 psig), high pressure (HP) steam (e.g., in the range of 500 psig to 900 psig), or very high pressure (VHP) steam (e.g., in the range of 900 psig to 1700 psig), and so forth. For steam that may be HP steam or VHP steam, the notation HP/VHP steam (e.g., in the range of 500 psig to 1700 psig) is employed. In implementations, the steam 326 is HP/VHP steam. The pressure of the steam generated via the steam-generation heat exchanger 310 may be a function of the temperature of the effluent 308 driven by the operating temperature (ODH reaction temperature) of the ODH reactor 302. The operating temperature in the steam drum 328 vessel may be, for example, in the range of 240° C. to 330° C. for the steam 314 as HP/VHP steam.

Higher-pressure steam, such as greater than 500 pounds per square inch gauge (psig) or greater than 900 psig, may typically be more valuable than lower pressure steam, such as less than 500 psig or less than 150 psig. At the steam-generation heat exchangers 318, 322, generation of HP steam or VHP steam may be generally be more beneficial (valuable) than generating MP steam or LP steam and can thus improve economics of the reactor system 300 and associated ODH plant (facility). There may be different applications for the steam 326. The use of the steam by the consumers or customers receiving the steam may depend on the pressure of the steam. In some implementations, higher steam pressures of the produced steam may give more versatility in the integration of the steam within the facility or plant. For instance, HP steam can be utilized to power turbines attached to compressors, while LP steam is typically used for heating purposes, and the like.

In operation of the steam drum 328, the steam 326 that enters the steam drum 328 may discharge overhead as steam 326 from the steam drum 328. Any liquid water 324 that flashes (vaporizes) in the steam drum 328 may discharge overhead as steam 326. The steam 326 that discharges overhead from the steam drum 328 may be saturated steam (or slightly above saturation temperature). The steam drum 328 may have a vessel outlet nozzle on an upper or top portion of the steam drum 328 vessel for discharge of the steam 326. The steam 326 (e.g., saturated steam or near saturation) may discharge overhead from the steam drum 328 into a conduit for distribution to users (e.g., at the ODH plant facility).

The ODH reactor system 300 may superheat the steam 326 before distribution of the steam 326 to users. In particular, the steam 326 may flow through a superheater 332 heat exchanger prior to distribution. The steam 326 that discharges from the superheater 332 heat exchanger is the steam 326 as superheated by the superheater 332. The heating medium for the superheater 332 is the coolant flowing through the shell side of the reactor 302. The symbol depicted for the superheater 332 is a symbol for a heat exchanger. The heat-exchanger symbol for the superheater 332 may be representative of a heat exchanger (e.g., shell-and-tube heat exchanger or a plate-fin type heat exchanger) disposed external to the reactor 302 and in which a slipstream of coolant is routed from the shell side of the reactor 302 through the superheater 332.

The heat-exchanger symbol for the superheater 332 may be representative of an add-on or supplemental tube bundle (with dedicated surrounding shell) having a relatively small number of tubes (not having ODH catalyst). The add-on tube bundle may supplement the tube side of the reactor 302 and in which the steam 326 is flowed through the tubes of the add-on tube bundle (a second tube bundle). Coolant (a slipstream) from the main shell side of the reactor 302 flows through the shell side of the add-on tube bundle to heat (superheat) the steam 326. The add-on tube bundle with the dedicated shell can be mounted to the reactor 302 vessel and the dedicated shell coupled to the shell side of the reactor 302 for flow of the coolant. The supplemental tube bundle can be a tube bundle (another tube bundle or second tube bundle) inside the reactor 302 vessel adjacent to the main tube bundle.

In operation, the add-on or supplemental tube bundle receives steam 326 (e.g., saturated steam) through a steam inlet nozzle. The steam 326 flows through the tubes of the add-on tube bundle and is heated by the coolant from the shell of the reactor 302. The steam 326 discharges as superheated from the reactor 302 from the add-on tube bundle. The add-on or supplemental tube bundle (with dedicated shell) mounted to the reactor 302 vessel may have an outlet (superheated steam outlet nozzle) for discharge of the superheated steam 326.

The tubes of the add-on or supplemental tube bundle may generally be parallel with the tubes of the main tube bundle in the main shell of the reactor 302. The add-on tubes may typically be separate from the main bundle. The add-on tubes may share the shell coolant. For the add-on tube bundle mounted on the exterior of the reactor 302 vessel, a slipstream of main shell coolant may flow as add-on shell coolant.

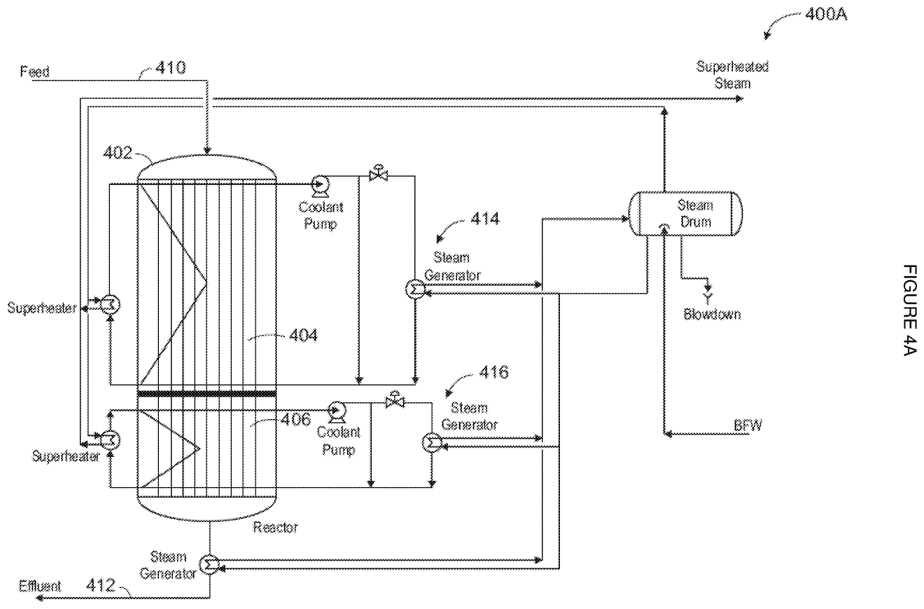

FIG. 4 is an ODH reactor system 400 that is similar to the ODH reactor system 300 of FIG. 3, except that the sole ODH reactor 402 has two cooling sections instead of a single shell or single cooling section as in the ODH reactor 302. Refer to FIG. 3 for a discussion of similar or same equipment and operation. FIG. 4A is an ODH reactor system 400A that is the same as the ODH reactor system 400 of FIG. 4, except that the coolant flow is in a countercurrent (instead of co-current) flow direction with respect to the reaction mixture flowing through the tube side. The ODH reactor systems 400 and 400A may each be the ODH reactor system 102 of FIG. 1. For comparison of examples of ODH reactor systems disclosed herein, implementations of the ODH reactor systems 400 and 400A provide a basis for Configurations 2-5. In an example analysis, Configuration 2 is selected as a base case in a comparison of Configuration 1-13.

The ODH reactor 402 has a first cooling section 404 and a second cooling section 406 separated by a barrier 408 on the shell side of the ODH reactor 402. For “first” cooling section and “second” cooling section presented herein in the various embodiments, the second cooling section is operationally downstream of the first cooling section in the flow direction of the reaction mixture in the tubes. The flow of the coolant through the shell of the first cooling section can be co-current or counter current with respect to the reaction mixture flowing through the tubes. The flow of the coolant through the shell of the second cooling section can be co-current or counter current with respect to the reaction mixture flowing through the tubes.

The barrier 408 may be, for example, a metal plate that seals the shell side of the first cooling section 404 from the shell side of the second cooling section 406. Therefore, in operation, the coolant in the first cooling section 404 is separate from the coolant in the second cooling section 408. The barrier 408 may have perforations or other openings for the tubes of the tube bundle to pass through the barrier 408, and therefore for the reaction mixture to flow through both cooling sections 404, 406.

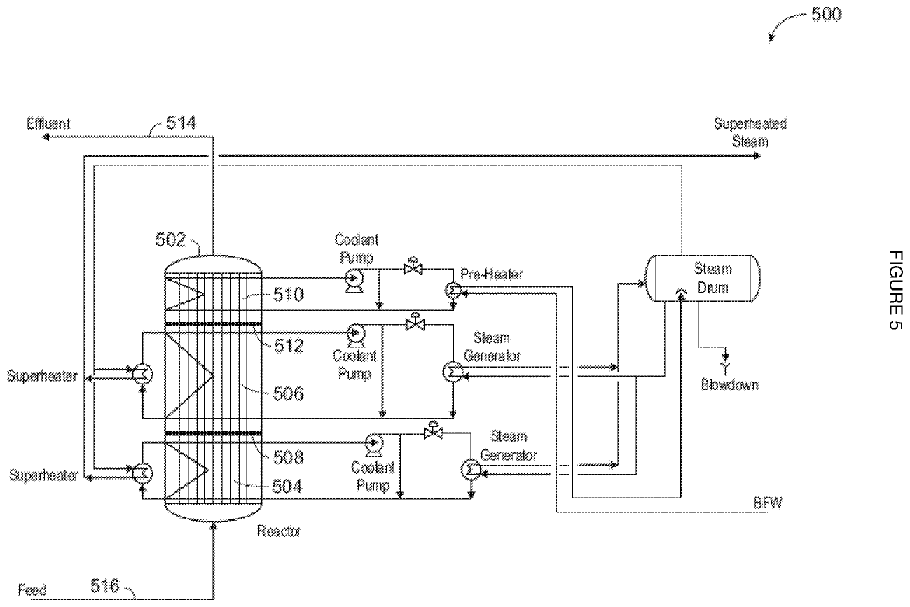

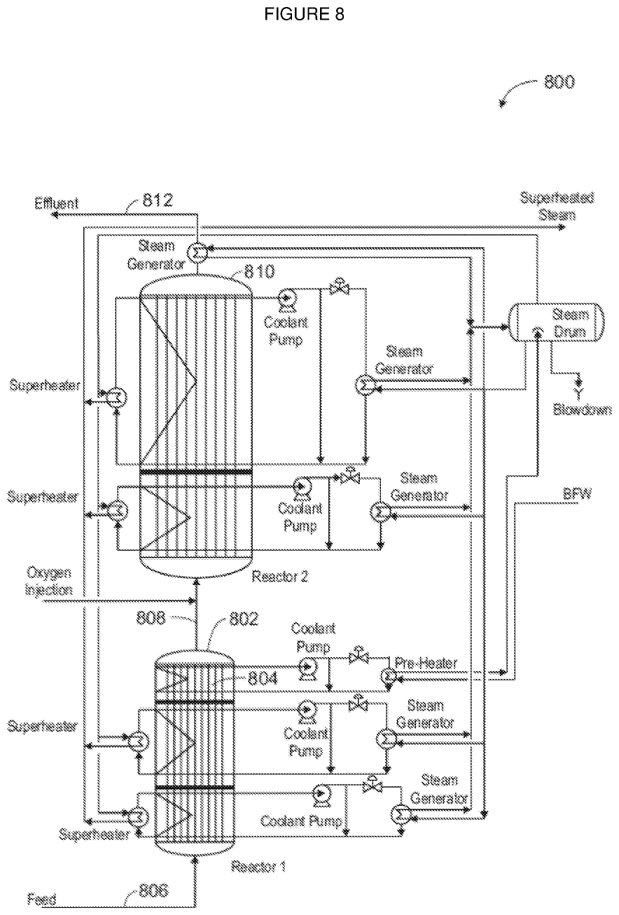

A cooling section in an ODH reactor may be labeled as a catalyst cooling section for cooling sections in which the tubes have catalyst (ODH catalyst). In FIG. 4, both the first cooling section 404 and the second cooling section are each a catalyst cooling section. A catalyst cooling section may be utilized to cool both the reaction mixture and the ODH catalyst on the tube side. For cooling sections in which the tubes do not have catalyst (ODH catalyst), the cooling section may be labeled as a non-catalyst cooling section in which the cooling section is utilized to cool the reaction mixture on the tube side. For examples of employment of a non-catalyst cooling section, see cooling section 510 of FIG. 5 and cooling section 804 of FIG. 8. For non-catalyst cooling sections, inert particles may be disposed in the tubes. In implementations, employment of a non-catalyst cooling section can be characterized as a coolant quench of the reaction mixture on the tube side. For instances of the coolant as molten salt, the coolant quench can be called a salt quench.

The feed 410 to the reactor 402 as introduced to the tubes (tube side) may give the initial reaction mixture in the first cooling section 404. The feed 410 may be analogous to the feed 104 of FIG. 1. The reaction mixture flows through the tubes. The reaction mixture flowing in the tubes flows from the first cooling section 404 through the barrier 408 into the second cooling section 406. The reaction mixture as discharged from the tubes (tube side) of the second cooling section 406 may give the effluent 412 of the ODH reactor 402. The effluent 412 may be analogous to the effluent 108 of FIG. 1.

The first cooling section 404 and the second cooling section 406 may give a respective isotherm temperature for the reactor 402. In particular, each cooling section 404, 406 may give or facilitate a respective constant temperature of the reaction mixture and ODH catalyst on the tube side by removing the heat of reaction (exothermic).

As discussed (e.g., with respect to FIG. 3), a coolant supply system for an ODH reactor may include a coolant pump (e.g., centrifugal pump) to receive the coolant from the shell side of the ODH reactor and to provide motive force for the circulation of coolant through the shell side. The coolant pump may be configured to give a specified capacity (circulation flow rate of the coolant) based on a specified temperature increase for the coolant through the shell side. The specifying of the temperature increase as lower (e.g., in range of 2° C. to 8° C.) gives configuration of the coolant pump to provide greater flow rate advantageous to increasing efficiency of heat transfer from the tube side to the shell side. The coolant supply system may include a coolant heat exchanger to cool at least a portion of the coolant to give a desired or specified coolant supply temperature to the shell side. The coolant supply system may include a control valve (e.g., flow control valve) to regulate the amount of the coolant in the circulation that is routed through the coolant heat exchanger (and thus the amount of the coolant that bypasses the coolant heat exchanger).

A first coolant supply system 414 circulates coolant (e.g., molten salt) through the shell side in the first cooling section 404. The first coolant system 414 provides coolant supply to the first cooling section 404 and receives coolant return from the first cooling section 404. A second coolant supply system 416 circulates coolant (e.g., molten salt) through the shell side in the second cooling section 406. The second coolant system 416 provides coolant supply to the second cooling section 406 and receives coolant return from the second cooling section 406.

The coolant heat exchanger in the first coolant supply system and the coolant heat exchanger in the second coolant supply system may each receive water (e.g., BFW) from the steam drum and heat the water (with coolant as the heating medium) to vaporize the water to give steam (e.g., HP/VHP steam). Similarly, an effluent heat exchanger receives water from the steam drum to cool the effluent 412 and therefore vaporizes the water (with effluent 412 as heating medium) to give steam. As discussed, the steam may discharge from the steam drum for distribution to users. As also discussed, the steam may be superheated in a heat exchanger (e.g., as in 332 of FIG. 3) associated with a cooling section. In FIG. 4, there are two such heat exchangers associated with the first cooling section 404 and the second cooling section 406, respectively.

The heating medium for the two superheater heat exchangers is the coolant flowing through the shell side of the first cooling section 404 and the second cooling section 406, respectively. The superheater heat exchangers may each be: [1] a heat exchanger (e.g., shell-and-tube heat exchanger or a plate-fin type heat exchanger) disposed external to the reactor 402 and in which a slipstream of coolant is routed from the shell side through the superheater heat exchanger; [2] a second tube bundle having a few tubes (not having ODH catalyst) adjacent to the main tube bundle in the reactor 402 and in which the steam is flowed through those tubes such that the coolant flowing through the shell side in each cooling section 404, 406 heats (superheats) the steam; and/or [3] a mounted add-on (supplemental) tube bundle (second tube bundle) (with dedicated surrounding shell) in which the steam flows through the tubes (not having ODH catalyst) and coolant (e.g., a slip stream) from the main shell side of the reactor 402 flows through the shell side of the add-on tube bundle to heat (superheat) the steam.

In general, with steam generation heat exchangers (e.g., the two coolant heat exchangers and the effluent heat exchanger in FIG. 4), the heat exchangers can be configured to heat (pre-heat, not vaporize) the water for vaporization of the water into steam in the steam drum. In those embodiments, the heat exchangers may receive water from the BFW pump and discharge heated (pre-heated) water (generally not steam) to the steam drum.

The second cooling section 406 is disposed operationally downstream of the first cooling section 404 in the direction of flow of the reaction mixture on the tube side. Thus, the concentration of oxygen in the reaction mixture in the first cooling section 404 may generally be greater than in the reaction mixture in the second cooling section 406. Therefore, with greater availability of oxygen in the first cooling section 404, the reactions in the reaction mixture may be more susceptible to including reactions giving carbon monoxide and carbon dioxide. In contrast, oxygen is less concentrated (less available) in the second cooling section 406. Therefore, the reactions in the reaction mixture in the second cooling section 404 may be less susceptible to being reactions giving carbon monoxide and carbon dioxide. An advantage of having two cooling sections for an ODH reactor may be that the tube side can be operated at lower temperature in the first cooling section 404 to reduce reactions forming carbon monoxide and carbon dioxide in the presence of higher concentrations of oxygen. For instance: (1) the reaction mixture and ODH catalyst on the tube side in the first cooling section 404 can be maintained at a temperature in the ranges of 300° C. to 400° C. or 300° C. to 450° C.; and (2) the reaction mixture and ODH catalyst on the tube side in the second cooling section 406 can be maintained at a temperature in a range of 350° C. to 500° C.

Again, the cooling sections 404, 406 and associated coolant supply systems remove the heat of reaction (exothermic) generated in the reaction mixture of the tube side. The amount of heat removed can be altered to give the specified isotherm temperature. The amount of cooling flowing through each cooling section can be altered to give a specified temperature increase (e.g., in range of 2° C. to 8° C.) of coolant through the cooling sections 404, 406.

FIG. 5 is an ODH reactor system 500 that is similar to the ODH reactor system 400 of FIG. 4, except that the sole ODH reactor 502 has [1] a third cooling section (a non-catalyst cooling section) that can be characterized as a coolant (salt) quench, and [2] no effluent heat exchanger to cool the effluent is employed. Refer to FIGS. 3-4 for a discussion of similar or same equipment and operation. The ODH reactor system 500 may be the ODH reactor system 102 of FIG. 1. For comparison of examples of ODH reactor systems disclosed herein, an implementation of the ODH reactor system 500 provides a basis for Configuration 6.

The ODH reactor 502 has a first cooling section 504 and a second cooling section 506 separated by a barrier 508 on the shell side of the ODH reactor 502. As discussed, the barrier 508 may be, for example, a metal plate that seals the shell side of the first cooling section 504 from the shell side of the second cooling section 506. Therefore, in operation, the coolant (e.g., molten salt) in the first cooling section 504 is separate from the coolant (e.g., molten salt) in the second cooling section 506. The barrier 508 may have perforations or other openings for the tubes of the tube bundle to pass through the barrier 508, and therefore for the reaction mixture to flow through both cooling sections 504, 506.

The ODH reactor 502 has a third cooling section 510 separated from the second cooling section 506 by a barrier 512 on the shell side of the ODH reactor 502. Similar to the barrier 508, the barrier 512 (e.g., a metal plate with openings for the tubes) seals the shell side of the third cooling section 510 from the shell side of the second cooling section 506. Therefore, in operation, the coolant (e.g., molten salt) in the third cooling section 510 is separate from the coolant in the second cooling section 506.

The reaction mixture flows on the tube side from the second cooling section 508 through the third cooling section 510. The third cooling section 510 is a non-catalyst cooling section in that the tubes in the third cooling section 510 do not have catalyst (but may have inert particles disposed therein). Therefore, reactions in the third cooling section 510 may be limited. A purpose of the cooling section 510 may be to cool the reaction mixture flowing through the tubes in the third cooling section 510 before the reaction mixture discharges as the effluent 514. Thus, in implementations, the third cooling section 510 may be implemented in lieu of an effluent heat exchanger that cools the effluent 514. The effluent 514 may be analogous to the effluent 108 of FIG. 1. In implementations, the third cooling section 510 may cool the effluent 514 to a temperature in a range of 150° C. to 350° C. (or in the range of 200° C. to 300° C.).

The feed 516 to the reactor 502 as introduced to the tubes (tube side) may give the initial reaction mixture in the first cooling section 504. The feed 516 may be analogous to the feed 104 of FIG. 1. The reaction mixture flows in the tubes. The tubes are routed through the barriers 508 and 512. The barriers 508 and 512 are not inside the tubes. The reaction mixture flows in the tubes across the barrier 508 from the first cooling section 504 to the second cooling section 506. The reaction mixture flows in the tubes across the barrier 512 from the second cooling section 506 to the third cooling section 510. The reaction mixture as discharged from the tubes (tube side) of the third cooling section 510 may give the effluent 514 of the ODH reactor 502.

The cooling sections 504, 506 may each give a respective isotherm temperature for the reactor 502. In particular, the cooling sections 504, 506 may give or facilitate a respective constant temperature of the reaction mixture and ODH catalyst on the tube side. As mentioned, the third cooling section 510 may cool reaction mixture for discharge as the effluent 514 having a temperature in a range of 150° C. to 350° C. (or in the range of 200° C. to 300° C.). The third cooling section 510 may be in lieu of TLE and thus act more literally as a HEX, though the third cooling section 510 can approach isothermal on the coolant (molten salt) side but the coolant can have rising temperature with less molten salt coolant circulation. The third cooling section 510 (e.g., in lieu of TLE) may reduce the tube-side reaction mixture temperature, for example, from 450° C. to 150° C.-200° C.

As discussed (e.g., with respect to FIGS. 3-4), a coolant supply system for an ODH reactor may include a coolant pump (e.g., centrifugal pump) to receive the coolant from the shell side of the ODH reactor and to provide motive force for the circulation of coolant through the shell side. The coolant pump may be configured to give a specified capacity (circulation flow rate of the coolant) based on a specified temperature increase for the coolant through the shell side. The specifying of the temperature increase as lower (e.g., in range of 2° C. to 8° C.) gives configuration of the coolant pump to provide greater flow rate advantageous to increasing efficiency of heat transfer from the tube side to the shell side. The coolant supply system may include a coolant heat exchanger to cool at least a portion of the coolant to give a desired or specified coolant supply temperature to the shell side. The coolant supply system may include a control valve (e.g., flow control valve) to regulate the amount of the coolant in the circulation that is routed through the coolant heat exchanger (and thus the amount of the coolant that bypasses the coolant heat exchanger).

In FIG. 5, a first coolant supply system circulates coolant (e.g., molten salt) through the shell side in the first cooling section 504. The first coolant supply system provides coolant supply to the first cooling section 504 and receives coolant return from the first cooling section 504. A second coolant supply system circulates coolant (e.g., molten salt) through the shell side in the second cooling section 506. The second coolant supply system provides coolant supply to the second cooling section 506 and receives coolant return from the second cooling section 506. A third coolant supply system circulates coolant (e.g., molten salt) through the shell side in the third cooling section 510. The third coolant supply system provides coolant supply to the third cooling section 510 and receives coolant return from the third cooling section 510.

The coolant heat exchanger in the first coolant supply system and the coolant heat exchanger in the second coolant supply system may each receive water (e.g., BFW) from the steam drum and heat the water (with coolant as the heating medium) to vaporize the water to give steam (e.g., HP/VHP steam) to the steam drum. The coolant heat exchanger in the third coolant supply system heats water (e.g., BFW) with coolant as heating medium to send the heated water to the steam drum.

As discussed, steam may discharge from the steam drum for distribution to users. As also discussed, the steam may be superheated in a heat exchanger (e.g., as in 332 of FIG. 3) associated with a cooling section prior to distribution of the steam to users. In FIG. 5, there are two such heat exchangers associated with the first cooling section 504 and the second cooling section 506, respectively.

The heating medium for the two superheater heat exchangers is the coolant flowing through the shell side of the first cooling section 504 and the second cooling section 506, respectively. The superheater heat exchangers may each be: [1] a heat exchanger (e.g., shell-and-tube heat exchanger or a plate-fin type heat exchanger) disposed external to the reactor 502 and in which a slipstream of coolant is routed from the shell side through the superheater heat exchanger; [2] a second tube bundle having a few tubes (not having ODH catalyst) adjacent to the main tube bundle in the reactor 502 and in which the steam is flowed through those tubes such that the coolant flowing through the shell side in each cooling section 504, 506 heats (superheats) the steam; and/or [3] a mounted add-on (supplemental) tube bundle (second tube bundle) (with dedicated surrounding shell) in which the steam flows through the tubes (not having ODH catalyst) and coolant (e.g., a slip stream) from the main shell side of the reactor 402 flows through the shell side of the add-on tube bundle to heat (superheat) the steam.

In general, with steam generation heat exchangers (e.g., the two coolant heat exchangers in FIG. 5), the heat exchangers can be configured instead to heat (pre-heat, not vaporize) the water for vaporization of the water into steam in the steam drum. In those embodiments, the heat exchangers may receive water from the BFW pump and discharge heated (pre-heated) water (generally not steam) to the steam drum.

The second cooling section 506 is disposed operationally downstream of the first cooling section 504 in the flow direction of the reaction mixture on the tube side. Thus, the concentration of oxygen in the reaction mixture in the first cooling section 504 may generally be greater than in the reaction mixture in the second cooling section 506. Therefore, with greater availability of oxygen in the first cooling section 504, the reactions in the reaction mixture may be more susceptible to including reactions giving carbon monoxide and carbon dioxide. In contrast, oxygen is less concentrated (less available) in the second cooling section 506. Therefore, the reactions in the reaction mixture in the second cooling section 504 may be less susceptible to being reactions giving carbon monoxide and carbon dioxide. An advantage of having two cooling sections with OHD catalyst in the tubes for an ODH reactor may be that the tube side can be operated at lower temperature in the first cooling section 504 to reduce reactions forming carbon monoxide and carbon dioxide in the presence of higher concentrations of oxygen. For instance: (1) the reaction mixture and ODH catalyst on the tube side in the first cooling section 504 can be maintained at a temperature in a range of 300° C. to 450° C.; and (2) the reaction mixture and ODH catalyst on the tube side in the second cooling section 506 can be maintained at a temperature in a range of 350° C. to 500° C. The third cooling section 510 is disposed operationally downstream of the second cooling section 506 in the direction of the flow of the reaction mixture on the tube side.

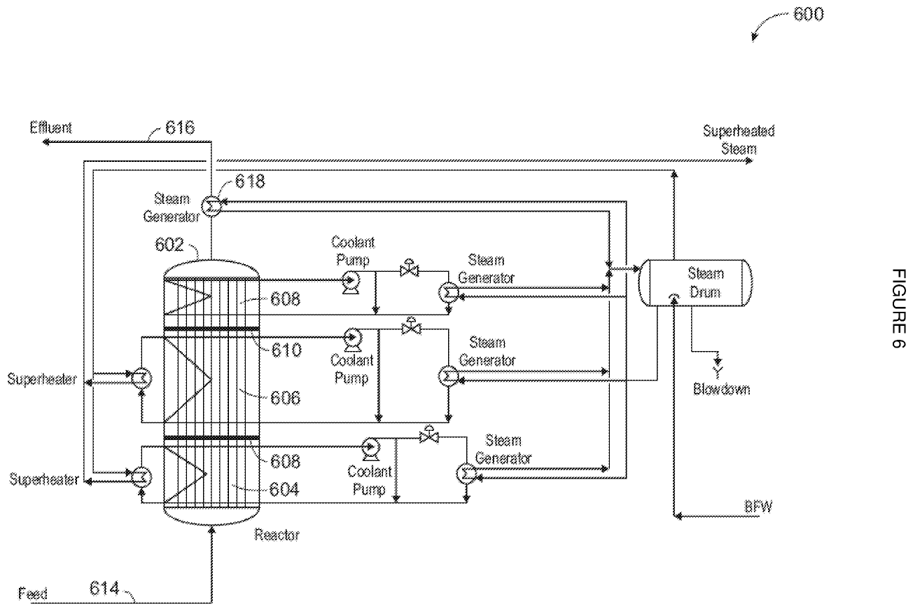

FIG. 6 is an ODH reactor system 600 that is similar to the ODH reactor system 500 of FIG. 5, except that the third cooling section of the sole ODH reactor 602 has ODH catalyst, and the ODH reactor system 600 includes an effluent heat exchanger to cool the effluent. Refer to FIGS. 3-5 for a discussion of similar or same equipment and operation. The ODH reactor system 600 may be the ODH reactor system 102 of FIG. 1. For comparison of examples of ODH reactor systems disclosed herein, an implementation of the ODH reactor system 600 provides a basis for Configuration 7. The inclusion of the third cooling section may give better control of the ODH catalytic reaction and thus could beneficially result in less generation of CO and CO₂. An increasing number of cooling sections (catalyst cooling sections) may result in better selectivity towards ethylene due to the tube side can be operated at lower temperature in upstream cooling sections to reduce reactions forming CO and CO₂ in presence of higher concentrations of oxygen. In general, the reaction-mixture temperature may increase from a cooling section to the next cooling section.

In having more cooling sections in the same ODH reactor, better performance may be realized in implementations. However, an increasing number of cooling sections can make the ODH reactor system more complex. Increased complexity may not be desirable in certain instances. While three or more cooling sections may be implemented in a single reactor, certain implementations may have a maximum of two cooling sections in the single reactor. For an ODH reactor system as a multi-reactor system having two reactors in series, an example is the first reactor has two catalyst cooling sections and one inert cooling section (salt quenching) and the second reactor in series has two catalyst cooling sections. This scenario or reactor system configuration can be applicable to any number of reactors in series (the last reactor having two catalyst cooling sections and previous stages having two catalyst-cooling sections and one salt quenching section). Of course, other configurations are applicable.

For the ODH reactor system 600, the ODH reactor 602 has a first cooling section 604, a second cooling section 606, and a third cooling section 608. The second cooling section 606 is disposed operationally downstream of the first cooling section 604 in the direction of flow of the reaction mixture. The third cooling section 608 is disposed operationally downstream of the second cooling section 606 in the direction of flow of the reaction mixture.

A barrier 610 (e.g., a metal plate with perforations for tubes) separates the first cooling section 604 and the second cooling section 606 on the shell side of the reactor 602. A barrier 612 (e.g., a metal plate with perforations for tubes) separates the second cooling section 606 and the third cooling section 608 on the shell side of the reactor 602. The reaction mixture flows on the tube side through the tubes through the three cooling sections 604, 606, 608 and its flow is unaffected by the barriers 610, 612. ODH catalyst is disposed in the tubes in the three cooling sections 604, 606, 608.

The feed 614 to the reactor 602 as introduced to the tubes (tube side) may give the initial reaction mixture as entering the first cooling section 604. The feed 614 may be analogous to the feed 104 of FIG. 1. The reaction mixture flows through the tubes. The tubes are routed through the barriers 610, 612. The barriers 610, 612 are not inside the tubes. Again, the flow of the reaction mixture is unaffected by the barriers 610, 612. The reaction mixture flowing in the tubes flows from the first cooling section 604 across the barrier 610 into the second cooling section 606. The reaction mixture flows in the tubes from the second cooling section 606 across the barrier 612 into the third cooling section 608. The reaction mixture as discharged from the tubes (tube side) of the third cooling section 608 may give the effluent 616 of the ODH reactor 602. The reaction mixture from the third cooling section 608 discharges as the effluent 616. The effluent 616 may be analogous to the effluent 108 of FIG. 1.

The cooling sections 604, 606, 608 may each give a respective isotherm temperature for the reactor 602. In particular, the cooling sections 604, 606, 608 may each give or facilitate a respective constant temperature of the reaction mixture and ODH catalyst on the tube side. For example, this temperature may be in the range of 300° C. to 450° C. in the first cooling section 604 and in the range of 350° C. to 500° C. in the cooling sections 606, 608.

As discussed (e.g., with respect to FIGS. 3-5), a coolant supply system for an ODH reactor may include a coolant pump (e.g., centrifugal pump) to receive the coolant from the shell side of the ODH reactor and to provide motive force for the circulation of coolant through the shell side. The coolant pump may be configured to give a specified capacity (circulation flow rate of the coolant) based on a specified temperature increase for the coolant through the shell side. The coolant supply system may include a coolant heat exchanger to cool at least a portion of the coolant to give a desired or specified coolant supply temperature to the shell side. The coolant supply system may include a control valve (e.g., flow control valve) to regulate the amount of the coolant in the circulation routed through the coolant heat exchanger.

In FIG. 6, a first coolant supply system circulates coolant (e.g., molten salt) via a coolant pump through the shell side in the first cooling section 604, and thus provides coolant supply to the first cooling section 604 and receives coolant return from the first cooling section 604 (to the coolant pump). A second coolant supply system circulates coolant (e.g., molten salt) via a coolant pump through the shell side in the second cooling section 606, and thus provides coolant supply to the second cooling section 606 and receives coolant return from the first cooling section 606 (to the coolant pump suction). A third coolant supply system circulates coolant (e.g., molten salt) via a coolant pump through the shell side in the third cooling section 608, and thus provides coolant supply to the third cooling section 608 and receives coolant return from the third cooling section 608 (to the coolant pump inlet).

The respective coolant heat exchanger in each of the first coolant supply system, the second coolant supply system, and the third coolant supply system may each receive water (e.g., BFW) from the steam drum and heat the water (with coolant as the heating medium) to vaporize the water to give steam (e.g., HP/VHP steam) to the steam drum. An effluent heat exchanger 618 (disposed along the effluent discharge conduit from the reactor 602) may receive water from the steam drum and heat the water (with effluent 616 as the heating medium) to vaporize the water to give steam (e.g., HP/VHP steam) to the steam drum. Thus, the effluent heat exchanger 618 may beneficially cool (remove heat from) the effluent 616. In implementations, the effluent heat exchanger 618 may cool the effluent 616, for example, to a temperature in a range of 150° C. to 350° C. (or in the range of 200° C. to 300° C.).

As discussed, steam may discharge from the steam drum for distribution to users. As also discussed, the steam may be superheated in a heat exchanger (e.g., as in 332 of FIG. 3) associated with a cooling section prior to distribution of the steam to users. In FIG. 6, there are two such heat exchangers associated with the first cooling section 604 and the second cooling section 606, respectively. A third such heat exchanger (superheater) (not shown) may also be associated with the third cooling section 608.

The heating medium for the two superheater heat exchangers is the coolant flowing through the shell side of the first cooling section 604 and the second cooling section 606, respectively. These two superheater heat exchangers may each be configured as discussed with respect to corresponding superheater heat exchangers in FIGS. 3-5. As mentioned, a third superheated heat exchanger coupled with the third cooling section 608 may be similarly configured. In general, the number of such superheater heat exchangers for the reactor system 600 may be one associated with one cooling section, two associated with two cooling sections, respectively, or three associated with the three cooling sections, respectively. The coolant (e.g., molten salt) is utilized to superheat the steam.

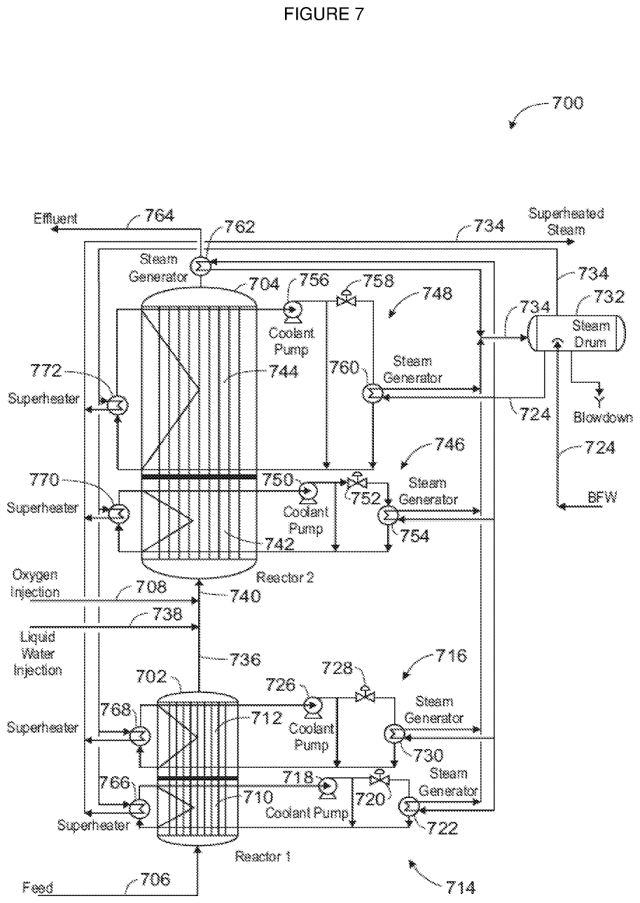

FIG. 7 is an ODH reactor system 700 that is similar to the ODH reactor systems of FIGS. 3-6, except that the ODH reactor system 700 is not a single-reactor system. Instead, the ODH reactor system 700 is a multi-reactor system having two OHD reactors 704, 706 disposed operationally in series. Refer to FIGS. 3-6 for a discussion of similar or same equipment and operation. The ODH reactor system 700 may be the ODH reactor system 102 of FIG. 1. For comparison of examples of ODH reactor systems disclosed herein, an implementation of the ODH reactor system 700 provides a basis for Configuration 8.

The first ODH reactor 702 and the second ODH reactor 704 are each a multi-tubular fixed bed reactor have two cooling sections that employ coolant, such as molten salt, on the reactor shell side to cool the reaction mixture and ODH catalyst on the tube side. The cooling sections 710, 712, 742, 744 may each give or facilitate a respective isotherm temperature (constant temperature) of the reaction mixture and ODH catalyst on the tube side. For example, this tube-side temperature may be maintained in the range of 300° C. to 450° C. in the first cooling sections 710, 742 and in the range of 350° C. to 500° C. in the second cooling sections 712, 744.

The two cooling sections in each reactor 702, 704 are separated by a respective barrier on the shell side (sealing the shell side between cooling sections). The reaction mixture flows through the tubes (on the tube side) through the each barrier. The tubes (tube side) in the four cooling sections have ODH catalyst.

The first ODH reactor 702 is disposed operationally upstream of the second ODH reactor 704. The first ODH reactor 702 receives feed 706 that may be analogous to the feed 104 of FIG. 1. The feed 706 may beneficially have less oxygen gas (and therefore less dilution steam) because oxygen 708 gas is injected between the reactors 702, 704. That can be an advantage of a multi-reactor system compared to a single-reactor system. The feed 706 may include an adequate amount of oxygen (or more) for the OHD reaction in the first ODH reactor 702.

The feed 706 (first-reactor feed) introduced to the first ODH reactor 702 gives the initial reaction mixture flowing through the tube side. The reaction mixture (on the tube side) flows through the first cooling section 710 of the first reactor 702 and through the second cooling section 712 of the first reactor 702. The second cooling section 712 is disposed operationally downstream of the first cooling section 710.

A first-coolant supply system 714 provides a first coolant as coolant supply to the reactor shell side in the first cooling section 710, and receives the first coolant as coolant return from the reactor shell side in the first cooling section 710. A second-coolant supply system 716 provides a second coolant as coolant supply to the reactor shell side in the second cooling section 712, and receives the second coolant as coolant return from the reactor shell side in the second cooling section 712. Both the first coolant and the second coolant may be, for example, molten salt.

The first-coolant supply system 714 may have a first-coolant pump 718, a first-coolant control valve 720, and a first-coolant heat exchanger 722 that cools first coolant with water 724 as cooling medium. The first-coolant pump 718 (e.g., centrifugal pump) as a first-coolant circulation pump may be configured for a flow rate (circulation rate) of the first coolant to give a specified temperature increase of the first coolant through the shell side in the first cooling section 710. The first-coolant control valve 720 (e.g., flow control valve) may regulate the amount (flow rate) of first coolant routed through the first-coolant heat exchanger 722. The first-coolant heat exchanger 722 (e.g., shell-and-tube heat exchanger) may remove the heat of reaction received by the first coolant in the reactor shell side to give the desired supply temperature of the first coolant to the reactor shell side.

The second-coolant supply system 716 may have a second-coolant pump 726, a second-coolant control valve 728, and a second-coolant heat exchanger 730 that cools the second coolant with water 724 as cooling medium. The second-coolant pump 726 (e.g., centrifugal pump) as a second-coolant circulation pump may be configured for a flow rate (circulation rate) of the second coolant to give a specified temperature increase of the second coolant through the shell side in the first cooling section 710. The second-coolant control valve 728 (e.g., flow control valve) may regulate the amount (flow rate) of second coolant routed through the second-coolant heat exchanger 730. The second-coolant heat exchanger 730 (e.g., shell-and-tube heat exchanger) may remove the heat of reaction received by the second coolant in the reactor shell side to give the desired supply temperature of the second coolant to the reactor shell side.

The first-coolant heat exchanger 722 and the second-coolant heat exchanger 730 may each receive water 724 from the steam drum 732 vessel, and vaporize the water 724 into steam 734 sent to the steam drum 732. The water 724 is provided to the steam drum 732 from a source (not shown). For the water 724 as boiler feedwater (BFW), the source may be a BFW pump.

The reaction mixture discharges from the first ODH reactor 702 from the tube side as effluent 736 of the first ODH reactor 702. This effluent 736 may include ethylene, acetic acid (vapor), CO, CO₂, water (vapor), unreacted ethane, and residual oxygen. The effluent 736 may discharge through an effluent discharge conduit from the first ODH reactor 702. This effluent discharge conduit may convey the effluent 736 to the feed inlet of the second ODH reactor 702. Therefore, the effluent discharge conduit from the first ODH reactor 702 may serve as a feed conduit to the second ODH reactor 702.

Liquid water 738 is added to (injected into) the effluent 736 as a water quenching of the effluent 736 to cool the effluent 736. In particular, vaporization of the added liquid water 738 in the effluent 738 consumes heat in the effluent 736 to cool the effluent 736. In other words, sensible heat of the effluent 736 may provide the latent heat of vaporization to vaporize the injected liquid water 738, thereby cooling the effluent 736. In implementations, a liquid-water supply conduit conveys the liquid water 738 to the effluent discharge conduit conveying the effluent 736. A pipe tee may couple the liquid-water supply conduit with the effluent discharge conduit for addition (injection) of the liquid water 738 into the effluent 736. The water quench via the injected liquid water 738 may cool the effluent 736, for example, to a temperature in a range of 150° C. to 350° C. (or in the range of 200° C. to 300° C.). After the water quench, O₂ may be injected into the effluent 736. A purpose of cooling the effluent 736 (whether by the depicted water quench or by other cooling techniques) may be to avoid autoignition of hydrocarbons and acetic acid (which may be at or near their autoignition temperature) with the addition of the O₂, and avoid associated undesirable reactions (e.g., burning and generating CO and CO₂).

Oxygen 708 gas may be added to (injected into) the effluent 736. The amount of the oxygen 708 added to the effluent 736 provides oxygen for the ODH reaction in the second ODH reactor 704. (The amount of oxygen gas included in the upstream feed 706 to the first ODH reactor 702 may be adequate for the ODH reaction in the reaction mixture in the first ODH reactor 702.) In implementations, an oxygen supply conduit conveys the oxygen 708 gas to the effluent discharge conduit conveying the effluent 736. A pipe tee may couple the oxygen supply conduit with the effluent discharge conduit for addition (injection) of the oxygen 708 into the effluent 736.

In the illustrated embodiment, the feed 740 provided to the second ODH reactor 704 includes the first-reactor effluent 736, the added liquid water 738 as vaporized, and the added oxygen 708 gas. The effluent discharge conduit from the first ODH reactor 702 may be or couple to the feed conduit that couples to a feed inlet (e.g., vessel inlet nozzle) of the second ODH reactor 704.

The feed 740 (second-reactor feed) introduced to the second ODH reactor 704 gives the initial reaction mixture of the second ODH reactor 704 flowing through the tube side. The reaction mixture (on the tube side) flows through the first cooling section 742 of the second ODH reactor 704 and through the second cooling section 744 of the second ODH reactor 702. The second cooling section 744 is disposed operationally downstream of the first cooling section 742. In the context of the ODH reactor system 100, the first cooling section 742 can be labeled as a third cooling section, and the second cooling section 744 labeled as a fourth cooling section of the four cooling sections.

A third-coolant supply system 746 provides a third coolant as coolant supply to the reactor 704 shell side in the first cooling section 742, and receives the first coolant as coolant return from the reactor 704 shell side in the first cooling section 710. A fourth-coolant supply system 748 provides a fourth coolant as coolant supply to the reactor 704 shell side in the second cooling section 744, and receives the fourth coolant as coolant return from the reactor 704 shell side in the second cooling section 744. Both the third coolant and the fourth coolant may each be, for example, molten salt.

The third-coolant supply system 746 may have a third-coolant pump 750, a third-coolant control valve 752, and a third-coolant heat exchanger 754. The third-coolant heat exchanger 754 cools third coolant with water 724 as cooling medium. The third-coolant pump 750 (e.g., centrifugal pump) as a third-coolant circulation pump may be configured for a flow rate (circulation rate) of the third coolant to give a specified temperature increase of the third coolant through the shell side in the first cooling section 742. The third-coolant control valve 752 (e.g., flow control valve) may regulate the amount (flow rate) of third coolant routed through the third-coolant heat exchanger 754. The third-coolant heat exchanger 754 (e.g., shell-and-tube heat exchanger) may remove the heat of reaction received by the third coolant in the reactor 704 shell side to give the desired supply temperature of the third coolant to the reactor 704 shell side.

The fourth-coolant supply system 748 may have a fourth-coolant pump 756, a fourth-coolant control valve 758, and a fourth-coolant heat exchanger 760 that cools the fourth coolant with water 724 as cooling medium. The fourth-coolant pump 756 (e.g., centrifugal pump) as a fourth-coolant circulation pump may be configured for a flow rate (circulation rate) of the fourth coolant to give a specified temperature increase of the fourth coolant through the shell side of the second cooling section 744. The fourth-coolant control valve 758 (e.g., flow control valve) may regulate the amount (flow rate) of fourth coolant routed through the fourth-coolant heat exchanger 760. The fourth-coolant heat exchanger 760 (e.g., shell-and-tube heat exchanger) may remove the heat of reaction received by the fourth coolant in the reactor 704 shell side to give the desired supply temperature of the second coolant to the reactor 704 shell side.

The third-coolant heat exchanger 754 and the fourth-coolant heat exchanger 760 may each receive water 724 from the steam drum 732 vessel, and vaporize the water 724 (with coolant as heating medium) into steam 734 sent to the steam drum 732. In addition, an effluent heat exchanger 762 may heat and vaporize water 724 (with effluent 764 from the second ODH reactor 704 as heating medium) into steam 734 sent to the steam drum 732.

The reaction mixture discharges from the second ODH reactor 704 from the tube side as effluent 762. The effluent 762 may be a product effluent analogous to the effluent 108 of FIG. 1. The effluent 762 may include ethylene, acetic acid (vapor), CO, CO₂, water (vapor or steam), and unreacted ethane. The effluent 762 may discharge through an effluent discharge conduit from the second ODH reactor 704. The effluent discharge conduit may be coupled to an effluent outlet nozzle on the second ODH reactor 704 to receive the effluent 762. The aforementioned effluent heat exchanger 762 may disposed along the effluent discharge conduit conveying the effluent 764. The effluent heat exchanger 762 may cool the effluent 762 with water 724 as cooling medium. As mentioned, the effluent heat exchanger 762 may vaporize the water 724 with the effluent 762 as heating medium to generate steam 734.

In implementations of the ODH reactor system 700, the steam 734 generated at the heat exchangers 722, 730, 754, 760, and 762 may be HP/VHP steam. Therefore, the steam drum 732 vessel may operate at HP/VHP (e.g., pressure in the range of 500 psig to 1700 psig). Thus, the steam 734 that discharges from the steam drum 732 for distribution may be HP/VHP steam that is generally saturated steam. Therefore, the steam 734 as discharged from the steam drum 732 may be, for example, at a temperature (saturation temperature) in a range of 240° C. to 330° C.

The ODH reactor system 700 may heat the steam 734 to above saturation temperature to give the steam 734 as superheated steam (HP/VHP) for distribution. In particular, the superheater heat exchangers 766, 768, 770, and 772 may receive the steam 734 from the steam drum 732 and heat the steam 734 (with reactor shell-side coolant as heating medium) to superheat the steam 734. While the superheater heat exchangers are depicted as receiving the seam 734 in parallel, they could be arranged operationally in series. As depicted, each heat exchanger 766, 768, 770, 772 may be associated with a respective cooling section 710, 712, 742, 744. These four superheater heat exchangers 766, 768, 770, 772 may each be configured as discussed with respect to corresponding superheater heat exchangers in FIGS. 3-6.

FIG. 8 is an ODH reactor system 800 that is the same or similar as the ODH reactor system 700 of FIG. 7, except that the first ODH reactor 802 has a third cooling section 804 (no catalyst) in lieu of a water quench between the ODH reactors in FIG. 7. For the coolant as molten salt, the third cooling section 804 can be characterized as a salt quench. An advantage of the third cooling section 804 can be that the heat removed from the reaction mixture can be recovered to generate steam via a coolant heat exchanger. In contrast, for a water quench, the removed heat may be lost in the vaporization of the water. Moreover, as discussed, the cooling of the effluent before inter-stage O₂ addition may be beneficial to lower temperature of effluent components (e.g., acetic acid) to below their autoignition temperature (if above their autoignition temperature) and thus prevent autoignition (and associated combustion of materials such as ethane, ethylene, acetic acid, CO, etc.).

For the ODH reactor system 800 generally, refer to FIGS. 3-7 for a discussion of similar or same equipment and operation. The ODH reactor system 800 may be the ODH reactor system 102 of FIG. 1. For comparison of examples of ODH reactor systems disclosed herein, an implementation of the ODH reactor system 800 provides a basis for Configuration 9.

The third cooling section 804 (of the first ODH reactor 802) may be a non-catalyst cooling section in that there may be no catalyst in the tubes in the third cooling section 804 (but may have inert particles disposed therein). This third cooling section 804 and associated fifth-coolant supply system (circulating fifth coolant through the reactor shell side in the third cooling section 804) provides for a respective isotherm in the sense that the reaction mixture flowing through the tubes in the third cooling section 804 is maintained at a specified temperature, for example, in a range of 150° C. to 350° C. (or in the range of 200° C. to 300° C.).

The fifth-coolant heat exchanger (for the fifth coolant, e.g., molten salt) in the fifth-coolant supply system associated with the third cooling section 804 preheats water (e.g., BFW) for vaporization of the water in the steam drum. The water may be provided from a pump and generally at the operating pressure (e.g., HP/VHP) of the steam drum. The water may flow from the pump (e.g., BFW pump) through the fifth-coolant heat exchanger to the steam drum.

Again, a liquid level of the water may be maintained in the steam drum. Steam may discharge from the steam drum for distribution to users. The steam after discharge from the steam drum may be superheated in superheater heat exchangers, for example, as discussed with respect to FIG. 7, prior to distribution to users.

The first ODH reactor 802 receives feed 806 that may be same or similar as the feed 706 of FIG. 7. The feed 806 enters the tube side of the first ODH reactor 802 to give the flowing reaction mixture in the tubes. The reaction mixture flows through the first and second cooling sections of the first reactor 802, as in the ODH reactor system 700 of FIG. 7. However, in the reactor system 800, the reaction mixture additionally flows through the tube side of the third cooling section 804 of the first ODH reactor 802. Again, the reactor tubes in the third cooling section 804 may not have catalyst.

The reaction mixture discharges from the first ODH reactor 802 from the tube side as effluent 808 (first-reactor effluent) for feed to the tube side of the second ODH reactor 810. The effluent 808 may be the similar or same compositionally as the effluent 736 of FIG. 7, but is typically at a lower temperature than the effluent 736 as discharged because of the presence of the third cooling section 804 of the first ODH reactor 802 in FIG. 8. Oxygen gas may be injected into effluent 808, as with the ODH reactor system 700 of FIG. 7. However, injecting liquid water for a water quench into the effluent as in FIG. 7 may be avoided because the third cooling section 804 cools the reaction mixture that discharges as the effluent 808. The effluent 808 plus the injected oxygen enters as feed to the second ODH reactor 810 to give a reaction mixture flowing through the tube side. This reaction mixture flows in the tubes through the two cooling sections and discharges as effluent 812 through an effluent heat exchanger, as in FIG. 7. The effluent 812 may be the same or similar as the effluent 764 of FIG. 7. The effluent 812 may be analogous to the effluent 108 of FIG. 1. As in FIG. 7, the effluent heat exchanger and four coolant heat exchangers generate steam from water received from the steam drum and discharge the steam to the steam drum. Four superheater heat exchangers superheat the steam discharged from the steam drum for distribution.

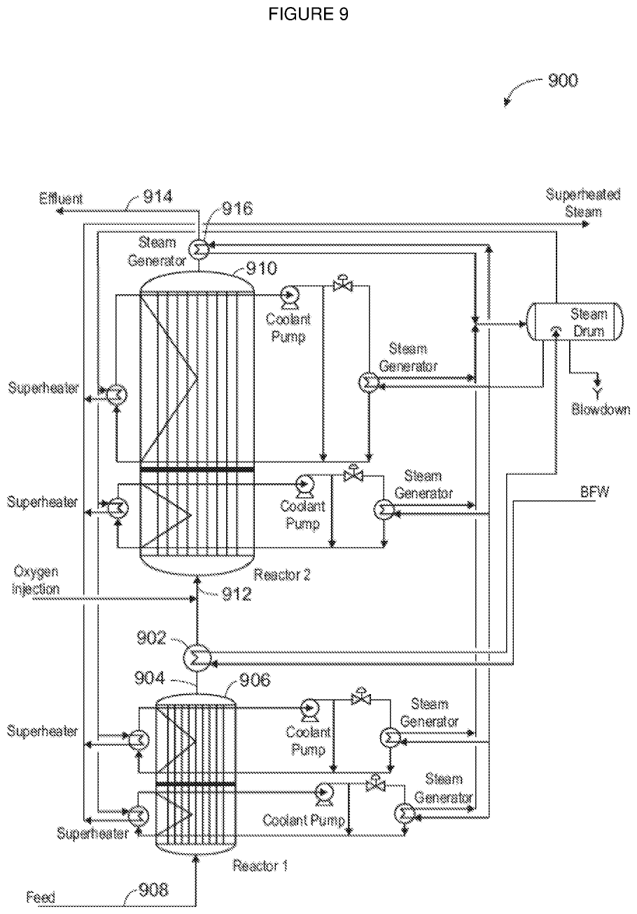

FIG. 9 is an ODH reactor system 900 that is the same or similar as the ODH reactor system 700 of FIG. 7, except that an effluent heat exchanger 902 (instead of an injected-water quench) cools the effluent 904 discharged from the first ODH reactor 906. An advantage of the effluent heat exchanger 902 can be that the heat removed from the effluent 904 can be recovered to preheat water (e.g., BFW) for steam generation. In contrast, for the injected-water quench, the removed heat may be lost in the vaporization of the injected water in the effluent 904.

For the ODH reactor system 900 generally, refer to FIGS. 3-8 for a discussion of similar or same equipment and operation. The ODH reactor system 900 may be the ODH reactor system 102 of FIG. 1. For comparison of examples of ODH reactor systems disclosed herein, an implementation of the ODH reactor system 900 provides a basis for Configuration 10.

The effluent heat exchanger 902 (e.g., a shell-and-tube heat exchanger) may cool the effluent 904 with water (e.g., BFW) to a specified temperature, for example, in a range of 150° C. to 350° C. (or in the range of 200° C. to 300° C.). The effluent heat exchanger 902 may receive the water (e.g., from a BFW pump) and heat the water (in the cooling of the effluent) that pre-heats the water for vaporization of the water in the steam drum. The water may be provided to the effluent heat exchanger 902 generally at the operating pressure (e.g., HP/VHP) of the steam drum. The water may flow from the pump (e.g., BFW pump) through the effluent heat exchanger 902 to the steam drum.

As discussed, a liquid level of the water may be maintained in the steam drum. Steam may discharge from the steam drum for distribution to users. The steam after discharge from the steam drum may be superheated in superheater heat exchangers prior to distribution to users, as discussed with respect to FIG. 7.

The first ODH reactor 906 receives feed 908 that may be same or similar as the feed 706 of FIG. 7. The feed 908 enters the tube side of the first ODH reactor 906 to give the flowing reaction mixture in the tubes. The reaction mixture flows through the first and second cooling sections of the first reactor 906 and discharges as the effluent 904 (first-reactor effluent), as in the ODH reactor system 700 of FIG. 7. However, liquid water is not injected into the effluent 904, as in FIG. 7. Instead, the effluent heat exchanger 902 (not in FIG. 7 for the first-reactor effluent) cools the effluent 904. Again, it may be desirable to cool the effluent 904, for example, to cool any components approaching or above their autoignition temperature to well below their autoignition temperature.

The reaction mixture discharges from the first ODH reactor 902 from the tube side as the effluent 902 (first-reactor effluent) for feed to the tube side of the second ODH reactor 910. The effluent 904 may have the same or similar composition as the effluent 736 of FIG. 7. Oxygen gas may be injected into effluent 904, as with the ODH reactor system 700 of FIG. 7, to give the feed 912 to the second OHD reactor 910. However, again, injecting liquid water for a water quench into the effluent as in FIG. 7 may be avoided because the effluent heat exchanger 902 cools the effluent 904. The effluent 904 plus the injected oxygen enters as the feed 912 to the second ODH reactor 910 to give a reaction mixture flowing through the tube side. This reaction mixture flows in the tubes through the two cooling sections of the second OHD reactor 910 and discharges from the tube side from the second ODH reactor 910 as effluent 914 (second-reactor effluent) through an effluent heat exchanger 916, as in FIG. 7. The effluent 914 may be the same or similar as the effluent 764 of FIG. 7. The effluent 914 may be analogous to the effluent 108 of FIG. 1. As in FIG. 7, the effluent heat exchanger 916 and four coolant heat exchangers generate steam from water received from the steam drum and discharge the steam to the steam drum. Four superheater heat exchangers (as previously discussed) superheat the steam discharged from the steam drum for distribution.

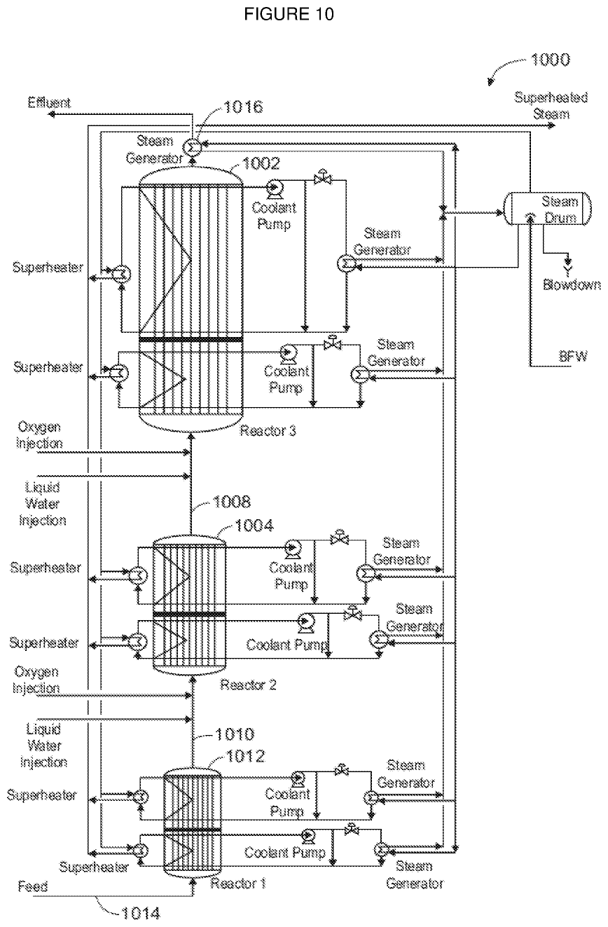

FIG. 10 is an ODH reactor system 1000 that is the same or similar as the ODH reactor system 700 of FIG. 7, except that the ODH reactor system 1000 has a third ODH reactor 1002 disposed operationally in series downstream of the second ODH reactor 1004. Thus, the ODH reactor system 1000 has three ODH reactors in series. In present embodiments of ODH reactors systems, the number “n” of ODH reactors in series can be more than three. The greater the number “n” of ODH reactors in series can be advantageous from performance and energy considerations of the ODH plant. However, the greater the number “n” of ODH reactors in series can add operational complexity and capital cost of ODH reactor system.

In the ODH reactor system 1000, the effluent 1006 (third-reactor effluent) that discharges from the third ODH reactor 1002 is the product effluent analogous to the effluent 108 of FIG. 1. The effluent 1008 that discharges from the second ODH reactor 1004 is feed to the third ODH reactor 1002. Both liquid water (for water quench cooling) and oxygen may be injected into the effluent 1008 (second-reactor effluent), as with the effluent 1010 (first-reactor effluent) that discharges from the first ODH reactor 1012 in the series of the three ODH reactors of the ODH reactor system 1000.

A fifth-coolant supply system circulates (via coolant pump) fifth coolant through the shell side in the first cooling section of the third ODH reactor 1002. The fifth-coolant supply system removes heat from the circulating fifth coolant via a coolant heat exchanger (with water as cooling medium) that also generates steam from the water (e.g., BFW). The coolant heat exchanger receives the water from the steam drum and discharges the generated steam to the steam drum.

As with other coolant supply systems previously discussed, a sixth-coolant supply system circulates (via coolant pump) sixth coolant through the shell side in the second cooling section of the third ODH reactor 1002. The sixth-coolant supply system removes heat from the circulating sixth coolant via a coolant heat exchanger (with water as cooling medium) that also generates steam from the water (e.g., BFW). The coolant heat exchanger receives the water from the steam drum and discharges the generated steam to the steam drum. The sixth coolant and the fifth coolant may each be, for example, molten salt.

For the ODH reactor system 1000 generally, refer to FIGS. 3-9 for a discussion of similar or same equipment and operation. The ODH reactor system 1000 may be the ODH reactor system 102 of FIG. 1. For comparison of examples of ODH reactor systems disclosed herein, an implementation of the ODH reactor system 1000 provides a basis for Configuration 11.

As with the ODH reactor system 700 of FIG. 7, the first ODH reactor 1012 receives feed 1014 that is analogous to the feed 104 of FIG. 1. Advantageously, the feed 1014 may have less oxygen gas (and therefore less dilution steam) than with a single-reactor system because oxygen gas is injected between the ODH reactors in the series in a multi-reactor system. That can be a benefit of a multi-reactor system compared to a single-reactor system.

The first ODH reactor 1012, the second ODH reactor 1004, and the third ODH reactor 1002 are each a multi-tubular fixed bed reactor have two cooling sections that employ coolant, such as molten salt, on the reactor shell side to cool the reaction mixture and ODH catalyst on the tube side. The six cooling sections may each give or facilitate a respective isotherm temperature (constant temperature) of the reaction mixture and ODH catalyst on the tube side. For example, this tube-side temperature may be maintained in the range of 300° C. to 450° C. in the first cooling sections of each reactor and in the range of 350° C. to 500° C. in the second cooling sections of each reactor. The two cooling sections in each ODH reactor are separated by a respective barrier on the shell side (sealing the shell side between cooling sections). The reaction mixture flows through the tubes (on the tube side) across each barrier. The barriers are not inside the tubes. The tubes (tube side) in the six cooling sections have ODH catalyst.

The feed 1014 (first-reactor feed) introduced to the first ODH reactor 1012 gives the initial reaction mixture flowing through the tube side. The reaction mixture (on the tube side) flows through the first cooling section of the first ODH reactor 1012 and through the second cooling section of the first ODH reactor 1012 disposed operationally downstream of the first cooling section of the first ODH reactor 1012. The reaction mixture discharges from the first ODH reactor 1012 from the tube side as the effluent 1010 of the first ODH reactor 1012. This effluent 1010 may include ethylene, acetic acid (vapor), CO, CO₂, water (vapor), unreacted ethane, and residual oxygen. The effluent 1010 may discharge through an effluent discharge conduit from the first ODH reactor 1012. This effluent discharge conduit may convey the effluent 1010 to the feed inlet of the second ODH reactor 1004. Therefore, the effluent discharge conduit from the first ODH reactor 1012 may serve as a feed conduit to the second ODH reactor 1004.

Liquid water may be added to (injected into) the effluent 1010 as a water quenching of the effluent 1010 to cool the effluent 1010. In particular, vaporization of the added liquid water in the effluent 1010 consumes heat in the effluent 1010 to cool the effluent 1010. In other words, sensible heat of the effluent 1010 may provide the latent heat of vaporization to vaporize the injected liquid water, thereby cooling the effluent 1010. This water quench via the injected liquid water may cool the effluent 1010, for example, to a temperature in a range of 150° C. to 350° C. (or in the range of 200° C. to 300° C.). Further, oxygen gas may be added to (injected into) the effluent 1010. The oxygen added to the effluent 1010 provides oxygen for the ODH reaction in the tubes (via ODH catalyst) of the second ODH reactor 1004.

Thus, the feed provided to the second ODH reactor 1004 includes the first-reactor effluent 1010, the added liquid water as vaporized, and the added oxygen gas. The effluent discharge conduit from the first ODH reactor 1004 may be or couple to the second-reactor feed conduit that couples to a feed inlet (e.g., vessel inlet nozzle) of the second ODH reactor 1004. This feed (second-reactor feed) introduced to the second ODH reactor 1004 gives the initial reaction mixture of the second ODH reactor 1004 flowing through the tube side. The reaction mixture (on the tube side) flows through the first cooling section of the second ODH reactor 1004 and through the second cooling section of the second ODH reactor 1004. The second cooling section of the second ODH reactor 1004 is disposed operationally downstream of the first cooling section of the second ODH reactor 1004. In the context of the ODH reactor system, the first cooling section of the second ODH reactor 1004 can be labeled as a third cooling section, and the second cooling section of the second ODH reactor 1004 can labeled as a fourth cooling section of the six cooling sections among the three ODH reactors.

The reaction mixture discharges from the second ODH reactor 1004 from the tube side as the effluent 1008 for feed to the third ODH reactor 1002. The effluent 1008 may include ethylene, acetic acid (vapor), CO, CO₂, water (vapor or steam), and unreacted ethane. The effluent 1008 may discharge through an effluent discharge conduit from the second ODH reactor 1004. The effluent discharge conduit may be coupled to an effluent outlet nozzle on the second ODH reactor 1004 to receive the effluent 1008. This effluent discharge conduit may convey the effluent 1008 to the feed inlet of the third ODH reactor 1002. Therefore, the effluent discharge conduit from the second ODH reactor 1004 may serve as a feed conduit to the third ODH reactor 1002.

As with between the first reactor 1012 and second reactor 1004, liquid water and oxygen may be injected between the second reactor 1004 and the third reactor 1002. Liquid water may be added to (injected into) the second-reactor effluent 1008 as a water quenching of the effluent 1008 via vaporization of the injected water to cool the effluent 1008. This water quench via the injected liquid water may cool the effluent 1008, for example, to a temperature in a range of 150° C. to 350° C. (or in the range of 200° C. to 300° C.). Further, oxygen gas may be added to (injected into) the second-reactor effluent 1008. The oxygen added to the effluent 1008 provides oxygen for the ODH reaction in the tubes (via ODH catalyst) of the third ODH reactor 1002.

Therefore, the feed provided to the third ODH reactor 1002 includes the second-reactor effluent 1008, the added liquid water as vaporized, and the added oxygen gas. The effluent discharge conduit from the second ODH reactor 1004 may be or couple to the third-reactor feed conduit that couples to a feed inlet (e.g., vessel inlet nozzle) of the third ODH reactor 1002. This feed (third-reactor feed) introduced to the third ODH reactor 1002 gives the initial reaction mixture of the third ODH reactor 1002 flowing through the tube side. The reaction mixture (on the tube side) flows through the first cooling section of the third ODH reactor 1002 and through the second cooling section of the third ODH reactor 1002.

The second cooling section of the third ODH reactor 1002 is disposed operationally downstream of the first cooling section of the third ODH reactor 1002. In the context of the ODH reactor system, the first cooling section of the third ODH reactor 1002 can be labeled as a fifth cooling section, and the second cooling section of the third ODH reactor 1002 can labeled as a sixth cooling section of the six cooling sections among the three ODH reactors.

The reaction mixture discharges from the third ODH reactor 1002 from the tube side as the effluent 1006 (third-reactor effluent). The effluent 1006 may be a product effluent analogous to the effluent 108 of FIG. 1. The effluent 1006 may include ethylene, acetic acid (vapor), CO, CO₂, water (vapor or steam), and unreacted ethane. The effluent 1006 may discharge through an effluent discharge conduit from the third ODH reactor 1002. The effluent discharge conduit may be coupled to an effluent outlet nozzle on the third ODH reactor 1002 to receive the effluent 1006. An effluent heat exchanger 1016 may disposed along the effluent discharge conduit conveying the effluent 1006. The effluent heat exchanger 1016 may cool the effluent 1006 with water (e.g., BFW) from the steam drum as cooling medium. The effluent heat exchanger 1016 may vaporize the water with the effluent 1006 as heating medium to generate steam sent to the steam drum.

In implementations of the ODH reactor system 1000, the steam generated at the six coolant heat exchangers and the effluent heat exchanger 1016 may be HP/VHP steam. Therefore, the steam drum vessel may operate at HP/VHP (e.g., pressure in the range of 500 psig to 1700 psig). Thus, the steam that discharges from the steam drum for distribution may be HP/VHP steam that is generally saturated steam. Therefore, the steam as discharged from the steam drum may be, for example, at a temperature (saturation temperature) in a range of 240° C. to 330° C.

The ODH reactor system 1000 may heat the steam discharged from the steam drum to above saturation temperature to give the steam as superheated steam (HP/VHP) for distribution. In particular, the six superheater heat exchangers may receive (e.g., in parallel) the steam from the steam drum and heat the steam (with reactor shell-side coolant as heating medium) to superheat the steam. As depicted, each of the six superheater heat exchangers may be associated with a respective cooling section. These six superheater heat exchangers may each be configured as discussed with respect to corresponding superheater heat exchangers in FIGS. 3-9. The number of superheater heat exchangers could be less than six, such as in the range of 1 to 6, and may be in parallel as depicted or could be in series with respect to the flowing steam being superheated. Such alternate configurations concerning superheater exchangers in an ODH reactor system may be applicable to ODH reactor systems depicted herein with more than one superheater.

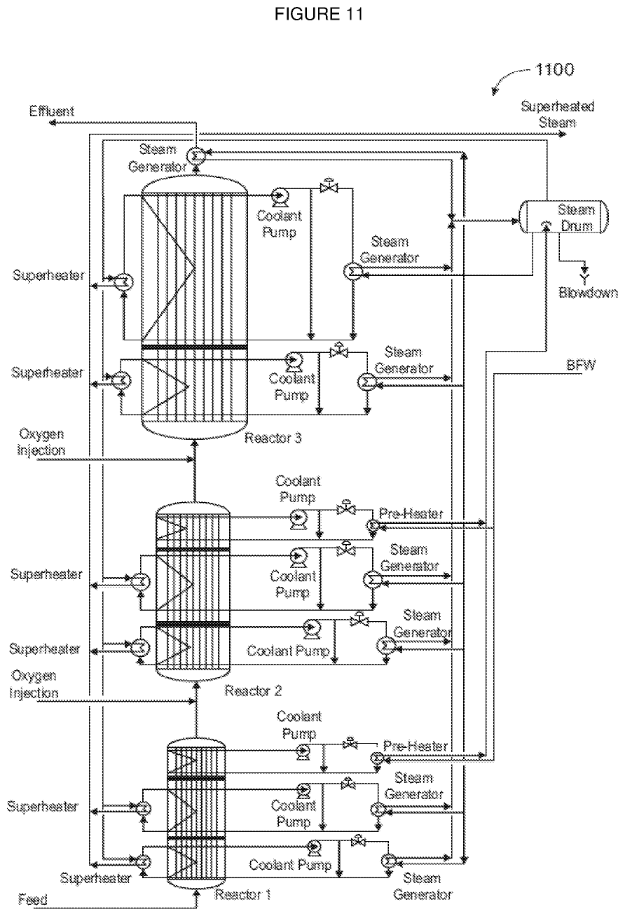

FIG. 11 is an ODH reactor system 1100 that is the same or similar as the ODH reactor system 1000 of FIG. 10, except instead of a water quench between the ODH reactors, a coolant (e.g., molten salt) quench is performed in each of the first ODH reactor and the second ODH reactor. In particular, the first ODH reactor system and the second ODH reactor each have third cooling section with no catalyst. See, for example, FIGS. 5 and 8 for a discussion of a third cooling section having no catalyst. The third cooling section (non-catalyst cooling) section in each of the first and second ODH reactors removes heat from the reaction mixture on the tube side via circulating coolant on the shell side. The third cooling section may cool the reaction mixture, for example, to a temperature in a range of 150° C. to 300° C. (or in the range of 200° C. to 300° C.). For the coolant as molten salt, the third cooling sections can be characterized as performing a salt quench. An advantage of the third cooling sections can be that the heat removed from the reaction mixture can be recovered to heat water (e.g., pre-heat BFW) via a coolant heat exchanger for steam generation. In contrast, for a water quench as in FIGS. 7 and 10, the removed heat may be lost in the vaporization of the water.

For the ODH reactor system 1100 generally, refer to FIGS. 3-10 for a discussion of similar or same equipment and operation. The ODH reactor system 1100 may be the ODH reactor system 102 of FIG. 1. For comparison of examples of ODH reactor systems disclosed herein, an implementation of the ODH reactor system 1100 provides a basis for Configuration 12.

The third cooling section (a non-catalyst cooling section) of the first ODH reactor is operationally downstream of the second cooling section of the first ODH reactor. A seventh-coolant supply system circulates (via a coolant circulation pump) seventh coolant through the shell side in the third cooling section of the first ODH reactor. A seventh-coolant heat exchanger (with water as cooling medium) removes heat from the circulating seventh coolant and thus heats (e.g., pre-heats) the water for vaporization of the water (e.g., BFW) into steam (e.g., HP/VHP) in the steam drum. The seventh-coolant heat exchanger may be configured to vaporize the water, partially vaporize the water, or heat the water without vaporization in the heat exchanger. The illustrated embodiment is heat (pre-heat) the water without significant vaporization in the heat exchanger. The seventh-coolant heat exchanger receives the water from a pump, such as a BFW pump.

The third cooling section (a non-catalyst cooling section) of the second ODH reactor is operationally downstream of the second cooling section of the second ODH reactor. An eighth-coolant supply system circulates (via a coolant circulation pump) eight coolant through the shell side in the third cooling section of the second ODH reactor. An eighth-coolant heat exchanger (with water as cooling medium) removes heat from the circulating eighth coolant and thus heats the water for vaporization of the water (e.g., BFW) into steam (e.g., HP/VHP) in the steam drum. The eighth-coolant heat exchanger may be configured to vaporize the water, partially vaporize the water, or heat the water without vaporization in the heat exchanger. The illustrated embodiment is heat (pre-heat) the water without significant vaporization in the heat exchanger. The eighth-coolant heat exchanger receives the water from a pump, such as a BFW pump.

The seventh coolant and the eighth coolant may each be, for example, molten salt. In the context of the ODH reactor system 1100, the third cooling section of the first ODH reactor can be labeled as a seventh cooling section, and the third cooling section of the second ODH reactor can labeled as an eighth cooling section of the eight cooling sections among the three ODH reactors.

Again, a liquid level of the water may be maintained in the steam drum. Steam may discharge from the steam drum for distribution to users. The steam after discharge from the steam drum may be superheated in superheater heat exchangers (previously discussed) prior to distribution to users.

The first ODH reactor receives feed that may be same or similar as the feed 1014 of FIG. 10. The feed enters the tube side of the first ODH reactor to give the flowing reaction mixture in the tubes. The reaction mixture flows through the first, second, and third cooling sections of the first ODH reactor. The reaction mixture discharges from the first ODH reactor from the tube side as effluent (first-reactor effluent) (analogous to effluent 808 of FIG. 8) for feed to the tube side of the second ODH reactor. The effluent may be the similar or same compositionally as the effluent 1008 of FIG. 10, but is typically at a lower temperature than the effluent 1008 as discharged because of the presence of the third cooling section of the first ODH reactor in FIG. 11. These two quench cooling sections can be characterized as in lieu of water quenching to cool down the reactor effluent of first ODH reactor and second ODH reactor before adding O₂.

In FIG. 11, oxygen gas may be injected into the first-reactor effluent, as in FIG. 10. However, injecting liquid water for a water quench into the first-reactor effluent as in FIG. 10 may be avoided because the third cooling section of the first ODH reactor cools the reaction mixture that discharges as the first-reactor effluent. In general, for cases of O₂ injection, water injection may be implemented if the inter-stage effluent temperature is relatively high. The three cooling techniques (water quenching, salt quenching, and HEX between two reactors) to give cooled inter-stage effluent before O₂ addition can be employed alone or in combination.

The first-reactor effluent plus the injected oxygen enters as feed to the second ODH reactor to give a reaction mixture flowing through the tube side. This reaction mixture flows in the tubes through the three cooling sections of the second ODH reactor and discharges as effluent (second-reactor effluent) from the second ODH reactor. Oxygen gas may be injected into the second-reactor effluent. The second-reactor effluent plus the injected oxygen enters as feed to the third ODH reactor to give a reaction mixture flowing through the tube side of the two cooling sections of the third ODH reactor. This reaction mixture may discharge from the tube side of the third ODH reactor as effluent (third-reactor effluent) that may be analogous to the effluent 108 of FIG. 1. This effluent may be cooled in an effluent heat exchanger with water as cooling medium, and the water vaporized into steam with heat from the effluent and the steam sent to the steam drum. In ODH reactor system 1100, six superheater heat exchangers (as depicted) superheat the steam discharged from the steam drum for distribution to a user(s).

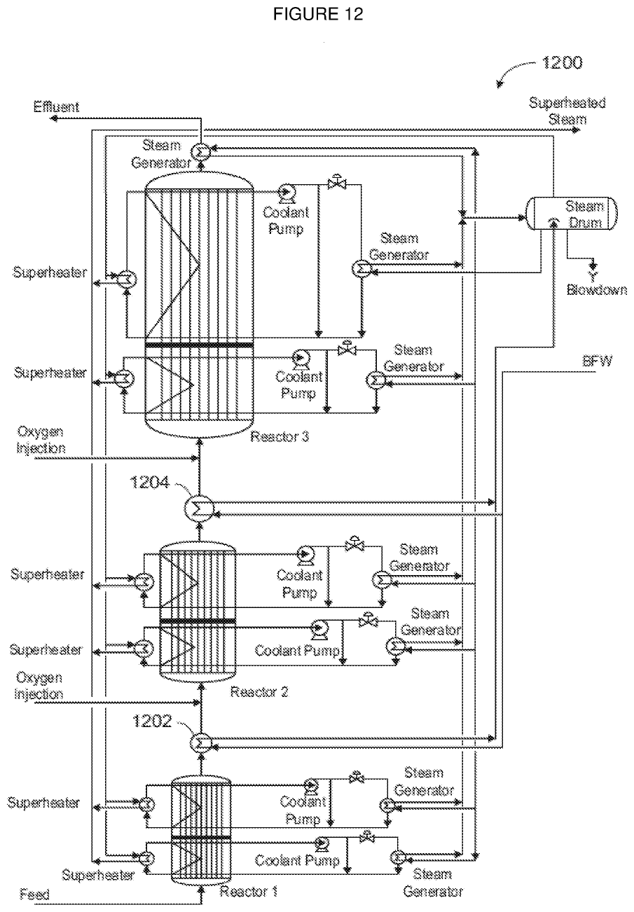

FIG. 12 is an ODH reactor system 1200 that is the same or similar as the ODH reactor system 1000 of FIG. 10, except that effluent heat exchangers 1202, 1204 (instead of an injected-water quench) cools the effluent discharged from the first ODH reactor and the second ODH reactor, respectively. An advantage of the effluent heat exchangers 1202, 1204 can be that the heat removed from the effluent can be recovered to preheat water (e.g., BFW) for steam generation. Conversely, for an injected-water quench, the removed heat may be lost in the vaporization of the injected water in the effluent.

For the ODH reactor system 1200 generally, refer to FIGS. 3-11 for a discussion of similar or same equipment and operation. The ODH reactor system 1200 may be the ODH reactor system 102 of FIG. 1. For comparison of examples of ODH reactor systems disclosed herein, an implementation of the ODH reactor system 1200 provides a basis for Configuration 13.

The effluent heat exchangers 1202, 1204 (e.g., shell-and-tube heat exchanger) may each cool the respective effluent with water (e.g., BFW) to a specified temperature, for example, in a range of 150° C. to 350° C. (or in the range of 200° C. to 300° C.). The effluent heat exchangers 1202, 1204 may receive the water (e.g., from a BFW pump) and heat the water (in the cooling of the effluent) that pre-heats the water for vaporization of the water in the steam drum. The water may be provided to the heat exchangers 1202, 1204 generally at the operating pressure (e.g., HP/VHP) of the steam drum. The water may flow from the pump (e.g., BFW pump) through the heat exchangers 1202, 1204 to the steam drum.

In FIG. 12, as discussed with respect to FIG. 10, the six coolant heat exchangers in the coolant supply systems, respectively, and the third-reactor effluent heat exchanger may vaporize water (e.g., BFW) (as cooling medium) from the steam drum to give steam sent to the steam drum. As also discussed, a liquid level of the water may be maintained in the steam drum. Steam may discharge from the steam drum for distribution to users. The steam after discharge from the steam drum may be superheated in superheater heat exchangers (as depicted) prior to distribution to users, as discussed with respect to preceding figures.

As discussed, for the process feed (e.g., 104 of FIG. 1) to the ODH reactor system (e.g., 102 of FIG. 1), dilution steam may be included with the ethane and oxygen gas to maintain the feed mixture outside of flammability limits.

FIG. 13 is an example of a flammability diagram 1300 for mixtures of ethane, oxygen, and steam at 300° C. and 500 kilopascals (kPa) absolute. The flammability diagram 1300 is a ternary plot with an ethane axis and associated horizontal grid lines 1302 for molar concentration of ethane in the mixture in mole percent (mol %), an oxygen axis with associated diagonal grid lines 1304 for molar concentration of oxygen gas in the mixture in mol %, and a steam axis with associated diagonal grid lines 1306 for molar concentration of oxygen gas in the mixture in mol %. The area (region) between the upper flammability limit (UFL) 1308 and the lower flammability limit (LFL) 1310 is a flammability zone meaning the mixture at those compositions in that area are flammable. The area of the plot outside of the flammability limits is not flammable and may be a desired zone of operation (outside of the flammable zone). The line 1312 represents a molar ratio of oxygen to ethane of 3.5 for combustion of all the ethane.

As a contingency in operation of an ODH reactor system, the maximum concentration of oxygen in the feed can be specified at a margin 1314 (e.g., 1-5 mol %) less than the oxygen concentration at the UFL 1308. As depicted, the margin 1314 is a line parallel to the line for the UFL 1308. The margin 1314 is an amount in oxygen mol % outside of the flammability zone—in particular, a margin outside of the upper flammability limit (UFL 1308). The oxygen concentration can be decreased from the oxygen concentration the UFL 1308 to the margin 1314 by increasing concentration of the dilution steam. Table 1 shows example target concentration of oxygen in the feed based on FIG. 13 (flammability diagram at 300° C. and 500 kPa). Margins 1314 considered as tabulated are no margin, 1 mol % less, 2 mol % less, 3 mol % less, 4 mol % less, and 5 mol % less.

TABLE 1
Oxygen Target in Mixture (Feed) Based on FIG. 13.

| Ethane | O₂ mol % | O₂ mol % | O₂ mol % | O₂ mol % | O₂ mol % | O₂ mol % |
|---|---|---|---|---|---|---|
| mol % | (no margin) | (<1 mol %) | (<2 mol %) | (<3 mol %) | (<4 mol %) | (<5 mol %) |
| 2-9.99 | 11 | 10 | 9 | 8 | 7 | 6 |
| 10-19.99 | 12 | 11 | 10 | 9 | 8 | 7 |
| 20-29.99 | 13 | 12 | 11 | 10 | 9 | 8 |
| 30-39.99 | 14 | 13 | 12 | 11 | 10 | 9 |
| 40-49.99 | 15 | 14 | 13 | 12 | 11 | 10 |
| 50-83 | 16 | 15 | 14 | 13 | 12 | 11 |

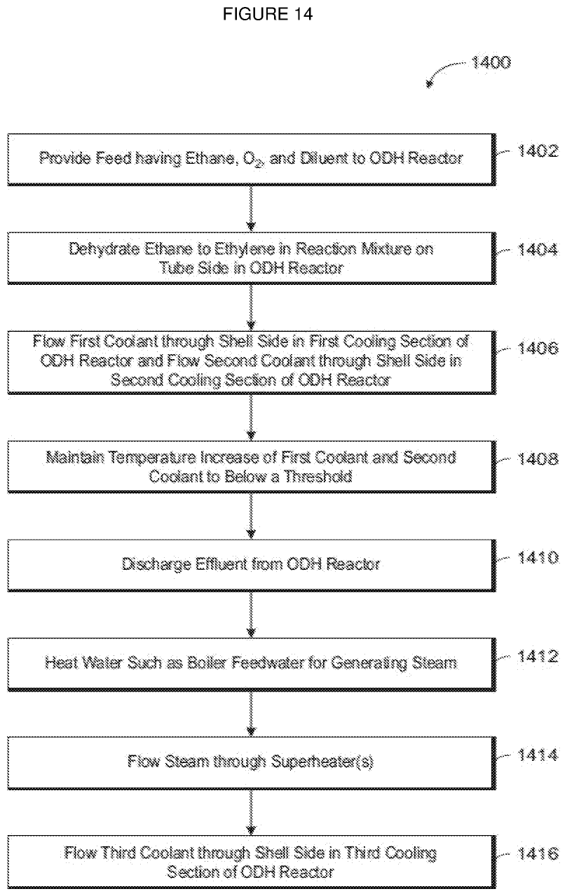

FIG. 14 is a method 1400 of operating an ODH reactor system having an ODH reactor. The ODH reactor is a multi-tubular reactor having a tube side and a shell side. The tube side may include tubes for flow of a reaction mixture. The shell side may include a volume in the reactor around (exterior to) the tubes for flow of coolant around the tubes. The ODH reactor includes a first cooling section and a second cooling section that may be separated by a barrier on the shell side. The second cooling section may be operationally downstream of the first cooling section with respect to the direction of flow of the reaction mixture flowing through the tube side. The ODH reactor may have the flow barrier on the shell side separating the first cooling section and the second cooling section such that the first coolant and the second coolant do not combine on the shell side.

At block 1402, the method includes providing feed including ethane, oxygen, and diluent (e.g., water as steam) to give the reaction mixture flowing through the tube side of the ODH reactor. The diluent can be water, CO₂, N₂, argon, helium, or methane, or any combinations thereof. The presence of diluent including these aforementioned diluents may maintain the feed outside of flammability limits. For example, the feed may include water (dilution steam) as diluent, thereby maintaining the feed outside of flammability limits. The providing of the feed to the tube side may be providing the feed to the inlet of tubes (of a tube bundle) of the ODH reactor. The tubes may have ODH catalyst disposed therein.

At block 1404, the method includes dehydrogenating ethane to ethylene in the reaction mixture (via ODH catalyst) on the tube side. The ODH reaction may include dehydrogenating ethane to ethylene in the tubes via the ODH catalyst in presence of the oxygen in the reaction mixture. Reactions of ethane and oxygen in the reaction mixture may include a first overall reaction that is dehydrogenating of the ethane to ethylene, a second overall reaction giving acetic acid (and water), a third overall reaction giving carbon monoxide (and water), and a fourth overall reaction giving carbon dioxide (and water). These four overall reactions may each be representations that have intermediate reactions embedded therein. These four reactions are simplified representations of overall reactions that can be characterized as yield type of reactions (overall reactions) with each having associated chains of reactions. The method may include specifying increasing ethylene selectivity by favoring the first overall reaction over the third overall reaction and the fourth overall reaction, wherein the first overall reaction consumes less stoichiometric amount of oxygen than each of the third overall reaction and the fourth overall reaction.

At block 1406, the method includes flowing a first coolant through the shell side in the first cooling section, thereby maintaining the reaction mixture (and/or the ODH catalyst) in the first cooling section at a first temperature, such as in ranges of 300° C. to 450° C., 350° C. to 450° C., 300° C. to 400° C., or 300° C. to 375° C. The first coolant may be, for example, molten salt. As appreciated by one of ordinary skill in the art, molten salt can be salt that is solid at standard temperature and pressure but enters the liquid phase due to elevated temperature. Molten salts may be a class of ionic liquids that are solid at ambient temperature. Molten salt (e.g., fluoride salts, chloride salts, nitrate salts, etc.) can be a heat transfer fluid employed as a coolant for an ODH reactor.

At block 1406, the method includes flowing a second coolant (e.g., molten salt) through the shell side in the second cooling section, thereby maintaining the reaction mixture (and/or the ODH catalyst) in the second cooling section at a second temperature, wherein the first temperature is lower than the second temperature. The second temperature may be, for example, in the range of 350° C. to 500° C. or in the range of 375° C. to 450° C. ODH reactors can have a third cooling section (e.g., FIG. 6) up to “n” cooling sections.

The method includes cooling the reaction mixture (and/or the ODH catalyst) to a first temperature in the first cooling section via the first coolant flowing through the shell side in the first cooling section and to a second temperature in the second cooling section via the second coolant flowing through the shell side in the second cooling section. The shell side is for flow of the first coolant and the second coolant around the tubes. The second coolant and the first coolant may both be molten salt and have the same composition.

The method may include specifying that the first temperature be lower than the second temperature to favor the dehydrogenating of ethane into ethylene over a reaction in the reaction mixture giving carbon dioxide and over a reaction in the reaction mixture giving carbon monoxide, thereby increasing ethylene selectivity. The maintaining of the first temperature to be lower than second temperature may be in response to specifying increasing ethylene selectivity and increases the ethylene selectivity, thereby reducing an amount of oxygen in the feed to reduce an amount of water in the feed. Moreover, present ODH reactors with more than two cooling sections may give more benefit on the performance of the reactions and better selectivity towards ethylene.

In a given cooling section, the temperature of the reaction mixture and the ODH catalyst may be substantially the same. The temperature difference between the reaction mixture and the ODH catalyst may be, for example, less than 1° C. or less than 2° C. The flow of coolant on the shell side cools both the reaction mixture and the ODH catalyst. At steady state, the temperature of the coolant may be similar to the temperature of the reaction mixture and the ODH catalyst.

At block 1408, the method includes maintaining temperature increase of the first coolant through the first cooling section at below a first threshold. The first threshold may be, for example, in a range of 2° C. to 8° C. The method includes maintaining temperature increase of the second coolant through the second cooling section at below a second threshold. The second threshold may be, for example, in a range of 2° C. to 8° C. These specified thresholds may dictate the amount of molten salt circulation.

The method may include specifying maintaining the temperature increase of the first coolant at below the first threshold and the temperature increase of the second coolant at below the second threshold to favor the dehydrogenating of ethane into ethylene over the reaction giving carbon dioxide reaction and over the reaction giving carbon monoxide, thereby increasing ethylene selectivity. This maintaining of the temperature increase of the first coolant at below the first threshold and the temperature increase of the second coolant at below the second threshold may be in response to specifying increasing ethylene selectivity and increases the ethylene selectivity, thereby reducing an amount of oxygen in the feed to reduce an amount of water in the feed.

At block 1410, the method may include discharging an effluent from the ODH reactor, the effluent comprising ethylene, acetic acid, water, carbon dioxide, and carbon monoxide. In particular, the method may include discharging the reaction mixture as the effluent from the ODH reactor.

At block 1412, the method may include heating water (e.g., BFW) with at least one of the first coolant discharged from the ODH reactor, the second coolant discharged from the ODH reactor, or the effluent discharged from the ODH reactor. In implementations, heating the water by at least one of the first coolant, the second coolant, or the effluent vaporizes the water, thereby generating steam from the water.

At block 1414, the method may include flowing the steam generated to a steam drum and from the steam drum through a superheater heat exchanger (e.g., one per cooling section of the ODH reactor). There may be one superheater heat exchanger per cooling section. There may be more than one superheater per cooling section in series and/or parallel. The superheater heat exchanger may be a heat exchanger (e.g., shell-and-tube heat exchanger) disposed adjacent to (but not mounted on) the ODH reactor. Coolant from the ODH reactor shell may be routed as a heating medium through the superheater heat exchanger. Alternatively, the superheater heat exchanger may be a supplemental tube bundle within the reactor shell or an add-on tube bundle (having a dedicated shell) mounted on the ODH reactor, and in which the dedicated shell shares coolant with the ODH reactor shell.

At block 1416, the method may include flowing a third coolant through the shell side in a third cooling section of the ODH reactor, thereby maintaining the reaction mixture on the tube side in the third cooling section at a third temperature, wherein the third temperature is lower than the second temperature. The third cooling section (if employed) is operationally downstream of the second cooling section in the flow direction of the reaction mixture and is separated from the second cooling section by a second flow barrier on the shell side. In implementations, the third cooling section can be a catalyst cooling section or a non-catalyst cooling. If a catalyst cooling section, the temperature of coolant may be the highest (greatest) among the three cooling sections. If a non-catalyst coolant section, e.g., coolant (salt) quenching section, the temperature of the reaction mixture and the coolant may be the least (lowest) among the three cooling sections. The non-catalyst cooling section (no catalyst but perhaps with the inert particle inside tubes) as the third cooling section (if employed) may be utilized to cool down the reaction mixture for discharge as the reactor effluent in lieu of utilizing a TLE to cool the effluent.

In accordance with block 1412 and block 1416, the method may include heating water (e.g., BFW) with at least one of the first coolant discharged from the ODH reactor, the second coolant discharged from the ODH reactor, the third coolant discharged from the ODH reactor, or the effluent discharged from the ODH reactor. In implementations, the tube side in the third cooling section does not include catalyst, and the water (e.g., BFW) is not heated with the effluent. In other implementations, the tube side in the third cooling section does not include catalyst, and the water (e.g., BFW) is heated with the effluent.

The method may include configuring the ODH reactor to have the tubes at or less than a specified diameter (e.g., nominal diameter, outside diameter, or inside diameter) in response to specifying increasing ethylene selectivity, thereby increasing the ethylene selectivity. The specified diameter to be at or less than, for instance, a diameter (e.g., nominal diameter, outside diameter, or inside diameter) of 1.5 inch or 1.25 inch, wherein the linear velocity of the reaction mixture in the tubes is in a range of 150 centimeters per second (cm/s) to 500 cm/s, and wherein the gas hourly space velocity of the reaction mixture through the ODH catalyst in the tubes is in a range of 1,500 hour⁻¹ (hr⁻¹) to 10,000 hr⁻¹.

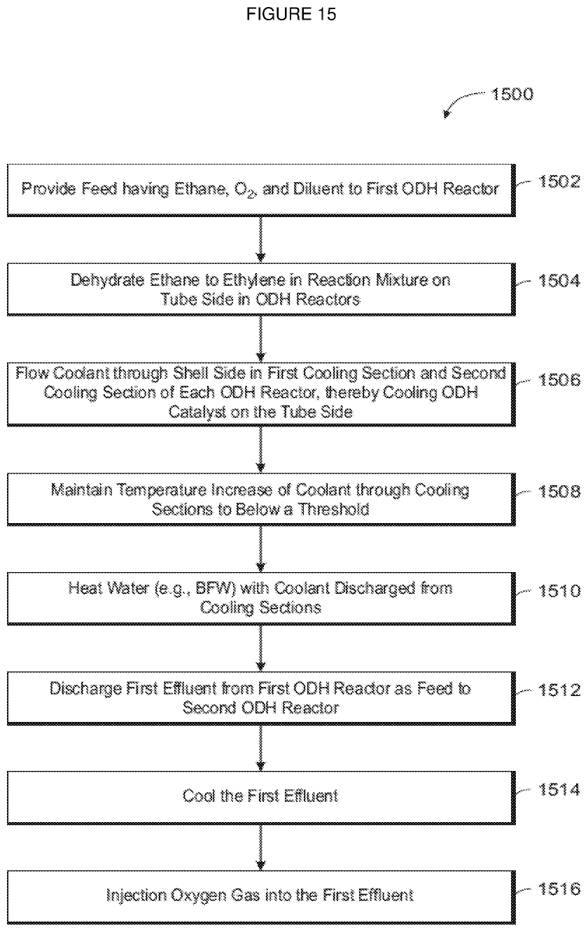

FIG. 15 is a method 1500 of operating an ODH reactor system having a first ODH reactor and a second ODH reactor disposed operationally in series and with each being a multi-tubular fixed bed reactor having a shell side and a tube side. The first ODH reactor and the second ODH reactor each have a first cooling section and a second cooling section. The first ODH reactor and the second ODH reactor may each have a flow barrier on the shell side separating the first cooling section and the second cooling section. For the first ODH reactor and the second ODH reactor, the second cooling section maybe operationally downstream of the first cooling section in the direction of flow of the reaction mixture flowing through the tube side and is separated from the first cooling section by the flow barrier on the shell side.

At block 1502, the method includes providing feed including ethane, oxygen (oxygen gas), and diluent (e.g., steam) to the first ODH reactor. Such may give a reaction mixture flowing through the tube side of the first ODH reactor.

At block 1504, the method includes dehydrogenating ethane to ethylene via ODH catalyst in a reaction mixture flowing on the tube side of each of the first ODH reactor and the second ODH reactor. Additional reactions may occur in the reaction mixture, as discussed.

At block 1506, the method includes flowing coolant (e.g., molten salt) through the shell side in the first cooling section and the second cooling section of each of the first ODH reactor and the second ODH reactor, thereby cooling the ODH catalyst on the tube side. Such may cool the reaction mixture and the ODH catalyst on the tube side of each of the first ODH reactor and the second ODH reactor. The shell side may be for flow of the coolant to cool ODH catalyst on the tube side. The cooling of the ODH catalyst may involve (1) maintaining the ODH catalyst (and/or the reaction mixture) on the tube side in the first cooling section of the first ODH reactor at a temperature lower than temperature of the ODH catalyst (and/or the reaction mixture) on the tube side in the second cooling section of the first ODH reactor, and (2) maintaining the ODH catalyst (and/or the reaction mixture) on the tube side in the first cooling section of the second ODH reactor at a temperature lower than temperature of the ODH catalyst (and/or the reaction mixture) on the tube side in the second cooling section of the second ODH reactor. The temperature of the ODH catalyst and the reaction mixture in the first cooling section of each of the first ODH reactor and the second ODH reactor may be, for example, in the range of 300° C. to 450° C. The temperature of the ODH catalyst and the reaction mixture in the second cooling section of each of the first ODH reactor and second ODH reactor may be, for example, in a range of 350° C. to 500° C. In a given cooling section, the temperature of the reaction mixture and the ODH catalyst may be substantially the same. The temperature difference between the reaction mixture and the ODH catalyst may be, for example, less than 1° C. or less than 2° C. At steady state, the temperature of the coolant may be similar to the temperature of the reaction mixture and the ODH catalyst.

At block 1508, the method includes maintaining temperature increase of the coolant through each of the first cooling section and the second cooling section of each of the first ODH reactor and the second ODH reactor to below a threshold. The threshold may be, for example, in a range of 2° C. to 8° C.

At block 1510, the method include heating water (e.g., BFW) with coolant discharged from at least one of the first cooling section of the first ODH reactor, the second cooling section of the first ODH reactor, the first cooling section of the second ODH reactor, or the second cooling section of the second ODH reactor, wherein heating the water vaporizes the water into steam or pre-heats the water for vaporization (vaporizing) of the water into steam in a steam drum.

At block 1512, the method includes discharging a first effluent from the first ODH reactor to the second ODH reactor. In particular, the method may include discharging the first effluent from the first ODH reactor as feed to the tube side of the second ODH reactor. The first effluent includes ethylene, acetic acid, water, carbon dioxide, carbon monoxide, and unreacted ethane.

At block 1514, the method includes cooling the first effluent. For example, the method may include injecting liquid water into the first effluent, thereby vaporizing the liquid water via heat from the first effluent to cool the first effluent. In lieu of (or in addition to) the liquid water injection, the method may include cooling the first effluent by a heat exchanger (e.g., operationally disposed between the first reactor and the second reactor) with boiler feedwater as cooling medium, thereby vaporizing the boiler feedwater into steam or heating the boiler feedwater for vaporizing the boiler feedwater into steam in a steam drum.

In lieu of (or in addition to) the liquid water injection and/or the heat exchanger for cooling the first effluent, the method may include an indirect or upstream cooling of the first effluent. In other words, the first ODH reactor may have a third cooling section (e.g. not having ODH catalyst in the tubes) to cool the reaction mixture in the tubes prior to discharge of the reaction mixture as the first effluent. Thus, for cooling the first effluent, the method may involve flowing coolant through the shell side in a third cooling section of the first ODH reactor, thereby cooling the reaction mixture on the tube side in the third cooling section, wherein the third cooling section is operationally downstream of the second cooling section of the first ODH reactor in the flow direction of the reaction mixture and is separated from the second cooling section by a second flow barrier on the shell side. In implementations, the tube side in the third cooling section (if employed) (in which the reaction mixture flows) of the first ODH reactor does not include catalyst. In implementations, the tubes in the third cooling section may have non-catalyst particles with relatively high thermal conductivity to facilitate or increase the heat removal.

At block 1516, the method includes injecting oxygen into the first effluent. In particular, the method may include injecting oxygen gas into the first effluent between the first ODH reactor and the second ODH reactor.

The method includes discharging a second effluent from the second ODH reactor. The second effluent includes ethylene, acetic acid, water, carbon dioxide, and carbon monoxide.

Congruent with block 1510, the method may include generating steam via heat from at least one of the coolant discharged from the first ODH reactor, the coolant discharged from the second ODH reactor, the first effluent discharged from the first ODH reactor, or the second effluent discharged from the second ODH reactor. The method may include flowing the steam through another bundle of tubes (not having ODH catalyst) associated with the first ODH reactor or the second ODH reactor, or both, to heat the steam with the coolant flowing on the shell side, thereby superheating the steam. The tubes may be in a separate shell-and-tube heat exchanger adjacent the ODH reactor and that receives/returns coolant (as heating medium) from/to the ODH reactor shell. Alternatively, the tubes may be a supplemental (add-on) bundle of tubes within the ODH reactor shell, or within a dedicated shell (adjacent to or mounted on the ODH reactor) that receives (and returns) a slipstream of coolant (molten salt) from the ODH reactor shell. The steam (e.g., at about saturation) may flow from a steam drum through these additional tubes. The superheating of the steam may be to increase the temperature of the steam to above the saturation temperature of the steam.

To reduce DS consumption, the amount of oxygen consumption should be reduced. In considering the four reactions listed above, the least oxygen demand is for ethylene production (0.5 mole O₂ per mole of ethane) and the highest oxygen demand is for CO₂ generation (3.5 mole O₂ per mole of ethane). Therefore, to reduce O₂ consumption, the selectivity towards ethylene should be increased. Further, increasing selectivity of acetic acid is more desirable than formation of CO or CO₂.

The ODH reactor design (and operation) can be configured to achieve higher ethylene selectivity (and higher acetic acid selectivity). The specified ODH reactor design is multi-tubular fixed bed reactor in which the tubes are filled with catalyst. In operation, process gas passes through those tubes, and coolant flows on the shell side removing the heat of reaction. Molten salt is specified as the coolant in the Examples below due to temperature of operation, which is generally in the range of 300° C. to 500° C. Molten salt can give higher stability at high temperature and better heat transfer coefficient compared to other heat transfer fluids generally.

Configurations 1-13 are presented as implementations and not intended to limit the present techniques. Alternate or other variations are applicable as indicated in the overall disclosure herein. Specific example data are applied for Configurations 1-13 in the Examples below. Other example data are applicable for Configurations 1-13. In the discussion of Configurations 1-13 for an ODH reactor system, a reactor may be referred to as a stage. In particular, a single-reactor system may be referred to as a single stage reactor system or 1-stage reactor system, and a multi-reactor system may be referred to as a multi-stage reactor system (e.g., 2-stage reactor system, 3-stage reactor system, etc.). For instance, a 2-stage reactor system is an ODH reactor system having two ODH reactors operationally in series. A 3-stage reactor system in an ODH reactor system having three ODH reactors operationally in series. The phrase “between stages” or the word “inter-stage” means between ODH reactors. Between the first stage and the second stage means between the first ODH reactor and the second ODH reactor. Between the second stage and the third stage means between the second ODH reactor and the third ODH reactor. An effluent heat exchanger that cools effluent discharged from the reactor in a single-reactor system or from a terminal (final) reactor in a multi-reactor system may be labeled as a transfer line exchanger (TLE). Inter-stage cooling of effluent may be provided by (1) a water quench via liquid water injection, (2) by a heat exchanger with water as coolant (this option represented by acronym HEX), or (3) by a salt quench (with coolant as molten salt) of the reaction mixture in a reactor terminal cooling section prior to discharge of the inter-stage effluent from the reactor. An HEX of final effluent (not inter-stage) may be labeled as TLE. A cooling section in an ODH reactor may be labeled as a catalyst cooling section for cooling sections in which the tubes have catalyst (ODH catalyst). A catalyst cooling section may be utilized to cool both the reaction mixture and the ODH catalyst on the tube side. For a cooling section in which the tubes do not have catalyst (ODH catalyst) (but may have inert particles disposed in the tubes), the cooling section may be characterized as non-catalyst cooling section that cools the reaction mixture on the tube side. The coolant as molten salt may be referred to as salt for shorthand.

Remarks and bases regarding the thirteen example configurations (Configurations 1-13) of the ODH reactor system are presented below. Implementations of the ODH reactor systems in FIGS. 3-12 provide bases for Configurations 1-13: Configuration 1 (FIG. 3), Configuration 2, 3, 4, and 5 (FIG. 4), Configuration 6 (FIG. 5), Configuration 7 (FIG. 6), Configuration 8 (FIG. 7), Configuration 9 (FIG. 8), Configuration 10 (FIG. 9), Configuration 11 (FIG. 10), Configuration 12 (FIG. 11), and Configuration 13 (FIG. 12). However, the respective figures are not limited by the specific implementations in the configurations presented. The figures provide a general bases regarding number of reactors, number of cooling sections, and heating of water. In contrast, specific implementations, e.g., regarding tube size (diameter), coolant as molten salt, values for temperature or temperature increase, etc., for the configurations do not limit the figures. Initial remarks as a summary regarding the Configurations 1-13 are given immediately below.

Configuration 1 is a single reactor (1-stage) with one catalyst cooling section (1-section), 1-inch tubes, and less than 5° C. temperature rise on coolant side. The coolant is utilized to generate HP/VHP steam and superheat the generated HP/VHP steam (500 psig to 1700 psig). The reactor effluent can be cooled down against BFW (that pre-heats or vaporizes the BFW for HP/VHP steam generation) in a TLE after the reactor.

Configurations 1, 2, and 7 are 1-stage with 1-, 2- or 3-catalyst cooling sections, respectively. The number of catalyst cooling sections can be extended to n-sections, which may give better performance in reactions.

Configurations 2 and 3 are 1-stage with 2-catalyst cooling sections. The temperature rise on coolant side in the 2-catalyst cooling sections is less than 5° C. and about 15° C., respectively. Other values for temperature rise of the coolant can be implemented. A 2-8° C. temperature rise (e.g., 5° C.) may be more favorable than higher values such as over 8° C. (e.g., 15° C.) in terms of reaction performance associated with coolant flow rate. More delta T may mean less coolant (e.g., molten salt) circulation that results in lower Reynolds number (Re) and thus lower heat transfer coefficient (HTC). A significant parameter to improve ODH reactor performance (higher ethylene selectivity and lower CO/CO₂ selectivity) is heat removal capability.

Configurations 2, 4, and 5 are 1-stage with 2-catalyst cooling sections. Configurations 2, 4, and 5 have a diameter of the tubes of 1 inch, 1.25 inch, and 1.5 inch, respectively. Other tube sizes can be implemented. In general, the smaller tubes may have better (increased) heat removal capability due to higher Re inside the tubes and thus higher HTC which may result in better reactor performance.

Configuration 2 is 1-stage with 2-catalyst cooling sections and with HP/VHP steam generation in a TLE after the reactor. Configuration 6 is 1-stage with 2-catalyst cooling sections, and a third cooling section (non-catalyst cooling section with inert particles in the tubes) as a low-temperature cooling section in which the tube-side reaction mixture (gas) is cooled (temperature drop) and the coolant (molten salt) pre-heats vaporizes BFW to generate HP/VHP steam. A difference between Configuration 2 (having TLE) and Configuration 6 (coolant quench section) is TLE versus coolant (e.g., molten salt) quench.

Configuration 8 is a 2-stage reactor system with 2-catalyst cooling sections for each stage and with oxygen gas (O₂) inter-stage injection. Liquid water is injected at inter-stage to evaporate and use the latent heat of evaporation to reduce the reactor effluent temperature from first stage (before O₂ inter-stage injection) to a temperature in the range of 200° C. to 300° C. This may further advance reduction of risk of autoignition of hydrocarbon and other combustible gases. The 2-stage reactor system splits the O₂ between two stages. This way the first stage receives less O₂ than in a 1-stage reactor system and thus requires much less dilution steam (DS) compared to 1-stage reactor system configuration.

Configuration 9 is 2-stage reactor system with 2-catalyst cooling sections for each stage and with O₂ inter-stage injection. Similar to the single reactor in Configuration 6, the first reactor (stage) in Configuration 9 has an inert (non-catalyst) cooling section with coolant (cold molten salt as salt quench) at the end of first reactor to cool down the tube-side gas before the O₂ injection. The coolant (colder molten salt) pre-heats BFW or generates HP/VHP steam. The first reactor has three cooling sections (molten salt cooling sections): two to cool down the catalytic reaction and third to cool down the tube-side gas before exiting the reactor.

Configuration 10 is 2-stage reactor system with 2-catalyst cooling sections for each stage and with O₂ inter-stage injection. Instead of water quench or molten salt quench, a heat exchanger (HEX) cools down the reactor effluent from first stage and recovers the heat to pre-heat BFW or generate HP/VHP steam. As discussed herein this quench context, the phrase salt quench refers to molten salt quench (the coolant molten salt in the shell of the ODH reactor).

Configurations 11, 12, and 13 are 3-stage reactor system with 2-catalyst cooling section for each stage and with O₂ inter-stage injection. Inter-stage can employ water quench, salt quench, or HEX, or for reactor effluent cooling before O₂ injection as previously discussed (Configurations 8, 9, and 10). Combinations of these techniques may be employed. Applicability can be extended for n-stage reactor.

For the specific implementations in Configurations 1-7, some features for a 1-stage reactor system include O₂ target is 6-10 mol % in feed, water/O₂ molar ratio is 6-12 in feed, linear velocity on tube side is 150 cm/s to 500 cm/s, and gas hourly space velocity (GHSV) through ODH catalyst on tube side is 1,500-10,000 hr⁻¹. The features may depend on temperature and pressure. The features may be considered at feed conditions (e.g., 200-350° C. and 300-600 kPa absolute) to the ODH reactor. Observations for Configurations 1-7 (1-stage reactor system) include: (a) more than 1-catalyst cooling section would improve ethylene selectivity and reduce the overall feed flow to the reactor and thus result in the reactor being smaller; (b) the smaller tube diameter is generally better for ethylene selectivity as smaller diameter improves (increases) the heat transfer coefficient (HTC) and heat removal, and thus prevents or reduces occurrence of spikes in the peak temperature (the higher peak temperature would generally result in more unwanted reactions); (c) more temperature rise in coolant side may have negative impact on the heat removal and the size of the reactor, due to likely less coolant (salt) circulation that could result in less HTC; and (d) having a salt quench section is more effective than TLE for overall size of the reactor (however, a salt quench adds complexity of the reactor design, i.e., one more cooling section [molten salt section]).

For the specific implementations in Configurations 8-10 (2-stage reactor system), some features for the 2-stage reactor systems include O₂ target is 6-10 mol % for feed to the first stage reactor, O₂ target is 6-10 mol % for feed to the second stage reactor, water/O₂ molar ratio for the first stage reactor is 1.5-3.5, linear velocity for the first stage reactor is 250 cm/s to 600 cm/s and for the second stage is 100 cm/s to 400 cm/s, GHSV for both stages combined is 2,000-6,000 hr⁻¹, GHSV for stage one reactor is 5,000-30,000 hr⁻¹ and for the second stage reactor is 2,000-10,000 hr⁻¹. These features may be affected by feed temperature and feed pressure, which are considered for feed to the first stage at 200-350° C. and 300-600 kPa absolute and for feed to the second stage at 150-300° C. and 300-500 kPa absolute. The tube-side temperature (including reaction mixture and/or ODH catalyst) (and generally the shell-side temperature) in the first cooling section of each stage (reactor) may be, for example, in the range of 300-450° C. The tube-side temperature (including reaction mixture and/or ODH catalyst) (and generally the shell-side temperature) in the second cooling section of each stage (reactor) may be, for example, in the range of 350-500° C. An observation for 2-stage reactor system is for HEX at inter-stage or salt quench of discharging reaction mixture at inter-stage, the first reactor is larger and the second reactor smaller than if water quench is employed at inter-stage. In general, the second reactor is typically bigger than the first reactor, including when water quench is employed. Nevertheless, in implementations, for HEX and salt quenching versus water quench, the first reactor may be larger for HEX and salt quench than water quench. In implementations, the second reactor may be smaller for HEX and salt quench than water quench.

For the specific implementations in Configurations 11-13 (3-stage reactor system), the 3-stage reactor systems include O₂ target of 6-10 mol % for feed to each of the first stage reactor, second stage reactor, and the third stage reactor. The vast majority of the O₂ per stage should be consumed. The O₂ at the discharge of each stage may be, for example, less than 0.1 mol %. The water/O₂ molar ratio for the first stage reactor is 0.5-2.5 and with generally no water/O₂ molar ratio targets for subsequent stage as no DS is typically added inter-stage. If there is water-quenching inter-stage, the purpose is not dilution but rather quenching the effluent. For quenching, an adequate amount of water may be added to lower the effluent temperature down, for example, to 200-300° C. Additional features of these 3-stage reactor systems include linear velocity is 250-700 cm/s for the first stage reactor, 100-450 cm/s for the second stage reactor, and 50-350 cm/s for the third stage reactor, and GHSV for three stages combined is 2,000-6,000 hr⁻¹. GHSV is 5,000-60,000 hr⁻¹ for stage one reactor, 5,000-30,000 hr⁻¹ for the second stage reactor, and 2,000-10,000 hr⁻¹ for the third stage reactor. These features may be affected by feed temperature and feed pressure, which are considered for the first stage reactor at 200-350° C. and 300-600 kPa absolute, for the second stage reactor at 150-300° C. and 200-500 kPa absolute, and for the third stage reactor at 150-300° C. and 200-500 kPa absolute.

For the specific implementations in Configurations 8-13 (2-stage reactor systems or 3-stage reactor system), observations for the multi-reactor systems include for salt quench or HEX at inter-stage, a larger first reactor, larger second reactor, and smaller third reactor are employed than if water quench is implemented inter-stage. From 2-stage to 3-stage, the sizes of the reactors may change. In general, the size of first reactor <second reactor <third reactor < . . . <nth reactor. If quench water is employed, the size of last reactor is bigger compared to HEX and salt quench. If quench water is employed, the size of reactors before the last one generally are smaller compared to HEX and salt quenching cases. Additional observations for the multi-reactor systems include (a) less O₂ to the first stage and much less DS demand compared to 1-stage (single reactor) system; (b) first reactors smaller than terminal (final) reactor (and smaller than single reactor in 1-stage system); (c) more stages result in slightly less ethylene selectivity (ethylene selectivity for 1-stage >2-stage >3-stage > . . . >n-stage); (d) more stages results in higher acetic acid (AA) selectivity (AA Selectivity for 1-stage <2-stage <3-stage < . . . <n-stage); (e) more stages result in lower CO/CO₂ selectivity (CO/CO₂ selectivity for 1-stage >2-stage >3-stage > . . . >n-stage); (f) salt quench and HEX at inter-stage can contribute to steam generation, whereas water quench generally does not with the liquid water evaporating and no heat recovery; (g) for salt Quench or HEX at inter-stage, more DS is implemented at the mixed feed to first reactor than water quench (because at water quench, liquid water is added to 2nd or 3rd reactor which helps with dilution); (h) any combination of salt quench/HEX/water quench can be employed for inter-stage cooling of reactor effluent; and (i) with the salt quench or HEX, more stages result in more HP/VHP steam generation. This disclosure captures 1-, 2-, 3-stage reactor system which can be extended to n-stage reactor system. This disclosure captures 1-, 2-, or 3-catalyst cooling sections (which can be extended to n-catalyst cooling sections) for each stage reactor.

An embodiment is a method of operating an ODH reactor system, including providing feed including ethane, oxygen, and diluent (e.g., steam) to give a reaction mixture flowing through a tube side of an ODH reactor that is a multi-tubular reactor having the tube side and a shell side, wherein the ODH reactor includes a first cooling section and a second cooling section. The method includes dehydrogenating ethane to ethylene (in the reaction mixture) via ODH catalyst on the tube side. The method includes flowing a first coolant (e.g., molten salt) through the shell side in the first cooling section, thereby maintaining the reaction mixture in the first cooling section at a first temperature (e.g., in a range of 300° C. to 450° C.). The method includes flowing a second coolant (e.g., molten salt) through the shell side in the second cooling section, thereby maintaining the reaction mixture in the second cooling section at a second temperature (e.g., in a range of 350° C. to 500° C.), wherein the first temperature is lower than the second temperature. The ODH reactor may have a flow barrier on the shell side separating the first cooling section and the second cooling section such that the first coolant and the second coolant do not combine on the shell side. The second cooling section may be operationally downstream of the first cooling section in the flow direction of the reaction mixture. Again, the second cooling section may be separated from the first cooling section by a flow barrier on the shell side. The method includes maintaining temperature increase of the first coolant through the first cooling section at below a first threshold (e.g., in a range of 2° C. to 8° C.), and maintaining temperature increase of the second coolant through the second cooling section at below a second threshold (e.g., in a range of 2° C. to 8° C.). The method may include discharging an effluent from the ODH reactor. The effluent may include ethylene, acetic acid, water, carbon dioxide, and carbon monoxide. The method may include heating water with at least one of the first coolant discharged from the ODH reactor, the second coolant discharged from the ODH reactor, or the effluent discharged from the ODH reactor. In implementations, this heating of the water (e.g., BFW) by at least one of the first coolant, the second coolant, or the effluent vaporizes the water, thereby generating steam from the water. If so, the method may include flowing the steam through tubes of heat exchangers to heat the steam with the first coolant or the second coolant, or both, flowing on the shell side, thereby superheating the steam, wherein the water comprises boiler feedwater, and discharging the steam as superheated from the heat exchangers.

The method may include flowing a third coolant through the shell side in a third cooling section of the ODH reactor, thereby maintaining the reaction mixture on the tube side in the third cooling section at a third temperature, wherein the third temperature is lower than the second temperature, wherein the third cooling section is operationally downstream of the second cooling section in the flow direction of the reaction mixture and is separated from the second cooling section by a second flow barrier on the shell side. The method may include heating water with at least one of the first coolant discharged from the ODH reactor, the second coolant discharged from the ODH reactor, the third coolant discharged from the ODH reactor, or the effluent discharged from the ODH reactor. In implementations, the tube side in the third cooling section does not include catalyst, and in certain implementations, the water is not heated with the effluent.

Another embodiment is method of operating ODH reactor system, including providing feed including ethane and oxygen into tubes of an ODH reactor, the tubes having ODH catalyst disposed therein, wherein the feed includes water (e.g., in the form of steam) as diluent, thereby maintaining the feed outside of flammability limits. The method includes dehydrogenating ethane to ethylene in the tubes via the ODH catalyst in presence of the oxygen in a reaction mixture. The ODH reactor is a multi-tubular fixed bed reactor with a tube side having the tubes for flow of the reaction mixture and a shell side. The ODH reactor has a first cooling section and a second cooling section operationally downstream of the first cooling section in flow direction of the reaction mixture. The first cooling section and the second cooling section may be segregated by a flow barrier on the shell side. The method includes cooling the ODH catalyst to a first temperature (e.g., in a range of 300° C. to 450° C.) in the first cooling section via a first coolant flowing through the shell side in the first cooling section and to a second temperature (e.g., in a range of 350° C. to 500° C.) in the second cooling section via a second coolant flowing through the shell side in the second cooling section, wherein the first temperature is lower than the second temperature. The method may include maintaining the first temperature to be lower than second temperature in response to specifying increasing ethylene selectivity and increases the ethylene selectivity, thereby reducing an amount of oxygen in the feed to reduce an amount of water in the feed.

The method may include specifying that the first temperature be lower than the second temperature to favor the dehydrogenating of ethane into ethylene over a reaction in the reaction mixture giving carbon dioxide and over a reaction in the reaction mixture giving carbon monoxide, thereby increasing ethylene selectivity. The method includes maintaining temperature increase of the first coolant through the first cooling section at below a first threshold (e.g., in a range of 2° C. to 8° C.), and maintaining temperature increase of the second coolant through the second cooling section at below a second threshold (e.g., in a range of 2° C. to 8° C.). The method may include specifying maintaining the temperature increase of the first coolant at below the first threshold and the temperature increase of the second coolant at below the second threshold to favor the dehydrogenating of ethane into ethylene over the reaction giving carbon dioxide and over the reaction giving carbon monoxide, thereby increasing ethylene selectivity. The maintaining of the temperature increase of the first coolant at below the first threshold and the temperature increase of the second coolant at below the second threshold may be in response to specifying increasing ethylene selectivity and increases the ethylene selectivity, thereby reducing an amount of oxygen in the feed to reduce an amount of water in the feed.

The reactions of the ethane and the oxygen in the reaction mixture may include a first overall reaction including the dehydrogenating of the ethane to ethylene, a second overall reaction giving acetic acid, a third overall reaction giving carbon monoxide, and a fourth overall reaction giving carbon dioxide. The method may include specifying increasing ethylene selectivity by favoring the first overall reaction over the third overall reaction and the fourth overall reaction, wherein the first overall reaction consumes less stoichiometric amount of oxygen than each of the third overall reaction and the fourth overall reaction.

The method may include discharging the reaction mixture as effluent from the ODH reactor, wherein the effluent includes ethylene, acetic acid, water, carbon dioxide, carbon monoxide, and unreacted ethane. The method may include heating boiler feedwater with at least one of the first coolant, the second coolant, or the effluent. In implementations, the heating the boiler feedwater vaporizes the boiler feedwater, thereby generating steam from the boiler feedwater. The method may include configuring the ODH reactor to have the tubes at or less than a specified diameter in response to specifying increasing ethylene selectivity, thereby increasing the ethylene selectivity. The specified diameter may be, for instance, an diameter (e.g., nominal diameter, outside diameter, or inside diameter) of 1.25 inch, wherein linear velocity of the reaction mixture in the tubes is in a range of 150 cm/s to 500 cm/s, and wherein gas hourly space velocity of the reaction mixture through the ODH catalyst in the tubes is in a range of 1,500 hour⁻¹ (hr⁻¹) to 10,000 hr⁻¹.

Yet another embodiment is an ODH reactor system including an ODH reactor (a multi-tubular fixed bed reactor) having a first cooling section and a second cooling section separated by a flow barrier on a shell side. The ODH reactor includes a tube side having ODH catalyst to receive feed comprising ethane, oxygen, and steam to dehydrogenate ethane into ethylene in a reaction mixture and discharge an effluent including ethylene, acetic acid, water, carbon dioxide, carbon monoxide, and unreacted ethane. The steam in the feed may act as a diluent to place the feed outside of flammability limits. The ODH reactor may be configured to generate acetic acid in the reaction mixture on the tube side. To dehydrogenate ethane into ethylene may be a first overall reaction of ethane with oxygen on the tube side. The ODH reactor as configured may give reactions of ethane with oxygen in the reaction mixture on the tube side including a second overall reaction giving acetic acid, a third overall reaction giving carbon monoxide, and a fourth overall reaction giving carbon dioxide.

The second cooling section may be operationally downstream of the first cooling section with respect to flow of the reaction mixture. The shell side is configured to receive a first coolant (e.g., molten salt) into the first cooling section to maintain temperature of the ODH catalyst on the tube side in the first cooling section at a first temperature (e.g., in a range of 300° C. to 450° C.) and receive a second coolant (e.g., molten salt) into the second cooling section to maintain temperature of the ODH catalyst on the tube side in the second cooling section at a second temperature (e.g., in a range of 350° C. to 500° C.), wherein the first temperature is lower than the second temperature. Again, the second cooling section may be operationally downstream of the first cooling section in the flow direction of the reaction mixture. The ODH reactor system includes a first-coolant supply system having a pump to provide the first coolant to the first cooling section and maintain temperature increase of the first coolant through the first cooling section to below a first threshold (e.g., in a range of 2° C. to 8° C.). The ODH reactor system includes a second-coolant supply system having a pump to provide the second coolant to the second cooling section and maintain temperature increase of the second coolant through the second cooling section to below a second threshold (e.g., in a range of 2° C. to 8° C.). The ODH reactor system includes a first heat exchanger to heat first water (e.g., BFW) with the first coolant for steam generation of the first water. The steam generation may involve the first heat exchanger configured to vaporize the first water into steam. The ODH reactor system may include a steam drum to receive the steam from the first heat exchanger and discharge the steam. Alternatively, the first heat exchanger heating the first water may be the first heat exchanger configured to heat the first water to pre-heat the first water for vaporization of the first water in the steam drum.

The ODH reactor system may include a second heat exchanger to heat second water (e.g., BFW) with the second coolant discharged from the second cooling section for steam generation of the second water. The ODH reactor system may include a third heat exchanger to heat third water (e.g., BFW) with the effluent for steam generation of the third water. Lastly, the ODH reactor may have a third cooling section operationally downstream of the second cooling section with respect to flow of the reaction mixture to receive a third coolant into the shell side. The third cooling section may be separated from the second cooling section by a second flow barrier on a shell side.

Yet another embodiment is a method of operating an ODH reactor system. The ODH reactor system includes a first ODH reactor and a second ODH reactor each being a multi-tubular fixed bed reactor having a shell side and a tube side. The first ODH reactor and the second ODH reactor each have a first cooling section and a second cooling section. The first ODH reactor and the second ODH reactor may each have a flow barrier on the shell side separating the first cooling section and the second cooling section. The second ODH reactor is disposed in series operationally downstream of the first ODH reactor. The method included providing feed including ethane, oxygen, and diluent (e.g., steam) to the first ODH reactor, and dehydrogenating ethane to ethylene via ODH catalyst in a reaction mixture flowing on the tube side of each of the first ODH reactor and the second ODH reactor. The method includes flowing coolant (e.g., molten salt) through the shell side in the first cooling section and the second cooling section of each of the first ODH reactor and the second ODH reactor, thereby cooling the reaction mixture and the ODH catalyst on the tube side. The method includes maintaining temperature increase of the coolant through each of the first cooling section and the second cooling section of each of the first ODH reactor and the second ODH reactor to below a threshold (e.g., 2° C. to 8° C.). The cooling of the reaction mixture may include: maintaining the reaction mixture on the tube side in the first cooling section of the first ODH reactor at a temperature lower than temperature of the reaction mixture on the tube side in the second cooling section of the first ODH reactor; and maintaining the reaction mixture on the tube side in the first cooling section of the second ODH reactor at a temperature lower than temperature of the reaction mixture on the tube side in the second cooling section of the second ODH reactor. In implementations, the temperature of the reaction mixture in the first cooling section of each of the first ODH reactor and the second ODH reactor is in a range of 300° C. to 450° C., and the temperature of the reaction mixture in the second cooling section of each of the first ODH reactor and second ODH reactor is in a range of 350° C. to 500° C.

The method includes discharging a first effluent from the first ODH reactor to the second ODH reactor, the first effluent including ethylene, acetic acid, water, carbon dioxide, carbon monoxide, and unreacted ethane. The method includes injecting oxygen into the first effluent. The method may include cooling the first effluent upstream of injecting the oxygen into the first effluent or cooling the reaction mixture in a third cooling section in the first ODH reactor that discharges as the first effluent from the first ODH reactor, or a combination thereof. The method may include injecting liquid water into the first effluent, thereby vaporizing the liquid water via heat from the first effluent to cool the first effluent. The method may include cooling the first effluent by a heat exchanger with boiler feedwater as cooling medium, thereby vaporizing the boiler feedwater into steam or heating the boiler feedwater for vaporizing the boiler feedwater into steam in a steam drum.

The method may include heating water with coolant discharged from at least one of the first cooling section of the first ODH reactor, the second cooling section of the first ODH reactor, the first cooling section of the second ODH reactor, or the second cooling section of the second ODH reactor, wherein heating the water vaporizes the water into steam or pre-heats the water for vaporization of the water into steam in a steam drum. For the first ODH reactor and the second ODH reactor, the second cooling section can be defined as operationally downstream of the first cooling section in flow direction of the reaction mixture and is separated from the first cooling section by a flow barrier on the shell side. The method may include flowing coolant through the shell side in a third cooling section of the first ODH reactor, thereby cooling the reaction mixture on the tube side in the third cooling section, wherein the third cooling section is operationally downstream of the second cooling section of the first ODH reactor in the flow direction of the reaction mixture and is separated from the second cooling section by a second flow barrier on the shell side. In implementations, the tube side in the third cooling section of the first ODH reactor flows does not include catalyst. The method may include discharging a second effluent from the second ODH reactor, the second effluent including ethylene, acetic acid, water, carbon dioxide, and carbon monoxide. The method may include generating steam via heat from at least one of the coolant discharged from the first ODH reactor, the coolant discharged from the second ODH reactor, the first effluent discharged from the first ODH reactor, or the second effluent discharged from the second ODH reactor. Lastly, the method may include heating the steam with coolant from the first ODH reactor or the second ODH reactor, or both, thereby superheating the steam.

Yet another embodiment is method of operating an ODH reactor system, including providing feed including ethane, oxygen, and diluent to a first ODH reactor having a tube side and a shell side to give a first reaction mixture flowing through the tube side. The feed may have the diluent (e.g., steam) to maintain the feed outside of flammability limits. The method includes dehydrogenating, via ODH catalyst on the tube side, ethane to ethylene in the first reaction mixture flowing through the tube side, wherein the first ODH reactor has a first cooling section and a second cooling section operationally downstream of the first cooling section. In implementations, the first cooling section and the second cooling section are separated by a flow barrier on the shell side. The method includes flowing a first coolant through the shell side in the first cooling section, thereby maintaining the ODH catalyst in the first cooling section at a first temperature. The method includes flowing a second coolant through the shell side in the second cooling section, thereby maintaining the ODH catalyst in the second cooling section at a second temperature. The method includes specifying the first temperature be less than the second temperature to favor ethylene selectivity in the first reaction mixture in the first cooling section. The method may include specifying temperature increase of the first coolant through the first cooling section be below a first threshold to favor ethylene selectivity in the first reaction mixture; and specifying temperature increase of the second coolant through the second cooling section be below a second threshold to favor ethylene selectivity in the first reaction mixture. The method may include maintaining temperature increase of the first coolant through the first cooling section below a first threshold, wherein the first temperature is less than the second temperature; and maintaining temperature increase of the second coolant through the second cooling section below a second threshold. The method may include discharging the first reaction mixture from the tube side as a first effluent from the first ODH reactor to a second ODH reactor to give a second reaction mixture flowing through the second ODH reactor.

The method may include cooling the first effluent by injecting liquid water into the first effluent or by a heat exchanger with boiler feedwater as cooling medium. The method may include injecting oxygen into the first effluent, wherein the first effluent as discharged from the first ODH reactor includes ethylene, acetic acid, water, carbon dioxide, carbon monoxide, and unreacted ethane. The method may include flowing third coolant through the shell side in a third cooling section of the first ODH reactor, thereby cooling the first reaction mixture on the tube side in the third cooling section, wherein the third cooling section is operationally downstream of the second cooling section and is separated from the second cooling section by a second flow barrier on the shell side. The method may include injecting oxygen into the first effluent. In implementations, the tube side in the third cooling section (of the first ODH reactor) does not include catalyst. The method may include heating water with at least one of the first coolant discharged from the first cooling section, the second coolant discharged from the second cooling section, or the first effluent, thereby facilitating generation of steam from the water. In implementations, the heating of the water (e.g., boiler feedwater) facilitating generation of steam from the water involves the heating the water vaporizing the water into steam or pre-heating the water for vaporization of the water into steam in a steam drum. The method may include heating the steam with the first coolant from the shell side of the first cooling section or with the second coolant from the shell side of the second cooling section, or both, thereby superheating the steam.

The method may include dehydrogenating, via ODH catalyst on a tube side of the second ODH reactor, ethane to ethylene in the second reaction mixture flowing through the tube side of the second ODH reactor, wherein the second ODH reactor has a third cooling section and a fourth cooling section. The second ODH reactor may be a multi-tubular fixed bed reactor having the tube side and the shell side, and the second reaction mixture flows through the tube side of the second ODH reactor. The method may include discharging the second reaction mixture from the second ODH reactor as a second effluent. The method may include flowing a third coolant through the shell side of the second ODH reactor in the third cooling section, thereby maintaining the ODH catalyst in the third cooling section at a third temperature; and flowing a fourth coolant through the shell side in the fourth cooling section, thereby maintaining the ODH catalyst in the fourth cooling section at a fourth temperature, wherein the fourth temperature is greater than the third temperature. The method may include maintaining temperature increase of the third coolant through the third cooling section below a third threshold; and maintaining temperature increase of the fourth coolant through the second cooling section below a fourth threshold. The method may include heating water with at least one of the first coolant discharged from the first cooling section, the second coolant discharged from the second cooling section, the third coolant discharged from the third cooling section, the fourth coolant discharged from the fourth cooling section, the first effluent, or the second effluent, wherein heating the water vaporizes the water into steam or pre-heats the water for vaporization of the water into steam in a steam drum.

Yet another embodiment is an ODH reactor system including a first ODH reactor (a multi-tubular fixed bed reactor) having a first cooling section and a second cooling section separated by a flow barrier on a first shell side. The second cooling section is operationally downstream of the first cooling section. The first ODH reactor includes a first tube side having ODH catalyst to receive feed including ethane, oxygen, and steam to dehydrogenate ethane into ethylene in a first reaction mixture and discharge a first effluent including ethylene, acetic acid, water, carbon dioxide, carbon monoxide, and unreacted ethane through a first-effluent discharge conduit to a second ODH reactor. The steam in the feed may act as a diluent to place the feed outside of flammability limits. The first ODH reactor includes the first shell side to receive a first coolant into the first cooling section to maintain temperature of the ODH catalyst in the first cooling section at a first temperature and receive a second coolant into the second cooling section to maintain temperature of the ODH catalyst in the second cooling section at a second temperature, wherein the first temperature is lower than the second temperature. The ODH reactor system includes the second ODH reactor (a multi-tubular fixed bed reactor) having a third cooling section and a fourth cooling section separated by a flow barrier on a second shell side. The fourth cooling section is operationally downstream of the third cooling section. The second ODH reactor includes a second tube side having ODH catalyst to receive the first effluent to dehydrogenate ethane into ethylene in a second reaction mixture and discharge a second effluent through a second-effluent discharge conduit. The second ODH reactor includes the second shell side to receive a third coolant into the third cooling section to maintain temperature of the ODH catalyst in the third cooling section at a third temperature and receive a fourth coolant into the fourth cooling section to maintain temperature of the ODH catalyst in the fourth cooling section at a fourth temperature, wherein the third temperature is lower than the fourth temperature. In implementations, the first temperature and the third temperature each are in a range of 300° C. to 450° C., and the second temperature and the fourth temperature each are in a range of 350° C. to 500° C.

The ODH reactor system may include a first-coolant supply system having a pump to provide the first coolant to the first cooling section and maintain temperature increase of the first coolant through the first cooling section to below a first threshold; a second-coolant supply system comprising a pump to provide the second coolant to the second cooling section and maintain temperature increase of the second coolant through the second cooling section to below a second threshold; a third-coolant supply system comprising a pump to provide the third coolant to the first cooling section and maintain temperature increase of the third coolant through the third cooling section to below a third threshold; and a fourth-coolant supply system comprising a pump to provide the fourth coolant to the fourth cooling section and maintain temperature increase of the fourth coolant through the fourth cooling section to below a second threshold. In implementations, the first threshold and the second threshold are in a range of 2° C. to 8° C. In implementations, the first coolant and the second coolant each are or include molten salt.

The ODH reactor system may include an oxygen supply conduit to inject oxygen into the first effluent flowing through the first-effluent discharge conduit. The ODH reactor system may include an injection-water conduit to inject liquid water into the first effluent flowing through the first-effluent discharge conduit to cool the first effluent. The ODH reactor system may include a heat exchanger disposed along the first-effluent discharge conduit to cool the first effluent with water, thereby heating the water for generating steam from the water. The first ODH reactor may have a fifth cooling section operationally downstream of second cooling section to cool the first reaction mixture flowing through the first tube side in the fifth cooling section, wherein the fifth cooling section and the second cooling section are separated by a second flow barrier on the first shell side. In implementations, the first tube side in the fifth cooling section does not have ODH catalyst or other catalyst.

The ODH reactor system may include a heat exchanger to heat water with the first coolant discharged from the first cooling section for generating steam from the water. The ODH reactor system may have a superheater heat exchanger to heat the steam with the first coolant from the first shell side or with the second coolant from the first shell side, or both, to superheat the steam. The ODH reactor system may include a heat exchanger to heat water with the second coolant discharged from the second cooling section for generating steam from the water. The ODH reactor system may have a heat exchanger to heat water with the third coolant discharged from the third cooling section for generating steam from the water. The ODH reactor system may have a heat exchanger to heat water with the fourth coolant discharged from the fourth cooling section for generating steam from the water. The ODH reactor system may have a heat exchanger disposed along the second-effluent discharge to heat water with the second effluent for generating steam from the water. The first ODH reactor may be configured to generate acetic acid from ethane in the first reaction mixture flowing through the first tube side. The second ODH reactor is configured to generate acetic acid from ethane in the second reaction mixture flowing through the second tube side.

### EXAMPLES

Examples (for Configurations 1-13) encompass reactions in ODH reactors occurring at temperature between 300-450° C. with low-temperature ODH catalyst (MoVNbTeOx previously discussed) to produce mainly ethylene with high selectivity. To stay outside of flammability envelope of ethane-oxygen mixture, a diluent is employed. Vaporized water can be utilized as the diluent to achieve staying outside of the flammability envelope. Based on pressure and temperature of mixed feed to reactor, the target oxygen concentration can differ. The present disclosure includes reactor design and set-up configurations with different stages and different reactor cooling schemes presented for comparison. In the configurations, HP/VHP steam is generated via cooling down the hot coolant (hot molten salt(s)) that discharge from different reactor sections. The comparison numbers (values) presented below are for the sake of example to show the relative advantages or disadvantages of one configuration over another one. Configurations 1-13 as the several configurations of reactor system and reactor design are summarized below and in view of the tabulated data (see Tables 2-4). Configuration 2 is selected as the base case in the comparison.

Reactor design was considered via gPROMS® platform software (gPROMS ProcessBuilder version 1.3.1) by Siemens Process Systems Engineering (PSE). Process simulations were performed with Aspen Plus® V10. The SR-POLAR equation of state was utilized for the simulations. Aspen Plus© software is available from Aspen Technology, Inc. having headquarters in Bedford, Massachusetts, USA.

Configuration 1 (an implementation of FIG. 3) is 1-stage reactor with 1 catalyst cooling section, 1-inch tubes, and <5° C. temperature rise in the coolant (salt). Includes TLE after reactor for HP/VHP steam generation or BFW pre-heating. This can be characterized as the simplest configuration to build a reactor; however, it requires the highest dilution possible due to having only one cooling section. It is relatively big reactor per unit of ethylene production. The reactor requires about 65% more tubes with about 35% longer tubes than Configuration 2 (baseline), which means about 125% more catalyst. This translates to longer residence time or smaller GHSV compared to Configuration 2. Selectivity of ethylene and AA are less than Configuration 2 while the CO/CO₂ selectivity are much higher. This means more molten salt to cool the reactor and more steam generation. Due to high dilution requirement and poorer performance compared to Configuration 2, the CO₂ intensity (mass ratio) (e.g., ton CO₂ emission per ton ethylene production) of the ODH plant (entire plant) is about 50% higher than Configuration 2.

Configuration 2 (an implementation of FIG. 4) is 1-stage reactor with 2 catalyst cooling sections, 1-inch tubes and <5° C. temperature rise in the molten salt, and TLE after reactor for HP/VHP steam generation or BFW pre-heating. Again, Configuration 2 is arbitrarily the base case in the comparison of Configurations 1-13. Configuration 2 generally has much better performance than Configuration 1, as described above.

Configuration 3 (an implementation of FIG. 4) is 1-stage reactor with 2 catalyst cooling sections, 1-inch tubes and about 15° C. temperature rise in the coolant (molten salt) across each of the two cooling sections, and TLE after reactor for HP/VHP steam generation or BFW pre-heating. Thus, the basis of Configuration 3 is same as Configuration 2, except the temperature increase of the salt is 15° C. instead of <5° C. The results of Configuration 3 show that the increased rise in the temperature of the coolant of the reactor (here molten salt) should generally be limited. The more temperature rise means less molten salt circulation resulting in reduced heat transfer coefficient (HTC). This would result in around 9% less ethylene and AA selectivity and 9% more CO/CO₂ selectivity compared to Configuration 2. To achieve the same amount of ethylene production, the number of tubes would be more than twice, and the length of tubes about 140% longer, which means about 420% more catalyst than in Configuration 2. This can be translated to longer residence time or smaller GHSV compared to Configuration 2. Due to high dilution requirement and poorer performance compared to Configuration 2, the CO₂ intensity of entire plant is about 40% higher than Configuration 2.

Configuration 4 (an implementation of FIG. 4) with 1-stage reactor with 2 catalyst cooling sections, 1.25-inch tubes, and <5° C. temperature rise in the molten salt, and TLE after reactor for HP/VHP steam generation or BFW pre-heating. The basis is the same as Configuration 2, except that 1.25-inch tubes are employed instead of 1-inch tubes. This shows that although larger tubes can be employed for reactor design, the heat removal and the performance are not as effective as smaller tubes as in Configuration 2. The larger tubes have less HTC inside the tubes and poorer heat removal. This would result in slightly less ethylene and AA selectivity, and slightly higher CO/CO₂ selectivity, compared to Configuration 2. To achieve the same amount of ethylene production, the number of tubes should be 20% less (easier to build), and the length of tubes is about 35% longer with about 65% more catalyst than Configuration 2. This can be translated to longer residence time or smaller GHSV compared to Configuration 2. Due to slightly higher dilution requirement and slightly poorer performance compared to Configuration 2, the CO₂ intensity of the ODH plant is about 5% higher than Configuration 2, which is very comparable. Nevertheless, less catalyst quantity makes the Configuration 2 more attractive.

Configuration 5 (an implementation of FIG. 4) with 1-stage reactor with 2 catalyst cooling sections, 1.5-inch tubes and <5° C. temperature rise in the molten salt and TLE after reactor for HP/VHP steam generation or BFW pre-heating. This shows that although lager tubes can be employed for reactor design, the heat removal and the performance are not as effective as with smaller tubes (Configurations 2 and 4). The larger tubes have less HTC inside the tubes and poorer heat removal. This would result in about 5% less ethylene and AA selectivity and 5% more CO/CO₂ selectivity compared to Configuration 2. To achieve the same amount of ethylene production, the number of tubes should be 20% less (easier to build), and the length of tubes is almost twice which means about 265% more catalyst than in Configuration 2. This can be translated to longer residence time or smaller GHSV compared to Configuration 2. Due to high dilution requirement and poorer performance compared to Configuration 2, the CO₂ intensity of ODH plant is about 25% higher than Configuration 2.

Configuration 6 (an implementation of FIG. 5) is 1-stage reactor with 2 catalyst cooling sections and 1 non-catalyst cooling section for BFW heating or V/HP steam generation, 1-inch tubes, and <5° C. temperature rise in the molten salt. This shows that instead of TLE after reactor, added to end portion of the reactor is another cooling section with molten salt circulation, and the hot molten salt utilized to pre-heat boiler feed water (BFW) or generate HP/VHP steam. This adds to the length of the reactor for this third non-catalyst cooling section and generally does not impact other aspects of reactor design or performance compared to Configuration 2.

Configuration 7 (an implementation of FIG. 6) is 1-stage reactor with 3 catalyst cooling sections, 1-inch tubes, <5° C. temperature rise in the molten salt, and TLE after reactor for HP/VHP steam generation or BFW pre-heating. This shows that the addition of extra cooling sections would generally improve the reactor performance, as the reaction can be carried out in a generally more temperature-controlled manner. This would result in slightly higher ethylene selectivity at the cost of less AA/CO/CO₂ selectivity compared to Configuration 2. To achieve the same amount of ethylene production, the number of tubes should be 10% less and 10% shorter, which means about 20% less catalyst than in Configuration 2. This can be translated to shorter residence time or larger GHSV compared to Configuration 2. Due to less dilution requirement and better performance compared to Configuration 2, the CO₂ intensity of the ODH plant is slightly lower than in Configuration 2. Configuration 7 illustrates that the number of catalyst cooling sections can be extended to “n”; however, this would add to complexity of reactor design and fabrication.

Configuration 8 (an implementation of FIG. 7) is 2-stage reactor system with 2 catalyst cooling sections in each stage, 1-inch tubes, <5° C. temperature rise in the molten salt, an inter-stage water quench, inter-stage oxygen addition, and TLE after 2nd reactor for HP/VHP steam generation or BFW pre-heating. Again, this configuration is a two-stage reactor system. All ethane, a portion of oxygen, and water diluent enters the first reactor with 2 catalyst cooling sections. The first reactor converts almost all of the oxygen that enters (fed to) the first reactor. The effluent from first reactor is quenched with liquid water to reduce the temperature of the gas (effluent). Then oxygen is added. The addition of quench water would prevent a sudden autoignition of gas components (ethylene, ethane, AA, CO) when oxygen is added. A main idea for 2-stage reactor is to split the oxygen between two stages. This would reduce the oxygen in the first stage and consequently the dilution water demand for the first stage and the entire reactor system. Because in the second stage, the water that has been added to first stage plus the generated water are still in the mixture, so there is no need for extra dilution.

This 2-stage reactor system with inter-stage water quenching and inter-stage addition would result in slightly lower ethylene selectivity; however, it would increase AA selectivity by over 2% and reduce the CO/CO₂ selectivity as compared to Configuration 2. To achieve the same amount of ethylene production, the first stage reactor is much smaller than the second stage reactor. For the first stage, the number of tubes should be 65% less and 60% shorter than in Configuration 2. On the other hand, for the second stage, the number of tubes should be 30% more and 15% shorter than in the single reactor of Configuration 2. The second stage reactor is relatively bigger but comparable to one-single stage reactor (Configuration 2). The overall catalyst is about 25% more for 2-stage of reactor compared to Configuration 2 with 1-stage reactor. The residence time for first stage is very short while the second stage has a comparable residence time to the reactor in Configuration 2. Due to much less dilution requirement and slightly better performance compared to Configuration 2, the CO₂ intensity of the ODH plant is 40% lower than Configuration 2.

Configuration 9 (an implementation of FIG. 8) is 2-stage reactor system with 2 catalyst cooling sections in each stage, 1-inch tubes, <5° C. temperature rise in the molten salt, 1 non-catalyst cooling section at the end of the 1 st stage, oxygen at the inter-stage, and TLE after second reactor for HP/VHP steam generation or BFW pre-heating. Similar to Configuration 8 but instead of quenching the effluent from 1 st stage reactor with quench water, an extra cooling section that is a non-catalyst cooling section is added to the end of the first stage reactor. This means that the first stage has two catalyst-cooling sections with molten salt circulation and one non-catalyst cooling section with molten salt circulation. This extra section would add to complexity of 1 st reactor design and fabrication but may significantly improve heat recovery from the first stage reactor effluent. In Configuration 8, the quench water is evaporated, and thus no heat recovery is carried out. In contrast, the hot molten salt from this third cooling section in Configuration 9 pre-heats BFW or generates HP/VHP steam.

Configuration 9 has slightly lower ethylene selectivity than Configuration 8; however, it would slightly increase AA selectivity and reduce the CO/CO₂ selectivity as compared to Configuration 8. To achieve the same amount of ethylene production, the first stage reactor is much smaller than the second stage reactor. For the first stage, the number of tubes should be 60% less and 40% shorter than the sole reactor in Configuration 2. However, it is larger than first stage reactor of Configuration 8. On the other hand, for the second stage, the number of tubes should be 20% more and 15% shorter than in the sole reactor in Configuration 2. The second stage reactor is relatively comparable to one-single stage reactor (Configuration 2). However, the second stage of Configuration 9 is smaller than second stage reactor of Configuration 8. The overall catalyst is about 15% more for second reactor (2-stage reactor) compared to sole reactor in Configuration 2 (1-stage reactor). The residence time for first stage is very short while the second stage has a comparable residence time to Configuration 2. Due to much less dilution requirement and significant heat recovery from effluent of first stage reactor, and slightly better performance compared to Configuration 2, the CO₂ intensity of entire plant is 55% lower than with Configuration 2 and nearly 10% less than Configuration 8.

Configuration 10 (an implementation of FIG. 9) is 2-stage reactor system with 2 catalyst cooling sections in each stage, 1-inch tubes, <5° C. temperature rise in the molten salt, HEX quenching at inter-stage, oxygen addition at the inter-stage, and TLE after second reactor for HP/VHP steam generation or BFW pre-heating. Similar to Configuration 10 but instead of third cooling section in first stage reactor, a HEX is employed between the two stages. This HEX is similar to TLE after the entire reactor system and can be used for either BFW pre-heating or HP/VHP steam generation. There is generally no difference between the performance of Configuration 10 and Configuration 9.

Configuration 11 (an implementation of FIG. 10) is 3-stage reactor system with 2 catalyst cooling sections in each stage, 1-inch tubes, <5° C. temperature rise in the molten salt, water quench at inter-stage, oxygen addition at the inter-stage, and TLE after third reactor for HP/VHP steam generation or BFW pre-heating. This is similar to Configuration 8 but with a third reactor. All ethane, a portion of oxygen, and water diluent enter the first reactor (with 2 catalyst cooling sections) that converts almost all of the oxygen. The effluent from first reactor is quenched with water liquid to reduce the temperature of the gas (effluent). Then oxygen is added at the inter-stage between the first reactor and the second reactor. The addition of quench water would prevent a sudden autoignition of gas components (ethylene, ethane, AA, CO) when oxygen is added. This effluent goes through the second stage with similar approach as first stage. The effluent from second stage is again quenched using quench water and then oxygen is added, and it goes to third stage. A main idea for a third reactor (3-stage reactor) is to split the oxygen between three stages. This would reduce the oxygen in the first stage and consequently the dilution water demand for the first stage and the entire reactor system. Because in the second and third stages, the water that has been added to first stage is still in the mixture, so there is no need for extra dilution.

This 3-stage reactor system with water quenching and oxygen inter-staging would result in lower ethylene selectivity; however, it would increase AA selectivity by about 4% and reduce the CO/CO₂ selectivity as compared to Configuration 2. It shows the same trend from 2-stage (Configuration 8) to 3-stage. The AA selectivity is improved and the ethylene, CO/CO₂ selectivity's are declining. To achieve the same amount of ethylene production, the first stage reactor is much smaller than the second and third stage reactors. The second stage is also much smaller than third stage. For the first stage, the number of tubes should be 80% less and 70% shorter than Configuration 2. For second stage, the number of tubes should be 40% less and 55% shorter than Configuration 2. On the other hand, for the third stage, the number of tubes should be 60% more but about 30% shorter than Configuration 2. The overall catalyst is about 40% more for third reactor (3-stage reactor) compared to Configuration. 2 and even is more than Configuration 8. The residence time for first and second stage is very short while the third stage has a comparable residence time to Configuration 2 and second stage of Configuration 8. Due to much less dilution requirement and slightly better performance compared to Configuration 2, the CO₂ intensity of entire plant is 50% lower than Configuration 2, which is 10% better (lower) than Configuration 8. This shows that adding stages would improve the overall performance of the ODH plant. However, the move from 1 stage to 2 stage reduced the CO₂ intensity by 40% and the incremental from 2-stage to 3-stage is only 10%, which may make the 2-stage is more attractive. Adding another stage would add the cost to capital and operating of the plant and complexity of reactor design and fabrication.

Configuration 12 (an implementation of FIG. 11) is 3-stage reactor system with 2 catalyst cooling sections in each stage, 1-inch tubes, <5° C. temperature rise of the molten salt, 1 non-catalyst cooling section at the end of the 1 st and 2nd stages, oxygen injection at the inter-stage, and TLE after third reactor for HP/VHP steam generation or BFW pre-heating. Similar to Configuration 11 but instead of quenching the effluent from 1 st and 2nd stage reactors with quench water, an extra non-catalyst cooling section is added to the 1st and 2nd stage reactors. This means that the 1st and 2nd stages have two catalyst cooling sections with the molten salt circulation and one non-catalyst cooling section with the molten salt circulation. This extra section would add to complexity of the 1st and 2nd reactors design and fabrication but will significantly improve heat recovery from the 1 st and 2nd stage reactor effluent. In Configuration 11, the quench water is evaporated, and no heat recovery is carried out. In contrast, for Configuration 12, the hot molten salt from the third cooling section is used to pre-heat BFW or V/HP steam generation.

Furthermore, Configuration 12 has slightly lower ethylene selectivity Configurations 2, 9, and 11; however, it would slightly increase AA selectivity and reduce the CO/CO₂ selectivity as compared to Configurations 2, 9, and 11. To achieve the same amount of ethylene production, the first stage reactor is much smaller than the second and third stage reactors. In addition, the 2nd stage is much smaller than the 3rd stage. For the first stage, the number of tubes should be 75% less and 55% shorter than in Configuration 2. For second stage, the number of tubes should be 25% less and 45% shorter than in Configuration 2. On the other hand, for the third stage, the number of tubes should be 40% more but about 35% shorter than in Configuration 2. The overall catalyst is around 15% more for the 3-stage reactor (third reactor) compared to Configuration 2 but similar to the 2-stage reactor (second reactor) of Configuration 9. The residence time for the 1 st and 2nd stages is very short while the third stage has a comparable residence time to Configuration 2. Due to much less dilution requirement and higher heat recovery from effluent of the 1st and 2nd stage reactors, and slightly better performance compared to Configurations 2 and 9, the CO₂ intensity of the ODH plant is 70% lower than Configuration 2, almost 15% less than Configuration 9, and 20% lower than Configuration 11. Thus, adding extra stages would improve the overall performance of the ODH plant with respect to CO₂ intensity.

Configuration 13 (an implementation of FIG. 12) is 3-stage reactor system with 2 catalyst cooling sections in each stage, 1-inch tubes, <5° C. temperature rise in the molten salt, HEX quenching at inter-stage, oxygen addition at the inter-stage, and TLE after third reactor for HP/VHP steam generation or BFW pre-heating. Similar to Configuration 12 but instead of third cooling section in 1 st and 2nd stage reactors, a HEX is employed between the stages (see FIG. 12). These HEXs are similar to TLE after the entire reactor system and can be used for either BFW pre-heating or HP/VHP steam generation. There is generally no difference between the performance of Configuration 13 and Configuration 12.

Table 2 is data for Configurations 1-7, which are single-reactor systems having a single ODH reactor. The ΔT coolant is the temperature increase (temperature rise) of coolant (molten salt) through each cooling section. The number (#) of tubes, tube length, active catalyst (ODH catalyst), space time yield (STY), and total flow rate are assigned 100% for Configuration 2 as base case, and with the remaining configurations in percent relative to that 100% of Configuration 2. The total mass flow rate is total mass flow rate (mass per time) of the feed (ethane, oxygen, and dilution steam) to the ODH reactor. A TLE is not employed for Configuration 6 because the third cooling section in Configuration 6 is a non-catalyst cooling section. This non-catalyst cooling section cools the reaction mixture with coolant (molten salt) before the reaction mixture discharges as the effluent, and in which the cooling can thus be labeled as a salt quench. The Δ symbol for ethane conversion means the value listed is the absolute percent difference with the ethane conversion of Configuration 2. Likewise, the A symbol for the three rows of selectivity ethane conversion means the value listed is the absolute percent difference with the given conversion of Configuration 2. The O₂/ethane mass ratio and the water/ethane mass ratio are for the feed to the ODH reactor and in which the water is dilution steam. The amount (mass per time) of ethane feed is the same in all Configurations 1-7. Lastly, while tube diameter in various applications may be outside diameter (OD), inside diameter (ID), or nominal diameter, the specified numerical value for diameter in Table 2 is OD for the simulation. In these Examples for the simulation, a 1-inch tube is OD is 1 inch, tube wall thickness is 0.083 inch, and ID is 0.834 inch; a 1.25-inch tube as OD is 1.25 inch, tube wall thickness is 0.120 inch, and ID is 1.01 inch; and a 1.5-inch tube as GD is 1.5 inch, tube wall thickness is 0.134 inch, and ID=1.232 inch.

TABLE 2
Data for Configurations 1-7

| Configuration | 1 | 2¹ | 3 | 4 | 5 | 6 | 7 |
|---|---|---|---|---|---|---|---|
| # Cooling Sections | 1 | 2 | 2 | 2 | 2 | 3³ | 3⁴ |
| ΔT Coolant | <5° C. | <5° C. | 15° C. | <5° C. | <5° C. | <5° C. | <5° C. |
| TLE | Yes | Yes | Yes | Yes | Yes | No | Yes |
| Tube Size (OD) | 1″ | 1″ | 1″ | 1.25″ | 1.5″ | 1″ | 1″ |
| Comparison to | 2 | — | 2 | 2 | 2/4 | 2 | 1/2 |
| # Tubes | 163% | 100% | 206% | 82% | 79% | 100% | 88% |
| Tube Length | 135% | 100% | 241% | 134% | 201% | 116% | 91% |
| Active Catalyst | 223% | 100% | 515% | 166% | 366% | 100% | 80% |
| GHSV (1/hr) | 4675 | 7057 | 1880 | 4541 | 2387 | 7057 | 8556 |
| STY | 45% | 100% | 19% | 60% | 27% | 100% | 125% |
| Linear Velocity (cm/s) | 323 | 365 | 236 | 311 | 248 | 248 | 389 |
| Ethane Conversion Δ² | 1.8% | 0.0% | 6.2% | 0.9% | 3.1% | 0.0% | −0.9% |
| Ethylene Selectivity Δ² | −2.0% | 0.0% | −6.5% | −1.1% | −3.3% | 0.0% | 0.9% |
| AA Selectivity Δ² | −2.9% | 0.0% | −2.1% | −0.3% | −1.6% | 0.0% | −0.6% |
| CO/CO₂ Selectivity Δ² | 4.8% | 0.0% | 8.5% | 1.3% | 4.8% | 0.0% | −0.4% |
| O₂/Ethane Mass Ratio | 0.6 | 0.6 | 0.8 | 0.6 | 0.5 | 0.2 | 0.2 |
| Water/Ethane Mass | 3.7 | 2.2 | 3.3 | 2.4 | 2.8 | 2.2 | 2.1 |
| Ratio | | | | | | | |
| Total Mass Flow Rate | 141% | 100% | 133% | 105% | 119% | 100% | 97% |

¹All the configurations are compared to Configuration 2.
²Δ vs Configuration 2 (e.g., ethane conversion Δ for Configuration 1 = ethane conversion configuration 1 minus ethane conversion Configuration 2).
³The 3rd section is in lieu of TLE and a non-catalyst molten salt cooling section.
⁴The 3rd section is the 3rd catalyst molten salt cooling section.

Table 3 is data for Configurations 8-13. As discussed, Configurations 8-13 are multi-reactor systems having more than one ODH reactor (stage). All Configurations 8-13 have the same the amount (mass per time) of ethane feed to the ODH reactor system (in particular, to the first ODH reactor). All Configurations 8-13 have 1″ tubes in the ODH reactors (with 1″ being an outside diameter (GD) of the tubes for the simulation), a TLE after the final ODH reactor, and a temperature increase of <5° C. of the coolant molten salt through each cooling section. The number (#) of cooling sections is given for each stage. For instance, the 3/2 listed for Configuration 9 means three cooling sections in the first reactor (first stage) and two cooling sections in the second reactor (second stage). The number (#) of tubes, tube length, active catalyst (ODH catalyst), space time yield (STY), total flow rate are assigned 100% for the sole ODH reactor in Configuration 2 as base case, and with the corresponding values for the Configurations 8-13 ODH reactors in percent relative to that 100% of Configuration 2. The total mass flow rate is total flow (mass per time) of the feed (ethane, oxygen, and dilution steam) to the first ODH reactor, the oxygen injected inter-stage, and any water that is injected at the inter-stages (for a water quench configuration 8 and 11). As with Table 2, the Δ symbol for ethane conversion (of the multi-reactor system) means the value listed is the absolute percentage difference with the ethane conversion of the single-reactor system of Configuration 2. Likewise, the Δ symbol for selectivity means the value listed is the absolute percentage difference with the given conversion of Configuration 2. The O₂/ethane mass ratio is the mass ratio of the total oxygen (feed to first ODH reactor plus inter-stage injected) to the ethane fed to the first ODH reactor. The water/ethane mass ratio is the mass ratio of the water (dilution steam) in the feed to the first ODH reactor (and any inter-stage injected liquid water for the purpose of water quench in Configurations 8 and 11) to the ethane fed to the first ODH reactor.

TABLE 3
Data for Configurations 8-13

| Configuration | 2¹ | 8 | 9 | 10 | 11 | 12 | 13 |
|---|---|---|---|---|---|---|---|
| # Stages | 1 | 2 | 2 | 2 | 3 | 3 | 3 |
| # Cooling Sections | 2 | 2/2 | 3/2 | 2/2 | 2/2/2 | 3/3/2 | 2/2/2 |
| Inter-Stage Quench | NA | Water | Salt | HEX | Water | Salt | HEX |
| Comparison to | — | 2 | 2/8 | 2/8/9 | 2/8 | 2/9/11 | 2/10/12/13 |
| # Tubes 1^{st} Stage² | 100% | 34% | 43% | 43% | 20% | 25% | 25% |
| Tube Length 1^{st} Stage² | 100% | 60% | 60% | 44% | 30% | 44% | 44% |
| # Tubes 2^{nd} Stage² | NA | 118% | 118% | 118% | 60% | 73% | 73% |
| Tube Length 2^{nd} Stage² | NA | 84% | 84% | 84% | 45% | 54% | 54% |
| # Tubes 3^{rd} Stage² | NA | | | | 161% | 139% | 139% |
| Tube Length 3^{rd} Stage² | NA | | | | 68% | 63% | 63% |
| Total Active Catalyst in | 100% | 125% | 116% | 116% | 137% | 116% | 116% |
| All Stages | | | | | | | |
| GHSV 1^{st} Stage (1/hr) | 7057 | 23,170 | 19,099 | 19,099 | 38,345 | 40,189 | 40,189 |
| GHSV 2^{nd} Stage (1/hr) | NA | 5,092 | 4,622 | 4,622 | 15,295 | 19,398 | 19,398 |
| GHSV 3^{rd} Stage (1/hr) | NA | | | | 4,988 | 6,798 | 6,798 |
| STY 1^{st} Stage | 100% | 422% | 337% | 337% | 638% | 700% | 700% |
| STY 2^{nd} Stage | NA | 41% | 42% | 42% | 131% | 123% | 123% |
| STY 3^{rd} Stage | NA | | | | 33% | 33% | 33% |
| STY Reactor System | 100% | 80% | 86% | 86% | 73% | 87% | 87% |
| Linear Velocity | 365 | 439 | 396 | 396 | 490 | 471 | 471 |
| 1^{st} Stage (cm/s) | | | | | | | |
| Linear Velocity | NA | 246 | 250 | 250 | 304 | 259 | 259 |
| 2^{nd} Stage (cm/s) | | | | | | | |
| Linear Velocity | NA | | | | 187 | 185 | 185 |
| 3^{rd} Stage (cm/s) | | | | | | | |
| ΔC2 Conversion³ | 0% | 0.8% | 1.0% | 1.0% | 1.8% | 1.6% | 1.6% |
| ΔC2 = Selectivity³ | 0% | −0.9% | −1.1% | −1.1% | −2.0% | −1.8% | −1.8% |
| ΔAA Selectivity³ | 0% | 2.2% | 2.7% | 2.7% | 3.6% | 3.9% | 3.9% |
| Δ(CO/CO₂) Selectivity³ | 0% | −1.3% | −1.7% | −1.7% | −1.7% | −2.2% | −2.2% |
| O₂/C2 Mass Ratio | 0.6 | 0.2 | 0.2 | 0.2 | 0.1 | 0.2 | 0.2 |
| Water/C2 Mass Ratio | 2.2 | 0.6 | 0.7 | 0.7 | 0.2 | 0.4 | 0.4 |
| Total Flow Rate | 100% | 48% | 53% | 53% | 34% | 40% | 40% |

¹All the configurations are compared to Configuration 2.
²# of tubes and Tube length for every stage (more than 1 stage reactor is compared to Configuration 2 with one stage reactor).
³Δ vs Configuration 2 (e.g., ΔC2 conversion for Configuration 8 = overall C2 conversion Configuration 8 minus C2 conversion Configuration 2).

Table 4 gives results of Configurations 1-13. The first row is the Configuration number. Configurations 1-7 are ODH reactor systems having one ODH reactor (single-reactor system). Configurations 8-13 are ODH reactor systems having more than one ODH reactor (multi-reactor system). In particular, Configurations 8-10 have two ODH reactors in series (two-stage reactor system) and Configurations 11-13 have three ODH reactors in series (three-stage reactor system).

The second row in Table 4 is the major duty (heat) demand of the ODH plant, which is a combination of (1) heat demand of dilution steam generation for the reactor feed and (2) solvent recovery in the acetic acid unit. More energy may be consumed for solvent recovery in the AA unit due to presence of more water. The more the dilution steam, the more the cooling demand to condense the water/AA after the reactor system. More dilution steam also gives more water in AA to the AA unit. Therefore, more the solvent is utilized to separate AA from the water and thus the more heat for reboiler of the solvent recovery tower, and more cooling demand for the condenser of the solvent recovery tower.

The third row is the major cooling demand in the ODH plant, which is a combination of (1) cooling demand for condensing (including condensing water) in the reactor effluent and (2) cooling demand for solvent recovery in the acetic acid unit. The fourth row in Table 2 is the reactor HP/VHP steam generation. The fifth row is the mass ratio (e.g., kg/kg) of LP/MP steam demand for the ODH plant to HP/VHP steam generated at the reactor system. The sixth row is CO₂ intensity of the ODH plant (emitted and non-emitted), which is generated CO₂ emissions per ethylene production.

In Table 4, the duty demand, cooling demand, steam generation, and CO₂ intensity are assigned 100% for Configuration 2 as base case, and with the remaining configurations having a percentage relative to the 100% of Configuration 2. The duty demand and cooling demand (and thus CO₂ intensity) are significantly reduced for a two-stage reactor system compared to a one-stage reactor system because less oxygen (and thus less dilution steam) is fed to the first reactor in the two-reactor system.

TABLE 4
Results of Configurations 1-13

| Conf | 1 | 2 | 3 | 4 | 5 | 6 | 7 | 8 | 9 | 10 | 11 | 12 | 13 |
|---|---|---|---|---|---|---|---|---|---|---|---|---|---|
| Duty¹ | 141% | 100% | 129% | 104% | 115% | 100% | 95% | 51% | 51% | 51% | 140% | 37% | 37% |
| Cool² | 137% | 100% | 128% | 104% | 114% | 100% | 96% | 66% | 58% | 58% | 59% | 46% | 46% |
| Stm | 115% | 100% | 147% | 107% | 124% | 100% | 96% | 86% | 106% | 106% | 86% | 108% | 108% |
| Gen³ | | | | | | | | | | | | | |
| Stm | 3.3 | 2.7 | 2.4 | 2.6 | 2.5 | 2.7 | 2.7 | 1.6 | 1.3 | 1.3 | 1.3 | 1.0 | 1.0 |
| Ratio⁴ | | | | | | | | | | | | | |
| CO₂ | 151% | 100% | 141% | 105% | 122% | 100% | 97% | 58% | 47% | 47% | 50% | 31% | 31% |
| Intens⁵ | | | | | | | | | | | | | |

¹Overall Duty Demand (LP/MP Steam) of major duty demand areas (Dilution Steam Generation and Acetic Acid Solvent Recovery section) vs. Configuration 2.
²Overall Cooling Demand (Air Cooler or Cooling Water) of major cooling demand areas (Reactor Effluent Cooling & Acetic Acid Solvent Recovery sections) vs. Configuration 2.
³HP/VHP Steam Generation by Reactor System vs. Configuration 2.
⁴“LP/MP Steam Demand of ODH Plant” To “HP/VHP Steam Generated by Reactor System”.
⁵CO2 Intensity of ODH Plant (Ton CO2 Generated [emitted and non-emitted] per Ton Ethylene Production) vs. Configuration 2.

In general for the configurations, any combinations of water quenching, HEX quenching, or non-catalyst salt quenching for effluent of each stage of reactor can be employed. In addition, the ethane or water vapor can also be added at inter-stage. Moreover, the salt flow versus process flow inside tubes can be co-current or counter current (e.g., see FIGS. 4 and 4A). In configurations with more than one stage, each stage could be co-current or counter current. This means that for example, if the first stage is co-current, the second stage could be co-current or counter current, or if the first stage is counter current, the second stage could be co-current or counter current.

The following may be benefits of embodiments of the present techniques. A TLE after the sole or final reactor can help to recover more heat. An extra cooling section as a non-catalyst section at the end of each stage can also help to recover heat for HP/VHP steam generation.

Adding more catalyst cooling sections would improve the performance of the reactor to generate more ethylene and AA and less undesired CO/CO₂. It may help to control the temperature of reaction. In the first cooling section of a reactor, most of oxygen may be consumed and in the next sections of that reactor, the remainder of the oxygen may be consumed while maintaining good selectivity towards desired products.

Limiting the temperature rise in the salt to about 5° C. could improve the overall performance of the reactor in terms of generation of more desired products. The more temperature rise is allowed for salt in each cooling section generally means less molten salt circulation that would reduce the overall HTC on the molten salt side and result in poorer heat removal from the reaction inside the tubes.

The smaller the tubes gives better reactor performance in terms of desired products. It will improve the HTC inside the tubes (on the tube side) and thus improve (increase) heat removal from inside the tubes.

Adding extra stage (an extra reactor) to an ODH reactor system to give 2-stage system or 3-stage system (or n-stage stage system with n=3+) would help to split the oxygen between stages and reduce the dilution requirement for the reactor system (for the first stage) which would improve the entire plant efficiency. Less dilution steam means less energy consumption by the ODH plant. The generation of dilution steam requires energy. Downstream, the vapor water in the reactor-system effluent is condensed (along with AA) to the AA unit. This means energy consumed for condensation of water from reactor effluent and for the AA unit.

Water quenching can be employed as an effective technique to cool down the reactor effluent between stages before adding oxygen. HEX quenching or extra non-catalyst salt quenching section at the end of each stage could be more effective than water quenching due to heat recovery using molten salt or HEX quenching. This additional heat recovery compared to a water quench would improve the entire plant performance with respect to energy and CO₂ intensity.

A number of implementations have been described. Nevertheless, it will be understood that various modifications may be made without departing from the spirit and scope of the disclosure.

1. A method of operating an oxidative dehydrogenation (ODH) reactor system, the method comprising:
providing feed comprising ethane, oxygen, and diluent to a first ODH reactor, wherein the ODH reactor system comprises the first ODH reactor and a second ODH reactor each being a multi-tubular fixed bed reactor having a shell side and a tube side, wherein the first ODH reactor and the second ODH reactor each comprise a first cooling section and a second cooling section, and wherein the second ODH reactor is disposed in series operationally downstream of the first ODH reactor;
dehydrogenating ethane to ethylene via ODH catalyst in a reaction mixture flowing on the tube side of each of the first ODH reactor and the second ODH reactor;
flowing coolant through the shell side in the first cooling section and the second cooling section of each of the first ODH reactor and the second ODH reactor, thereby cooling the reaction mixture and the ODH catalyst on the tube side;
maintaining temperature increase of the coolant through each of the first cooling section and the second cooling section of each of the first ODH reactor and the second ODH reactor to below a threshold;
discharging a first effluent from the first ODH reactor to the second ODH reactor, the first effluent comprising ethylene, acetic acid, water, carbon dioxide, carbon monoxide, and unreacted ethane; and
injecting oxygen into the first effluent.

2. The method of claim 1, comprising cooling the first effluent upstream of injecting the oxygen into the first effluent or cooling the reaction mixture in a third cooling section in the first ODH reactor that discharges as the first effluent from the first ODH reactor, or a combination thereof, wherein the threshold is in a range of 2° C. to 8° C., wherein the coolant comprises molten salt, wherein the diluent comprises steam, and wherein the first ODH reactor and the second ODH reactor each comprise a flow barrier on the shell side separating the first cooling section and the second cooling section.

3. The method of claim 1, wherein cooling the reaction mixture comprises:
maintaining the reaction mixture on the tube side in the first cooling section of the first ODH reactor at a temperature lower than temperature of the reaction mixture on the tube side in the second cooling section of the first ODH reactor; and
maintaining the reaction mixture on the tube side in the first cooling section of the second ODH reactor at a temperature lower than temperature of the reaction mixture on the tube side in the second cooling section of the second ODH reactor.

4. The method of claim 3, wherein the temperature of the reaction mixture in the first cooling section of each of the first ODH reactor and the second ODH reactor is in a range of 300° C. to 450° C., and wherein the temperature of the reaction mixture in the second cooling section of each of the first ODH reactor and second ODH reactor is in a range of 350° C. to 500° C.

5. The method of claim 1, comprising heating water with coolant discharged from at least one of the first cooling section of the first ODH reactor, the second cooling section of the first ODH reactor, the first cooling section of the second ODH reactor, or the second cooling section of the second ODH reactor, wherein heating the water vaporizes the water into steam or pre-heats the water for vaporization of the water into steam in a steam drum.

6. The method of claim 1, comprising injecting liquid water into the first effluent, thereby vaporizing the liquid water via heat from the first effluent to cool the first effluent.

7. The method of claim 1, comprising cooling the first effluent by a heat exchanger with boiler feedwater as cooling medium, thereby vaporizing the boiler feedwater into steam or heating the boiler feedwater for vaporizing the boiler feedwater into steam in a steam drum.

8. The method of claim 1, wherein for the first ODH reactor and the second ODH reactor, the second cooling section is operationally downstream of the first cooling section in flow direction of the reaction mixture and is separated from the first cooling section by a flow barrier on the shell side.

9. The method of claim 8, comprising flowing coolant through the shell side in a third cooling section of the first ODH reactor, thereby cooling the reaction mixture on the tube side in the third cooling section, wherein the third cooling section is operationally downstream of the second cooling section of the first ODH reactor in the flow direction of the reaction mixture and is separated from the second cooling section by a second flow barrier on the shell side.

10. The method of claim 9, wherein the tube side in the third cooling section of the first ODH reactor flows does not comprise catalyst.

11. The method of claim 1, comprising:
discharging a second effluent from the second ODH reactor, the second effluent comprising ethylene, acetic acid, water, carbon dioxide, and carbon monoxide; and
generating steam via heat from at least one of the coolant discharged from the first ODH reactor, the coolant discharged from the second ODH reactor, the first effluent discharged from the first ODH reactor, or the second effluent discharged from the second ODH reactor.

12. The method of claim 11, comprising heating the steam with coolant from the first ODH reactor or the second ODH reactor, or both, thereby superheating the steam.

13. A method of operating an oxidative dehydrogenation (ODH) reactor system, the method comprising:
providing feed comprising ethane, oxygen, and diluent to a first ODH reactor comprising a tube side and a shell side to give a first reaction mixture flowing through the tube side;
dehydrogenating, via ODH catalyst on the tube side, ethane to ethylene in the first reaction mixture flowing through the tube side, wherein the first ODH reactor comprises a first cooling section and a second cooling section operationally downstream of the first cooling section;
flowing a first coolant through the shell side in the first cooling section, thereby maintaining the ODH catalyst in the first cooling section at a first temperature;
flowing a second coolant through the shell side in the second cooling section, thereby maintaining the ODH catalyst in the second cooling section at a second temperature; and
specifying the first temperature be less than the second temperature to favor ethylene selectivity in the first reaction mixture in the first cooling section.

14. The method of claim 13, comprising:
specifying temperature increase of the first coolant through the first cooling section be below a first threshold to favor ethylene selectivity in the first reaction mixture; and
specifying temperature increase of the second coolant through the second cooling section be below a second threshold to favor ethylene selectivity in the first reaction mixture.

15. The method of claim 13, comprising:
maintaining temperature increase of the first coolant through the first cooling section below a first threshold, wherein the first temperature is less than the second temperature; and
maintaining temperature increase of the second coolant through the second cooling section below a second threshold, wherein the feed comprises the diluent to maintain the feed outside of flammability limits, and wherein the diluent comprises steam.

16. The method of claim 13, comprising discharging the first reaction mixture from the tube side as a first effluent from the first ODH reactor to a second ODH reactor to give a second reaction mixture flowing through the second ODH reactor, wherein the first cooling section and the second cooling section are separated by a flow barrier on the shell side.

17. The method of claim 16, comprising:
cooling the first effluent by injecting liquid water into the first effluent or by a heat exchanger with boiler feedwater as cooling medium; and
injecting oxygen into the first effluent, wherein the first effluent as discharged from the first ODH reactor comprises ethylene, acetic acid, water, carbon dioxide, carbon monoxide, and unreacted ethane, wherein the second ODH reactor is a multi-tubular fixed bed reactor having a tube side and a shell side, and wherein the second reaction mixture flows through the tube side of the second ODH reactor.

18. The method of claim 16, comprising flowing third coolant through the shell side in a third cooling section of the first ODH reactor, thereby cooling the first reaction mixture on the tube side in the third cooling section, wherein the third cooling section is operationally downstream of the second cooling section and is separated from the second cooling section by a second flow barrier on the shell side.

19. The method of claim 18, comprising injecting oxygen into the first effluent, wherein the tube side in the third cooling section does not comprise catalyst.

20. The method of claim 16, comprising heating water with at least one of the first coolant discharged from the first cooling section, the second coolant discharged from the second cooling section, or the first effluent, thereby facilitating generation of steam from the water.

21. The method of claim 20, wherein heating the water facilitating generation of steam from the water comprises the heating of the water vaporizing the water into steam or pre-heating the water for vaporization of the water into steam in a steam drum, and wherein the water comprises boiler feedwater.

22. The method of claim 20, comprising heating the steam with the first coolant from the shell side of the first cooling section or with the second coolant from the shell side of the second cooling section, or both, thereby superheating the steam.

23. The method of claim 16, comprising:
dehydrogenating, via ODH catalyst on a tube side of the second ODH reactor, ethane to ethylene in the second reaction mixture flowing through the tube side of the second ODH reactor, wherein the second ODH reactor comprises a third cooling section and a fourth cooling section; and
discharging the second reaction mixture from the second ODH reactor as a second effluent.

24. The method of claim 23, comprising:
flowing a third coolant through a shell side of the second ODH reactor in the third cooling section, thereby maintaining the ODH catalyst in the third cooling section at a third temperature; and
flowing a fourth coolant through the shell side in the fourth cooling section, thereby maintaining the ODH catalyst in the fourth cooling section at a fourth temperature, wherein the fourth temperature is greater than the third temperature.

25. The method of claim 24, comprising:
maintaining temperature increase of the third coolant through the third cooling section below a third threshold; and
maintaining temperature increase of the fourth coolant through the second cooling section below a fourth threshold.

26. The method of claim 24, comprising heating water with at least one of the first coolant discharged from the first cooling section, the second coolant discharged from the second cooling section, the third coolant discharged from the third cooling section, the fourth coolant discharged from the fourth cooling section, the first effluent, or the second effluent, wherein heating the water vaporizes the water into steam or pre-heats the water for vaporization of the water into steam in a steam drum.

27. An oxidative dehydrogenation (ODH) reactor system, comprising:
a first ODH reactor comprising a first cooling section and a second cooling section separated by a flow barrier on a first shell side, the second cooling section operationally downstream of the first cooling section, wherein the first ODH reactor is a multi-tubular fixed bed reactor comprising:
a first tube side having ODH catalyst to receive feed comprising ethane, oxygen, and steam to dehydrogenate ethane into ethylene in a first reaction mixture and discharge a first effluent comprising ethylene, acetic acid, water, carbon dioxide, carbon monoxide, and unreacted ethane through a first-effluent discharge conduit to a second ODH reactor;
the first shell side to receive a first coolant into the first cooling section to maintain temperature of the ODH catalyst in the first cooling section at a first temperature and receive a second coolant into the second cooling section to maintain temperature of the ODH catalyst in the second cooling section at a second temperature, wherein the first temperature is lower than the second temperature; and
the second ODH reactor comprising a third cooling section and a fourth cooling section separated by a flow barrier on a second shell side, the fourth cooling section operationally downstream of the third cooling section, wherein the second ODH reactor is a multi-tubular fixed bed reactor comprising:
a second tube side having ODH catalyst to receive the first effluent to dehydrogenate ethane into ethylene in a second reaction mixture and discharge a second effluent through a second-effluent discharge conduit; and
the second shell side to receive a third coolant into the third cooling section to maintain temperature of the ODH catalyst in the third cooling section at a third temperature and receive a fourth coolant into the fourth cooling section to maintain temperature of the ODH catalyst in the fourth cooling section at a fourth temperature, wherein the third temperature is lower than the fourth temperature.

28. The system of claim 27, wherein the first temperature and the third temperature each are in a range of 300° C. to 450° C., wherein the second temperature and the fourth temperature each are in a range of 350° C. to 500° C., and wherein the steam in the feed acts as a diluent to place the feed outside of flammability limits.

29. The system of claim 27, comprising:
a first-coolant supply system comprising a pump to provide the first coolant to the first cooling section and maintain temperature increase of the first coolant through the first cooling section to below a first threshold;
a second-coolant supply system comprising a pump to provide the second coolant to the second cooling section and maintain temperature increase of the second coolant through the second cooling section to below a second threshold;
a third-coolant supply system comprising a pump to provide the third coolant to the first cooling section and maintain temperature increase of the third coolant through the third cooling section to below a third threshold; and
a fourth-coolant supply system comprising a pump to provide the fourth coolant to the fourth cooling section and maintain temperature increase of the fourth coolant through the fourth cooling section to below a second threshold.

30. The system of claim 29, wherein the first threshold and the second threshold are in a range of 2° C. to 8° C., and wherein the first coolant and the second coolant each comprise molten salt.

31. The system of claim 27, comprising an oxygen supply conduit to inject oxygen into the first effluent flowing through the first-effluent discharge conduit.

32. The system of claim 31, comprising an injection-water conduit to inject liquid water into the first effluent flowing through the first-effluent discharge conduit to cool the first effluent.

33. The system of claim 31, comprising a heat exchanger disposed along the first-effluent discharge conduit to cool the first effluent with water, thereby heating the water for generating steam from the water.

34. The system of claim 31, wherein the first ODH reactor comprises a fifth cooling section operationally downstream of second cooling section to cool the first reaction mixture flowing through the first tube side in the fifth cooling section, wherein the fifth cooling section and the second cooling section are separated by a second flow barrier on the first shell side.

35. The system of claim 34, wherein the first tube side in the fifth cooling section does not comprise catalyst.

36. The system of claim 27, comprising a heat exchanger to heat water with the first coolant discharged from the first cooling section for generating steam from the water.

37. The system of claim 36, comprising a superheater heat exchanger to heat the steam with the first coolant from the first shell side or with the second coolant from the first shell side, or both, to superheat the steam.

38. The system of claim 27, comprising a heat exchanger to heat water with the second coolant discharged from the second cooling section for generating steam from the water, wherein the first ODH reactor is configured to generate acetic acid from ethane in the first reaction mixture flowing through the first tube side.

39. The system of claim 27, comprising a heat exchanger to heat water with the third coolant discharged from the third cooling section for generating steam from the water wherein the second ODH reactor is configured to generate acetic acid from ethane in the second reaction mixture flowing through the second tube side.

40. The system of claim 27, comprising a heat exchanger to heat water with the fourth coolant discharged from the fourth cooling section for generating steam from the water.

41. The system of claim 27, comprising a heat exchanger disposed along the second-effluent discharge to heat water with the second effluent for generating steam from the water.
